SEQUENCE LISTING (2 sequences)

SEQ ID NO: 1 - protein, 16 aa, Artificial Sequence
  Synthetic Construct
GCRDVPMSMRGGDRCG

SEQ ID NO: 2 - protein, 7 aa, Artificial Sequence
  Synthetic Construct
GRGDSPC

(19) United States
(12) Patent Application Publication
(10) Pub. No.: US 2024/0210384 A1
(43) Pub. Date: Jun. 27, 2024

(54) CELL POTENCY ASSAYS, PLATFORMS, AND METHODS OF USE

(71) Applicants: GEORGIA TECH RESEARCH CORPORATION, Atlanta, GA (US); EMORY UNIVERSITY, Atlanta, GA (US)

(72) Inventors: Rebecca S. SCHNEIDER, Atlanta, GA (US); Andres J. GARCIA, Atlanta, GA (US); Wilbur A. LAM, Atlanta, GA (US); Evelyn Kendall WILLIAMS, Atlanta, GA (US); Karen E. MARTIN, Atlanta, GA (US)

(21) Appl. No.: 18/558,026

(22) PCT Filed: Apr. 29, 2022
(86) PCT No.: PCT/US2022/027056

§ 371 Date: Oct. 30, 2023

Related U.S. Application Data
(60) Provisional application No. 63/182,075, filed on Apr. 30, 2021.

(51) Int. Cl. G01N 33/50

(52) U.S. Cl. CPC G01N 33/5091; G01N 2333/57; G01N 2333/96494

(57) ABSTRACT

A high-throughput, scalable, low-cost, on-chip microfluidic potency assay for nondifferentiated cells such as human mesenchymal stromal cells (hMSCs) with improved functional predictive power and recapitulation of in vivo secretory responses compared to traditional approaches. By comparison of hMSC secretory responses to functional hMSC-medicated immune cell suppression, on-chip microfluidic potency markers are identified with improved functional predictive power compared to traditional planar methods.

### CROSS-REFERENCE TO RELATED APPLICATIONS

This application claims benefit of U.S. Provisional Application No. 63/182,075, filed Apr. 30, 2021, incorporated herein by reference in its entirety.

### GOVERNMENT SUPPORT CLAUSE

This invention was made with government support under Grant No. EEC-1648035 from the National Science Foundation and Grant Number AR062368 from the National Institutes of Health. The government has certain rights in the invention.

### BACKGROUND

Bone-marrow derived human mesenchymal stromal cells (hMSCs), also termed mesenchymal stem cells, represent a promising cell therapy candidate for their anti-inflammatory and immunomodulatory properties. Despite almost three decades of clinical development for diverse inflammatory and immune disease indications (Pittenger 2019) no hMSC product has yet to reach the US market. Whereas early phase hMSC clinical research had promising results, setbacks were faced at advanced clinical phases as endpoints failed to be met (Levy 2020; Martin 2019; Wechsler 2021). For example, the multicenter Prochymal® phase III clinical trial (Osiris Therapeutics Inc., now Mesoblast Inc., NCT00366145) of hMSC treatment for steroid-resistant graft-versus-host disease had disappointing outcomes despite promising early phase results (Galipeau 2013). More recently, the US Food and Drug Administration (US FDA) rejected a biological license application (BLA 125706, August 2020) by Mesoblast for a similar hMSC product due to the inability to show defined potency attributes representative of in vivo or clinical performance and resulting lack of assurance in consistent manufacturing processes (FDA 2020).

These fast-to-phase III clinical advances bypass robust assay development for therapeutic characterization (Bravery 2013), and the information gleamed from these assays is often retrospectively blamed for inconsistent manufacturing processes and later-stage clinical failures. FDA applications for new drug investigations require defined potency metrics, i.e. quantitative measures of biologic function, as a measure of quality and consistency for product scale-up and release (FDA 2011). Where direct measurement of biologic function is not feasible, surrogate markers of potency are recommended (Bravery 2013). The FDA has acknowledged challenges in pursing this task for therapeutics with unclear and/or pleiotropic mechanisms, as is the case for hMSCs. To this point, hMSC potency assay development has had poor demonstrated success with limited assay robustness, high product variability, and inadequacy to support clinical needs.

Whereas hMSC mechanism(s) of action is multifaceted, the leading hypothesis implicates hMSC immunomodulation of host immune cells (notably T cells) by hMSC secreted factors. Thus, hMSC-mediated T cell suppression in hMSC:peripheral blood mononuclear cell (PBMC) co-cultures is a well-accepted functional metric for hMSC immunomodulatory potential, as well as endorsed by the International Society for Cellular Therapy (ISCT) (Robb 2019; Galipeau 2016). However, this metric has severe limitations as a scalable and reproducible potency assay, due to significant variability of PBMC donors and limitations on scalability of the assay (Chinnadurai 2018). To overcome these challenges, alternative hMSC biomarkers have been explored as surrogate metrics of potency, such as indoleamine 2,3-dioxygenase (IDO) (Levy 2020; Robb 2019) or programmed death-ligand 1 (PD-L1) (Levy 2020; Chinnadurai 2014) in response to defined inflammatory stimuli, such as interferon-gamma (IFN-γ) (Chinnadurai 2018). These surrogate potency markers have been proposed based on marker correlation to T cell suppression levels and further validated by pathway inhibitors to restore T cell proliferation.

Although hMSCs in vivo serve as vital proteomic and structural mediators of the soft bone marrow niche (Nguyen 2018; Nakahara 2019), in these in vitro potency assays hMSCs are cultured on stiff planar substrates. These stiff substrates are well known to have profound influence on critical cellular processes such as proliferation, migration, differentiation, and have specifically been shown to bias hMSCs towards osteogenic fate (Trappmann 2012; Swift 2013). hMSC potency assays developed using stiff culture surfaces have so far lacked success in creating robust and translatable metrics. Whereas several surrogate potency markers have been proposed, none have yet demonstrated relevance to hMSC secretory performance following in vivo delivery.

On-chip microfluidic technologies (or tissue-chips) have gained interest as a tool in drug development and patient diagnostics for their ability to introduce physiological stimuli to in vitro systems (Low 2017). These micro-scaled systems often include fluid flow and 3D cellular spatial arrangements, enabling well-controlled and precise mechanical stimuli for improved recapitulation of in vivo environments. Specifically, the improved physiological relevance of on-chip microfluidic systems can help to overcome the poor pre-clinical translation observed for current in vivo models of hMSC (Galipeau 2018). While on-chip microfluidic technologies have shown significant potential in translational research, the high manufacturing costs and long lead time to produce these complex tissue-mimetic systems have limited their adoption.

What is needed in the art is a high-throughput, scalable, and low cost 3D microfluidic hMSC potency assay and platform. These 3D microfluidic system can provide greater functional predictive power and improved secretory recapitulation of cells delivered in vivo compared to traditional 2D assays.

### SUMMARY

Disclosed herein is a microfluidic potency assay comprising living cells, wherein the cells are encapsulated in a synthetic hydrogel, and further wherein the hydrogel is incorporated into a system which is perfused with liquid media and/or disease-relevant chemical stimuli.

Also disclosed is a method of assessing health or in vivo secretion of a cell culture, the method comprising: a microfluidic potency assay comprising living cells, wherein the cells are encapsulated in a poly(ethylene glycol) (PEG) hydrogel, and further wherein the hydrogel is incorporated into a sealed system which is perfused with liquid media and/or disease-relevant chemical stimuli; obtaining one or more liquid samples from the cell culture; and detecting one or more markers in the sample or samples obtained from the cell culture; and analyzing said markers to assess the viability, health, or in vivo secretion of the cell culture.

### BRIEF DESCRIPTION OF THE DRAWINGS

FIG. 1A-E shows that healthy hMSC donors demonstrate various T cell suppressive ability. (a) Nine donors from three manufactures were expanded as per manufacturer protocol. (b) Population doubling level (PDL) reported from time of receipt of cell line. (c) Microscope phase contrast images of each hMSC donor line. (d) Schematic of T cell suppression protocol. (e) CD3+ T cell suppression for various hMSC dose normalized to activated PBMC-only control (dotted line). One PBMC donor shown across three repeated experiments. Two-way ANOVA comparing cell ratio (or dose) mean to activated PBMC-only control. ****P<0.0001; n≥38 across 9 hMSC donors. All data represented as means±SEM.

FIG. 2A-F shows evaluation of IDO as a potency metric in 2D IFN-γ system. (a) Schematic of 2D experimental design. (b) Image flow cytometry of brightfield, intracellular IDO, and surface bound PD-L1. (c) IDO expression (left) and IDO activity (right) upregulated from 2D Ctrl. (d) Linear regression analysis of suppression index to IDO expression (left) and IDO activity (right). (e) Summary linear regression R2 of suppression index to IDO (left) and PD-L1 (right) metrics. (f) Linear regression of 2D IFN-γ IDO expression and activity. Mean fluorescent intensity (MFI) normalized to 2D ctrl (MFI MFI⁻¹) for comparison across repeated experiments. Bar graphs: Two-tailed unpaired t test with Welch's correction used. ***P<0.0005, ****P<0.0001, dotted red line as 2D ctrl mean. Linear regression: significant for P<0.05. Solid black lines are best fit lines, dotted black lines as 95% confidence bands. Summary linear regression: Corresponding *P<0.0332. **P<0.0021; n=44 across 9 donors. All data represented as means±SEM.

FIG. 3A-C shows a microfluidic system informed from secretion of hMSCs delivered in vivo. (a) Schematic of microfluidic synthesis including hydrogel crosslinking, cell encapsulation, and pressure-driven media perfusion. (b) Image of microfluidic chip indicating inlet and outlet channels, PDMS base, and hydrogel location. (c) Iterative approach for development of analyte panel and design parameters informed from subcutaneous delivered hMSC-laden hydrogel mouse model.

FIG. 4A-F shows the evaluation of IDO and PD-L1 as a potency metric in microfluidic IFN-γ system. (a) Schematic of microfluidic IFN-γ experimental design. (b) Heat map and two-way hierarchal clustering of analyte secretion across 2D IFN-γ and microfluidic IFN-γ culture systems. Clustering by Ward Method (pg pg-1). (c) IDO expression (left) and IDO activity (right) compared to 2D Ctrl. (d) Linear regression analysis of suppression index to IDO expression (left) and IDO activity (right). (e) Summary linear regression R2 of suppression index and IDO (left) and PD-L1 (right) metrics. (f) Linear regression of microfluidic IFN-γ IDO expression and activity. Mean fluorescent intensity (MFI) normalized to 2D ctrl (MFI MFI-1) for comparison across repeated experiments. Bar graphs: Two-tailed unpaired t test with Welch's correction used. ****P<0.0001, dotted red line as 2D Ctrl mean. Linear regression: significant for P<0.05. Summary linear regression: Corresponding *P<0.0332; n=38 across 9 donors. All data represented as means±SEM.

FIG. 5A-E shows a comparison of analyte secretion in 2D IFN-γ and microfluidic IFN-γ systems. (a) Summary linear regression R2 for suppression index values, of various T cell subsets and hMSC dose, and secretion levels of 20 analytes across both 2D IFN-γ and microfluidic IFN-γ culture systems. (b) Summary linear regression R2 for suppression index and MMP-13 secretion. (c) Top five R2 values for CD3+ 1:2 suppression index for all metrics both 2D IFN-γ and microfluidic IFN-γ systems. (d) Contrasting correlative trends of suppression index (CD4+ 1:4) and TNF R1 secretion in microfluidic IFN-γ or 2D IFN-γ systems. (e) Summary linear regression R2 (left) and corresponding best fit slopes (right) showing variable correlative confidence but consistent contradicting trends between culture systems. Analyte secretion (pg mL-1) normalized to 2D Ctrl (pg pg-1) for comparison across repeated experiments. Summary graph: *P<0.0332, **P<0.0021, ****P<0.0001 for corresponding P values; (+) positive slope, (−) negative slope. Linear regression analysis: black solid lines as best fit lines, dotted lines as 95% confidence bands; correlation significant for P<0.05. Microfluidic IFN-γ n=38, 2D IFN-γ n=44, across 9 donors. All data represented as means±SEM.

FIG. 6A-E shows in vivo signaling pathways recapitulated in microfluidic IFN-γ but not 2D IFN-γ systems. (a, b) Positive correlation in vivo between local inflammatory signals IFN-γ and (a) TNF-α and (b) TNF R1. (c-e) Inflammatory signaling pathways recapitulated in microfluidic IFN-γ system, but not 2D IFN-γ system, compared to in vivo-delivered hMSCs for TNF-α and (c) TNF R1 (d) IL-17E and (e) MMP-13. Solid black lines as best fit lines, dotted lines as 95% confidence bands; correlation significant for P<0.05. Microfluidic IFN-γ n=38 and 2D IFN-γ n=44 across 9 donors; in vivo n≥30 across 3 donors.

FIG. 7A-B shows hMSC-mediated T cell suppression across CD4⁺ and CD8⁺ T cell subsets. Suppression index of T cell populations CD3⁺CD4⁺ (a) and CD3⁺CD8⁺ (b), normalized to activated PBMC-only control. Two-way ANOVA statistical analysis across hMSC:T cell ratio mean differences against activated PBMC-control (dotted line). *P<0.0332; ****P<0.0001. Data represents 3 independent experiments with single PBMC donor (Donor 1); n=2-9 biological replicates. All data represented as means±SEM.

FIG. 8A-B shows two PBMC donors comparing hMSC-mediated T cell suppression. hMSC donors had consistent relative T cell suppression levels across two PBMC donors, PBMC Donor 1 (a) and PBMC Donor 2 (b). Different absolute suppressions were observed, demonstrating limitations of co-culture assay robustness and reproducibility. Two-way ANOVA performed between hMSC donor suppression for each hMSC:T cell ratio. **P<0.0021; ns, no significance; n=3-9 biological replicates. All data represented as means±SEM.

FIG. 9 shows IDO activity of hMSC-T cell co-culture supernatant. IDO activity measured as byproduct L-kynurenine (ng mL⁻¹) measured in the cell supernatant following three days of hMSC and T cell co-culture with anti-CD3⁺/anti-CD28⁺ activation beads. Two-way ANOVA statistical test performed across hMSC:T cell ratio mean differences. ***P<0.0002; ****P<0.0001; ns, no significance; n=3 per donor. All data represented as means±SEM.

FIG. 10A-C shows intracellular IDO and surface bound PD-L1 imaging flow cytometry. Images of IDO and PD-L1 imaging flow cytometry of TAMU 8011 hMSC donor with and without IFN-γ stimulation (a). Results show intracellular IDO and surface PD-L1 localization. Three representative images shown of 1000 events recorded. Fluorescent minus one (FMO) controls shown (b-c).

FIG. 11A-B shows IDO expression measured as % IDO positive. IDO expression (% IDO positive) upregulated from 2D Ctrl (a). Linear regression analysis of suppression index to IDO expression (% IDO positive) (b). Bar graphs: Two-tailed unpaired t test with Welch's correction used. ***P<0.0005, ****P<0.0001, dotted red line as 2D ctrl mean. Linear regression: significant for P<0.05. N=44 across 9 donors. All data represented as means±SEM.

FIG. 12 shows linear regression of suppression index and IDO activity in 2D IFN-γ normalized to cell count. Linear regression analysis of IDO Activity normalized to cell count (ng cell⁻¹) against suppression index for 2D IFN-γ culture system. Cell count calculated using flow cytometry counting beads for one unique experiment. 2D IFN-γ n=26 across 9 donors. Dotted lines represent 95% confidence bands, significant for P<0.05. All data represented as means±SEM.

FIG. 13A-E shows hMSC PD-L1 potency and regulation analysis in 2D INF-γ culture. Upregulation of surface bound and soluble PD-L1 compared to 2D Ctrl (a-b). Negative correlation between surface bound and soluble forms of PD-L1 in 2D IFN-γ culture (c). Regression analysis of suppression index and PD-L1 metrics in 2D IFN-γ culture (d-e). Data normalized to 2D Ctrl (MFI MFI⁻¹, pg pg⁻¹). Bar graphs: Two-tailed unpaired t test with Welch's correction used. ****P<0.0001. Linear regression analysis: black solid line, best-fit line; black dotted lines, 95% confidence bands. Red dashed lines, baseline 2D Ctrl. Significant for P<0.05. N=44 across 9 donors. All data represented as means±SEM.

FIG. 14 shows the fluorescent imaging of perfusion through hydrogel in microfluidics system. Media containing FITC-dextran (20 kDa) was perfused to visualize anticipated IFN-γ (17 kDa) distribution and bioavailability. FITC-dextran shown throughout the hydrogel. Image represents maximum projection of z-stacked image.

FIG. 15A-B shows hMSC secretion comparing of 2D, 3D static, and microfluidic systems. Hierarchal clustering with representative heat map (a) and principal component analysis show secretory responses (pg) of microfluidic IFN-γ cluster separate from 3D static IFN-γ, 2D IFN-γ, and 2D Ctrl culture systems (b). Data collected following 4 days of culture with n=2-3 per group. Graphs generated using SAS JMP Pro 15 software.

FIG. 16A-B shows the development of subcutaneous model to characterize in vivo hMSC-delivered secretion (a). Initial in vivo testing of hMSC-laden hydrogel (Hydrogel+hMSC), hMSC in solution (hMSC soln), cell-free hydrogel (Hydrogel), and saline. First steps in developing in vivo model and characterizing in vivo hMSC-delivered secretion profile. Total protein measured with BCA protein assay (b). Single experiment shown, n=3. One-way ANOVA statistical test performed on log-transformed data. **P<0.0021, ***P<0.0002, ****P<0.0001; ns, no significance. All data represented as means±SEM.

FIG. 17 shows hMSC donor characterization of hMSC-delivered in vivo secretion. Subcutaneous in vivo model tested across three hMSC donors (TAMU 8011, TAMU 8013, TAMU 7083) and cell-free hydrogel (Gel) control. One to three experiments shown (n=3-7 per experiment). Detection limits and measurement sensitivities varied by Luminex kit; analytes below detection were set at the lowest limit. Concentration reproducibility varied between kits and seemed dependent on analyte measured. (Bottom, right) Total protein measured with BCA protein assay. One-way ANOVA statistical test performed, data log transformed as indicated. *P<0.0332, **P<0.0021, ***P<0.0002, ****P<0.0001; ns, no significance. All data represented as means±SEM.

FIG. 18A-B shows hMSC-delivered in vivo secretion of local IFN-γ. In vivo IFN-γ secretion tested across single-component controls (hMSC soln, hydrogel) (a). One experiment, n=3. In vivo IFN-γ secretion tested across three donors in vivo (b). Three experiments (n=12-17). IFN-γ presence of hMSC-delivered in vivo supports exogenous IFN-γ stimulation in in vitro system. Detection limits and measurement sensitivities varied by Luminex kit; analytes below detection were set at the lowest limit. One-way ANOVA statistical test performed of log-transformed data. **P<0.0021, ***P<0.0001; ns, no significance. All data represented as means±SEM.

FIG. 19 shows the in vivo analysis of mouse analyte levels for investigation of host response to hMSC-laden hydrogel injection. No significant change in local host mouse cytokine levels following hydrogel injection with or without hMSC. Suggests hMSC introduction does not elicit strong host immune response and that human analytes measured are products of hMSC secretion. One-way ANOVA statistical test performed. ns, no significance; ND, no detection. All data represented as means±SEM.

FIG. 20 shows one iteration of in vivo and in vitro discriminant analysis as per analyte panel iterative design processes. Discriminant analysis for characterization of secretory similarities between in vivo (subcutaneous) and in vitro (microfluidic IFN-γ) models. Discriminant analysis method: linear, common covariance. Discriminant classification by donor. Ctrl: cell-free gel (in vivo), microfluidic without IFN-γ stimulation (in vitro). Input data as net MFI. Inner ellipse represents 95% confidence, outer ellipse represents 50% prediction. N≥3. Graph generated using SAS JMP Pro 15 software.

FIG. 21A-C shows the discriminant analysis of 2D IFN-γ culture differs from microfluidic IFN-γ and in vivo analysis. Discriminant analysis of hMSC secretory response of in vivo (a), microfluidic IFN-γ (b), and 2D IFN-γ (c) models show greater similarity across in vivo and microfluidic IFN-γ compared to 2D IFN-γ. Discriminant analysis method: linear, common covariance. Discriminant classification is by donor. Ctrl groups; in vivo: cell-free gel, microfluidic IFN-γ: microfluidic without IFN-γ stimulation, 2D IFN-γ: 2D Ctrl. Input data as net MFI; n≥6. Inner ellipse represents 95% confidence; outer ellipse represents 50% prediction. Graph generated using SAS JMP Pro 15 software.

FIG. 22 shows cell viability measured across different systems. Viability measured in tandem with flow cytometry and secretory results shown. Measured using fixable live/dead stain by flow cytometry. Two-way ANOVA statistical test performed across system means; microfluidic IFN-γ n=38, 2D IFN-γ n=44, 2D Ctrl n=45. ****P<0.0001. All data represented as means±SEM.

FIG. 23 shows cell viability partially recovered in 3D static culture system. Viability measured using fixable live/dead stain by flow cytometry. Single donor TAMU 8011 used. Two-way ANOVA statistical test performed across system means. N=4 per group. ****P<0.0001. All data represented as means±SEM.

FIG. 24A-B shows the secretory response of hMSC donors clusters by culture system. Hierarchal clustering with representative heat map shows samples cluster by culture system (2D IFN-γ, microfluidic IFN-γ) with sub-clustering by manufacturer (TAMU, RB, LONA) (a). Clustering by Ward Method, standardized by analyte. Principal component analysis supports clear distinction of secretory response between culture systems (b). Samples normalized 2D Ctrl (pg pg⁻¹) for each analyte and unique experiment. Microfluidic IFN-γ represented as filled symbols (n=38), 2D IFN-γ represented as bordered symbols (n=44). Graphs generated using SAS JMP Pro 15 software.

FIG. 25A-D shows the comparison of IDO levels between culture systems. IDO expression (a), IDO activity (b), and final cell count (c) compared between microfluidic IFN-γ, 2D IFN-γ, and 2D Ctrl culture systems. Differences in cell proliferation between culture systems impacts IDO activity. Other inherent differences across culture systems including total media fed, media collected, and total IFN-γ delivered (d). Two-way ANOVA statistical test performed across culture system mean; microfluidic IFN-γ n=38, 2D IFN-γ n=44, 2D Ctrl n=45. *P<0.0332, **P<0.0021, ****P<0.0001; ns, no significance. All data represented as means±SEM.

FIG. 26 shows experimental consistency for intracellular IDO maintained for microfluidic IFN-γ but not 2D IFN-γ culture. Shows IDO expression (normalized 2D Ctrl, MFI MFI⁻¹) from samples collected from two repeated experiments. Samples show good consistency for microfluidic IFN-γ system and high variance for 2D IFN-γ culture. N=5-6 per donor per experiment. All data represented as means±SEM.

FIG. 27 shows donor-matched correlation between IDO in microfluidic IFN-γ and 2D IFN-γ culture systems. Linear regression analysis comparing donor-specific microfluidic IFN-γ and 2D IFN-γ IDO expression (left) and IDO activity (right) metrics. Correlative for P<0.05. Microfluidic IFN-γ n=38, 2D IFN-γ n=44 across 9 donors. All data represented as means±SEM.

FIG. 28A-E shows hMSC PD-L1 potency and regulation analysis in microfluidic IFN-γ culture. Upregulation of surface bound (a) and soluble (b) PD-L1 compared to 2D Ctrl. Negative correlation between surface bound and soluble forms of PD-L1 in microfluidic IFN-γ culture (c). Regression analysis of suppression index and PD-L1 metrics in microfluidic IFN-γ culture (d-e). Data normalized to 2D Ctrl (MFI MFI⁻¹, pg pg⁻¹). Bar graphs: Two-tailed unpaired t test with Welch's correction used.***P<0.002 ****P<0.0001. Linear regression analysis: black solid line, best-fit line; black dotted lines, 95% confidence bands. Red dashed lines, baseline 2D Ctrl. Significant for P<0.05. N=38 across 9 donors. All data represented as means±SEM.

FIG. 29 shows the evaluation of analyte potency utility in 2D IFN-γ and microfluidic IFN-γ systems. Linear regression analysis performed on secretion levels of 20 analytes of 2D IFN-γ and microfluidic IFN-γ culture systems. Linear regression performed for donor-matched T cell suppression index (CD3⁺ 1:2) versus analyte secretion levels.

Analyte levels measured with multiplex Luminex assay and normalized to 2D Ctrl (pg pg⁻¹) for comparison across independent experiments. Outlined symbols are 2D IFN-γ cultures and filled symbols are microfluidic IFN-γ cultures. Linear regression analysis: black is microfluidic IFN-γ system and red is 2D IFN-γ culture best fit lines. Surrounding dotted lines are 95% confidence bands. Correlation significant for P<0.05. Horizontal dotted line is 2D Ctrl. Microfluidic IFN-γ n=38, 2D IFN-γ n=44 across 9 donors. All data represented as means±SEM.

FIG. 30 shows MMP-13 had correlative consistency across suppression index T cell subset, hMSC dose, and culture systems. MMP-13 had consistent correlation and positive best fit slope to suppression index for all variations, including: T cell subsets (CD3⁺, CD3⁺CD4⁺, CD3⁺CD8⁺), hMSC dose (hMSC:T cell ratio 1:2, 1:4, 1:8), and culture system (microfluidic IFN-γ, 2D IFN-γ). This consistency was unique to secreted MMP-13 analyte. Secretion data normalized to 2D Ctrl (pg pg⁻¹) for comparison across independent experiments.

FIG. 31 shows the experimental consistency for MMP-13 in microfluidic IFN-γ system. Low donor-specific variance observed for MMP-13 levels in microfluidic IFN-γ culture across independent experiments. N=4-8 replicates per donor per experiment. All data represented as means±SEM.

FIG. 32A-B shows in vivo MMP-13 secretion consistent with in vitro suppression index trends. In vivo MMP-13 secretion (a) and in vitro functional T cell suppression (b) show consistent trends where in vivo donor TAMU 8013 demonstrated no increase in MMP-13 secretion compared to cell-free gel and in vitro donor TAMU 8013 had significant T cell suppression compared to activated PBMC-only control. In vivo data is consistent with in vitro MMP-13 trends where lower MMP-13 levels corresponds to more potent product. In vivo, one-way ANOVA statistical test performed on log-transformed data. In vitro, one-way ANOVA against PBMC-activated control (dotted line). *P<0.0332; ns, no significance. All data represented as means±SEM.

FIG. 33 shows donor-matched correlation of 2D IFN-γ and microfluidic IFN-γ culture secretion. Of secreted analytes measured, 5 of 20 (25%) had positive correlative trends, 1 of 20 (5%) had negative correlative trends, and 14 of 20 (70%) had no correlation. Data reported as (pg pg⁻¹) normalized to 2D Ctrl. Linear regression: significant for P<0.05. Solid black lines are best fit lines, dotted black lines are 95% confidence bands. N=38 across 9 donors. All data represented as means±SEM.

FIG. 34 shows secretion of 20 analytes across microfluidic IFN-γ, 2D IFN-γ, and 2D Ctrl cultures. Non-normalized secretion levels grouped by linear regression correlation as indicated. No obvious patterns were recognized. *P<0.0332, **P<0.0021, ***P<0.0002, ****P<0.0001; ns, no significance. Dotted lines represent background (media) and/or assay maximum detection limit (max). All data represented as means±SEM.

FIG. 35A-C shows microfluidic analyte potency trends uniquely maintained with perfusion stimuli. Analyte trends with potency were not maintained for static 3D IFN-γ system when comparing more potent RB 177 donor and less potency TAMU 8011 donor (a-c). Two-way ANOVA statistical test performed between donor means for each system; n=2-3. *P<0.0332, **P<0.0021, ***P<0.0002, ****P<0.0001; ns, no significance. All data represented as means±SEM.

FIG. 36 shows R² summary for non-normalized analyte analysis. Linear regression summary for suppression index and non-normalized analyte secretion (pg mL⁻¹) had overall reduced correlation (R²) and confidence (P value) compared to normalized analysis. Summary plot shows 20 secreted analytes across both 2D IFN-γ and microfluidic IFN-γ systems for suppression index of various T cell subsets (CD3⁺, CD3⁺CD4⁺, CD3⁺CD8⁺) and hMSC dose (hMSC:T cell 1:2, 1:4, 1:8). Dotted lines show approximate corresponding P values, where *P<0.0332, **P<0.0021, ***P<0.0002.

FIG. 37A-D shows in vitro correlative trends of MMP-13 and PD-L1. MMP-13 mediated PD-L1 cleavage uniquely supported by correlative trends in microfluidic IFN-γ cultures. Dotted lines represent 95% confidence bands. Significant for P<0.05. Microfluidic IFN-γ n=38 (a,c), 2D IFN-γ n=44 (b,d).

FIG. 38 shows donor-matched TNF R1 microfluidic IFN-γ and 2D IFN-γ correlation. Secreted TNF R1 had negative correlation between 2D IFN-γ and microfluidic IFN-γ cultures. Linear regression analysis: solid black lines as best fit lines, dotted lines as 95% confidence bands; correlation significant for P<0.05. Microfluidic IFN-γ n=38, 2D IFN-γ n=44. All data represented as means±SEM.

FIG. 39A-F shows the in vivo secretion correlative trends to IFN-γ signaling. Local hMSC secretion correlative trends for potent inflammatory signaling que IFN-γ following subcutaneous in vivo hMSC delivery (a-f). Linear regression: significant for P<0.05. Solid black likes are best fit lines, dotted black lines are 95% confidence bands; n=43.

FIG. 40A-B shows lack of detection in vivo IDO activity and soluble PD-L1 levels. IDO activity (a) and soluble PD-L1 (b) were at/below the detection limit for in vivo samples. For IDO activity, dotted line represents detection limit as determined by standard curve. Donor samples were not different from cell-free gel control. For soluble PD-L1, all samples were at/below detection and set at the lowest limit. One-way ANOVA statistical test performed. Ns, no significance. N=5 per group. All data represented as means±SEM.

FIG. 41A-D shows the cluster of correlations of secreted analytes across systems. 19 analytes analyzed as by correlative clusters (PD-L1 excluded, below detection for in vivo samples). In vivo samples have high positive correlative trends, followed by microfluidic IFN-γ cultures. Cluster of correlations analyzed as a multivariate method by SAS JMP Pro 15 software (d). In vivo (pg mL⁻¹) n=25-43 across 3 donors (a), microfluidic IFN-γ (pg) n=38 (b) and 2D IFN-γ (pg) n=44 (c) across 9 donors.

FIG. 42A-C shows the gating scheme for T cell suppression assay. T cell suppression assay flow cytometry results shown for two hMSC doses (hMSC:T cell 1:2 (a) and 1:4 (b)), and control wells of PBMC-only (no hMSC) for both activated (+anti-CD3/anti-CD28 beads) and not activated (−anti-CD3/anti-CD28 beads) (c).

FIG. 43A-C shows flow cytometry proliferation graphs for T cell suppression assay. X-axis represents CFSE fluorescence from FITC channel where decreased fluorescence (Gate M1) indicates cell division or proliferation. Bottom row shows PBMC-only controls both activated (+anti-CD3/anti-CD28 beads) and not activated (−anti-CD3/anti-CD28 beads) (a). Suppression index is quantified from percent of cells proliferated (% M1) divided by percent proliferated of activated PBMC-only control (% M1 Activated PBMC-only) (b).

FIG. 44 shows the rheological testing of hydrogel component of microfluidic system. Hydrogel composition of 6% PEG-4MAL, 80:20 VPM:DTT crosslinking peptide, 1 mM RGD ligand. Recorded at constant strain rate 0.01 within linear region. Storage modulus (G′) and loss modulus (G″) measured by varying angular frequency (rad s⁻¹). N=3.

FIG. 45 shows the gating scheme for flow cytometry IDO and PD-L1 across three culture systems. Shows samples collected from one of two repeated experiments. FITC channel used for fixable live/dead stain. Raw flow cytometry MFI analysis collected from different experiments are not used in direct comparison. Fluorescent minus one (FMO) and isotype controls used for determining gating schemes of live/dead, IDO⁺, and PD-L1⁺ where appropriate.

### DETAILED DESCRIPTION OF THE INVENTION

### Definitions

As used in the specification and claims, the term “and/or” used in a phrase such as “A and/or B” herein is intended to include “A and B”, “A or B”, “A”, and “B”.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments. Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

As used herein, the term “hydrogel” refers to a physically or chemically cross-linked polymer network that is able to absorb large amounts of water. They can be classified into different categories depending on various parameters including the preparation method, the charge, and the mechanical and structural characteristics. Reference can be made to S. Van Vlierberghe et al., “Biopolymer-Based Hydrogels As Scaffolds for Tissue Engineering Applications: A Review,” Biomacromolecules, 2011, 12(5), pp. 1387-1408, which is incorporated herein by reference.

As used herein, the term “microfabrication” is a concept that includes fabrication on a nanometer or micrometer level, including microfabrication and nanofabrication. Methods for microfabrication are well known in the art. Reference to certain microfabrication techniques that may be applicable in the invention include, for example, U.S. Pat. Nos. 8,715,436, 8,609,013, 8,445,324, 8,236,480, 8,003,300, as well as Introduction to Microfabrication (2004) by S. Franssila. ISBN 0-470-85106-6, each of which are incorporated herein by reference.

The term “microfabricated structure” as used herein is a concept that includes one or more structures occupying a two- or three-dimensional space, including a structure fabricated on a nanometer or micrometer scale. The term “two-dimensional” means on a surface in either vertical or horizontal space.

As used herein, the term “pharmacokinetics” refers to the actions of the body on a drug. Pharmacokinetic processes include, but are not limited to, absorption, distribution, metabolism, and elimination of drugs.

As used herein, the term “pharmacodynamics” refers to the actions of a drug on the body. Because certain classes of drugs exhibit similar effects on the body, pharmacodynamic properties determine the group in which a drug or agent is classified.

As used here, the term “PDMS” refers to the polymer poly(dimethylsiloxane). Polydimethylsiloxane (PDMS) belongs to a group of polymeric organosilicon compounds that are commonly referred to as silicones. PDMS is the most widely used silicon-based organic polymer, and is particularly known for its unusual rheological (or flow) properties. PDMS is optically clear, and, in general, oxygen-permeable, inert, non-toxic, and non-flammable. It is also called dimethicone and is one of several types of silicone oil (polymerized siloxane).

A “test agent” is any substance that is evaluated for its ability to diagnose, cure, mitigate, treat, or prevent disease in a subject, or is intended to alter the structure or function of the body of a subject. A test agent in an embodiment can be a “drug” as that term is defined under the Food Drug and Cosmetic Act, Section 321(g)(1). Test agents include, but are not limited to, chemical compounds, biologic agents, proteins, peptides, antibodies, nucleic acids, lipids, polysaccharides, supplements, cells, cell fragments, diagnostic agents and immune modulators and may also be referred to as “pharmacologic agents.”

As used herein, the term “toxicity” is defined as any unwanted effect on human cells or tissue caused by a test agent, or test agent used in combination with other pharmaceuticals, including unwanted or overly exaggerated pharmacological effects. An analogous term used in this context is “adverse reaction.”

A “biofunctional hydrogel” is a hydrogel that contains bio-adhesive (or bioactive) molecules, and/or cell signaling molecules that interact with living cells to promote cell viability and a desired cellular phenotype. Biofunctional hydrogels may also be referred to as bioactive. These assays contain cell adhesive peptides that govern their interaction with cells. Examples of cell adhesion peptide sequences include, but is not limited to, RGD peptides, which are known to those of skill in the art. An example is SEQ ID NO: 2.

A “biocompatible hydrogel” is a polymer network that is not acutely toxic to living tissue and/or cells, and does not elicit an immunopathogenic response in healthy individuals. A biocompatible active mechanism is a process that is not toxic to particular cells or tissues, for example a temperature increase within the physiological temperature range of tissues, or that is applied briefly enough so as not to cause significant toxicity.

“Crosslinkable by cell-compatible reaction(s)” means that molecules are crosslinkable by reactions which are not significantly toxic to living tissue and/or cells. Such reactions may include (i) permanent covalent bond formation, chosen from the group consisting of a) enzymatically catalyzed reactions, preferably depending on activated transglutaminase such as factor Xllla; and b) not-enzymatically catalyzed and/or uncatalyzed reactions, preferably a Michael addition reaction; and/or ii) reversible covalent bond formation, chosen from the group consisting of Schiff base (imine) bonds, reversible hydrazone bonds, oxime bonds, disulfide bonds and bonds formed by reversible Diels-Alder reactions; and/or iii) non-covalent (i.e. physical) bond formation (e.g. on the basis of hydrophobic interactions, H-bonds, van-der-Waals, electrostatic interactions, host-guest interactions, biorecognition (domain/protein-ligand interactions); spontaneous or induced by temperature changes or changes in ionic strength of a buffer).

“Culturing cells” refers to the process of keeping cells in conditions appropriate for maintenance and/or growth, where conditions refers to, for example, the temperature, nutrient availability, atmospheric CO₂ content and cell density in which the cells are kept. Cells can be cultured in vivo or in vitro. The appropriate culturing conditions for maintaining, proliferating, expanding and differentiating different types of cells are often well-known and documented.

The term “composite material”, as used herein, is meant to refer to any material comprising two or more components. One of the components of the material can optionally comprise a matrix for carrying cells, such as a gel matrix or resin.

As used herein, the phrases “biologically active agent” and “biologically active factor” are used interchangeably and can refer to a compound or mixture of compounds that when added to a cell in culture induces the cell to enter differentiation (e.g., differentiate at least one step further along a pathway of differentiation).

As used herein, the term “effective amount” refers to an amount of a biologically active agent sufficient to produce a measurable response (e.g., a biologically relevant response in a cell exposed to the differentiation-inducing agent) in the cell. An effective amount of a differentiation-inducing agent can be an amount sufficient to cause a precursor cell to differentiate in in vitro culture into a cell of a tissue at predetermined site of treatment. It is understood that an “effective amount” can vary depending on various conditions including, but not limited to the stage of differentiation of the precursor cell, the origin of the precursor cell, and the culture conditions.

As used herein, the term “subject” refers to a vertebrate, preferably a mammal, more preferably a human being (male or female) at any age.

The phrase “in vivo” refers to within a living organism such as a plant or an animal, preferably in mammals, preferably, in human subjects.

As used herein, the phrase “ex vivo” refers to living cells which are derived from an organism and are growing (or cultured) outside of the living organism, preferably, outside the body of a vertebrate, a mammal, or human being.

The term “encapsulating cells” refers to encapsulating, entrapping, plating or placing cells within a hydrogel (or composition-of-matter). It will be appreciated that encapsulating the cells within a hydrogel can be performed following the formation of the hydrogel or prior to hydrogel formation, i.e., by mixing the cells with the aqueous solution containing the peptides and the polymer, as described herein for generating the hydrogel. The concentration of cells to be encapsulated in the hydrogels depends on the cell type and the hydrogel properties.

### Compositions and Methods

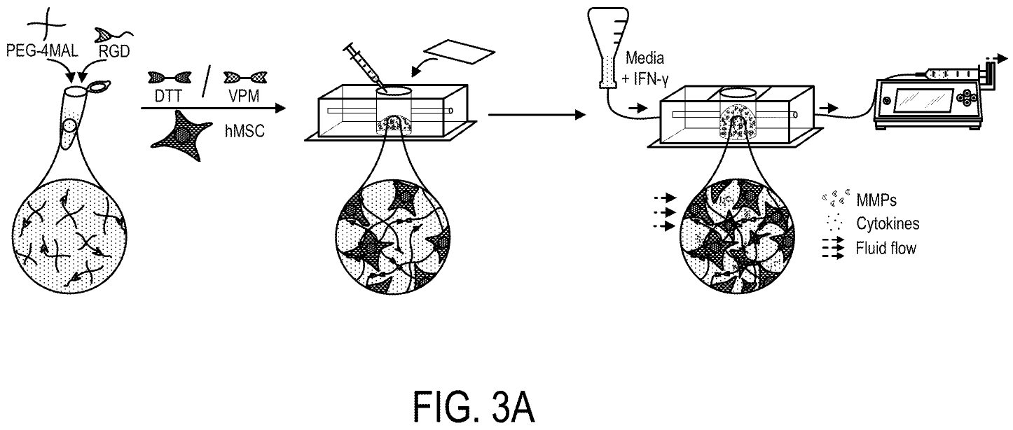

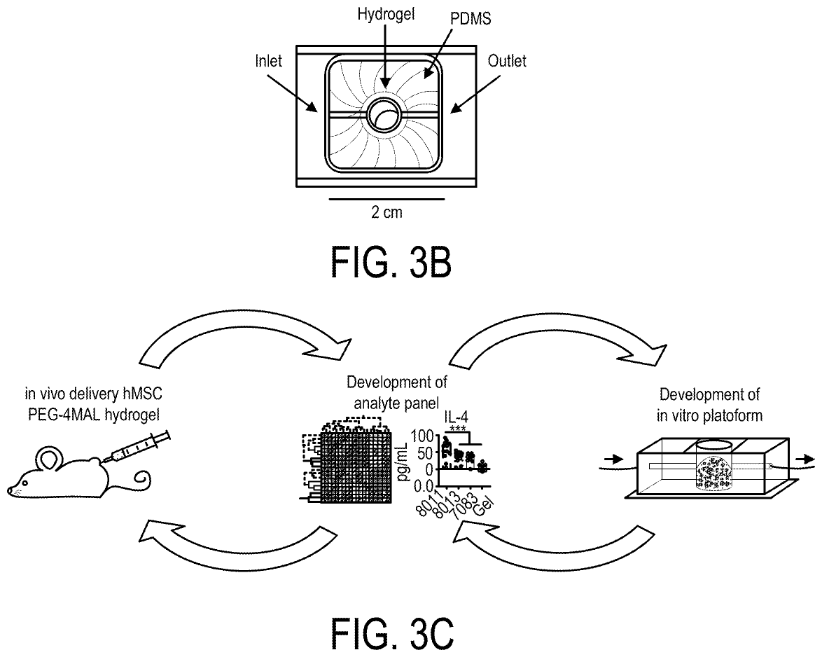

Disclosed herein is a microfluidic potency assay and device thereof comprising living cells, wherein the cells are encapsulated in a synthetic hydrogel, and further wherein the hydrogel is incorporated into a system perfused with liquid media and/or disease-relevant chemical stimuli. This assay can be used as a device, and can be scaled up and used in a high throughput manner, as discussed in more detail below. An example of the assay can be seen in FIGS. 3A-C.

The “sealed system” is sealed off so that it is not open to outside air. In one example, a pump is used to continually perfuse media through the assay device. This can be done, for example, with a peristaltic or fixed displacement/syringe pump.

The hydrogel can be any material known in the art which allows for the survival and/or growth of cells. This consists of, but is not limited to, three-dimensional hydrogels. The three-dimensional hydrogels of the invention can be specifically optimized for the culture and/or expansion living cells. The synthetic hydrogel can be selected from the group comprising poly(ethylene glycol), polyoxazoline, polyaliphatic polyurethanes, polyether polyurethanes, polyester polyurethanes, polyethylene copolymers, polyamides, polyvinyl alcohols, poly(ethylene oxide), polypropylene oxide, polypropylene glycol, polytetramethylene oxide, polyvinyl pyrrolidone, polyacrylamide, poly(hydroxy ethyl acrylate), poly(hydroxyethyl methacrylate), or mixtures or co-polymers thereof, for example. Alternatively, hydrogels sourced from natural materials (collagen, fibrin) can be used.

In an embodiment, the hydrogels used, which are obtained by cross-linking hydrogel precursor molecules, are preferably composed of hydrophilic polymers such as poly(ethylene glycol) (PEG)-based polymers, such as multiarm (i.e. branched) PEG-based polymers that are crosslinked by cell-compatible crosslinking reactions. Hydrogel precursors can be selected from a group comprising linear PEG molecules, or multiarm PEG hydrogel precursor molecules, such as those bearing 4-arms or 8-arms. For example, the hydrogel can be comprised of 4-arm maleimide-functionalized poly(ethylene-glycol) (PEG-4MAL).

In some embodiments, the hydrogel can be cross-linked with one or more protease-degradable or hydrolytic peptides that allow for cell morphological changes, including spreading. Examples include the peptide comprising VPM (GCRDVPMSMRGGDRCG, SEQ ID NO: 1). The hydrogel can also be cross-linked with a dithiolated molecule. For example, the reducing agent can comprise dithiothreitol (DTT). The hydrogel can also comprise an adhesive peptide to support cell adhesion, viability and function. An example of an adhesive peptide is RGD (GRGDSPC, SEQ ID NO: 2).

The cell type used in the assay can be used for therapeutic or research purposes. Examples of such cells include, but are not limited to, adult stem (totipotent, pluripotent, or multipotent) or stromal cells. This includes mesenchymal stromal cells (MSC), skeletal stem and progenitor cells, satellite cells, hematopoietic stem cells, stromal stem cells, neural precursor cells, liver precursor cells, skin precursor cells, epithelial stem cells, neural stem cells, and mesodermal precursor cells. These cells can be sourced from different tissues, including bone marrow, adipose tissue, placenta, etc.

Devices that can be utilized with the present invention include those described in U.S. Pat. Nos. 6,875,605 and 6,943,008. While the goal of these patents is different than the present invention, there are components of these bioreactors which can be used in the present system. In one embodiment, a perfusion device can have multiple perfusion chambers that can be controlled individually or in tandem. Transverse or parallel flow of a fluid can be provided to each chamber. The fluid is one that is capable of providing appropriate conditions for cell life and/or supporting and directing growth and/or differentiation of cells within the device, as discussed herein. For example, the fluid can be a fluid containing nutrients and other chemicals or factors, such as cytokines, to support the growth and/or differentiation of cells.

The media which perfuses the cells can comprise IFN-γ, TNF-α, TGF-β, IL-1, other cytokines, chemokines, mitogens, and growth factors as well cell-modulating chemical stimuli, including small molecules, cytoskeletal inhibitors, epigenetic modulators, metabolites, etc. The media can be liquid and contain biological factors supporting cell activities. Biological factors that may be in the conditioned medium include, but are not limited to, proteins (e.g., cytokines, chemokines, growth factors, enzymes), nucleic acids (e.g., miRNA), lipids (e.g., phospholipids), polysaccharides, small metabolites, cell fragments, and/or combinations thereof. Any combination(s) of these biological factors may be delivered bound within or on the surface of extracellular vesicles (e.g., exosomes) or separate from extracellular vesicles.

Medium perfusion can be done in a variety of ways, including, but not limited to, using fluidic tubing by gravity- or pressure-driven perfusion, reservoirs and microfabricated channels, fluid transport mediated by physical (e.g, motion—rocking, stirring, etc), electrical, and magnetic forces. This may comprise continuous, i.e. without ceasing, perfusion for a single time period, which may be prolonged and last preferably for a time period selected from any of 1 hour, 3 hours, 6 hours, 12 hours, 24 hours, 36 hours, 48 hours, 60 hours, 72 hours, 84 hours, 96 hours, 108 hours, 120 hours, 132 hours, 146 hours or a time period selected from within the range 1 to 146 hours. The perfusion flow rate, i.e. the rate of flow of culture media through the cell culture during perfusion, may be varied according to the growth characteristics of the cell type being cultured. Preferably, the perfusion flow rate is in the range 0.0005 to 5 mL/min and more preferably one of 0.001 mL/min, 0.005 mL/min, 0.01 mL/min, 0.02 mL/min, 0.04 mL/min, 0.05 mL/min, 0.5 mL/min, 1.0 mL/min or 2.0 mL/min.

The system can comprise a chip. For example, the chip can be fabricated from any durable material suitable for cell culture. Such material is known to those of skill in the art. For example, the material can be comprised of silicone, such as polydimethylsiloxane PDMS.

The assay disclosed herein can be used in a platform. For example, the assay can be designed to be high-throughput, so that large amounts of cells can be used in the assays. The platform can be scalable, so that 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more assays (devices) can be used together. This allows for an efficient, low-cost tool for assessing the viability, health, or in vivo secretion of a cell culture.

Also disclosed herein are methods of assessing viability, health, or in vivo secretion of a cell culture, the method comprising: providing a microfluidic potency assay comprising living cells, wherein the cells are encapsulated in a poly(ethylene glycol) (PEG) hydrogel, and further wherein the hydrogel is incorporated into a chip which is perfused with liquid media and/or disease-relevant chemical stimuli; obtaining one or more liquid samples from the cell culture; and detecting one or more markers in the sample or samples obtained from the cell culture; and analyzing said markers to assess the viability, health, or in vivo secretion of the cell culture.

The marker can be an analyte produced by the cell. This marker, or analyte, can be found, for example, in Table 3. 1, 2, 3, 4, 5, 6, 7, 8, 9, 10, 11, 12, 13, 14, 15, 16, 17, 18, 19, 20, or more of these markers can be used to assess the health or status of the cell culture. For example, the marker can be part of the autocrine or paracrine system. The analyte can be a secreted protein(s), cytokine, chemokine, enzyme, metabolite, or other cell product(s) or component(s). For example, during the step of analyzing, secretory response of the cell culture is analyzed, such as the cell's secretome. This analyzation can happen in a way that does not damage the cells. Methods of analyzing the secretome are known to those of skill in the art, and can be found, for example, in Pinho (2020). Metrics of secretion of the cell culture relevant to its physiological environment can be measured. Examples of analyzing markers in a cell culture can be found in U.S. Pat. No. 9,963,678, herein incorporated by reference in its entirety for its teaching concerning detecting markers of hMSC cell culture.

The cell performance can be compared to hydrogel in vivo, for example. The in vivo cell model can be used for assay creation, optimization, and/or validation. In one embodiment, the in vivo cell model introduces a cell-laden hydrogel polymerized in situ of the subcutaneous space of a subject. The subject can be an animal, such as a mammal. The cell performance can be detected by detecting at least one analyte such as MMP-13 either in vivo or in vitro.

The present invention relates to methods of using the assays, platforms, devices, and/or the systems of the invention in various applications, including, but not limited to, (a) the testing of the efficacy and safety (including toxicity) of experimental pharmacologic agents (including, but not limited to, small molecule drugs, biologics, nucleic acid-based agents, cell therapeutics), (b) the defining of pharmacokinetics and/or pharmacodynamics of pharmacologic agents (including, but not limited to, small molecule drugs, biologics, nucleic acid-based agents, cell therapeutics), (c) characterizing the properties and therapeutic effects of pharmacologic agents (including, but not limited to, small molecule drugs, biologics, nucleic acid-based agents, cell therapeutics) on a subject, (d) screening of new pharmacologic agents, (e) providing cells or tissues for use in regenerative medicine for treating damaged and/or diseased cells or tissues, and (f) personalized medicine.

### Example

Disclosed herein is a high-throughput, scalable, low-cost, on-chip microfluidic potency assay with improved functional predictive power and recapitulation of in vivo secretory responses compared to traditional approaches. By comparison of hMSC secretory responses to functional hMSC-medicated immune cell suppression, as demonstrated in the following example, the shortcomings of current surrogate potency markers and the identification of on-chip microfluidic potency markers with improved functional predictive power compared to traditional planar methods are shown. Furthermore, similar hMSC secretory performance is achieved in the on-chip microfluidic system compared to an in vivo model. These results underscore the shortcomings of current culture practices and present a novel system with improved functional predictive power and hMSC physiological responses.

Characterization of hMSC Donors for Potency Analysis

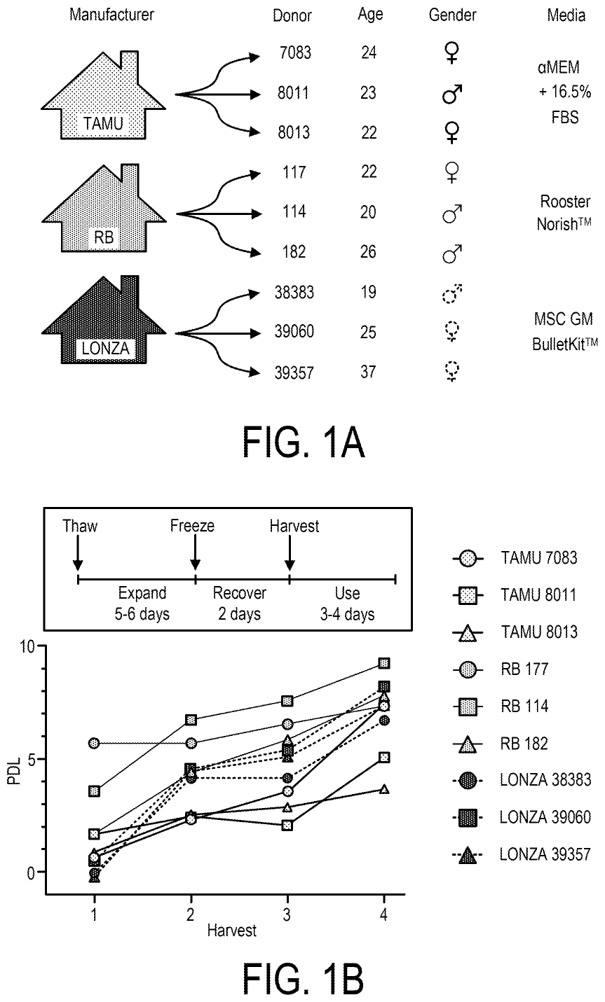

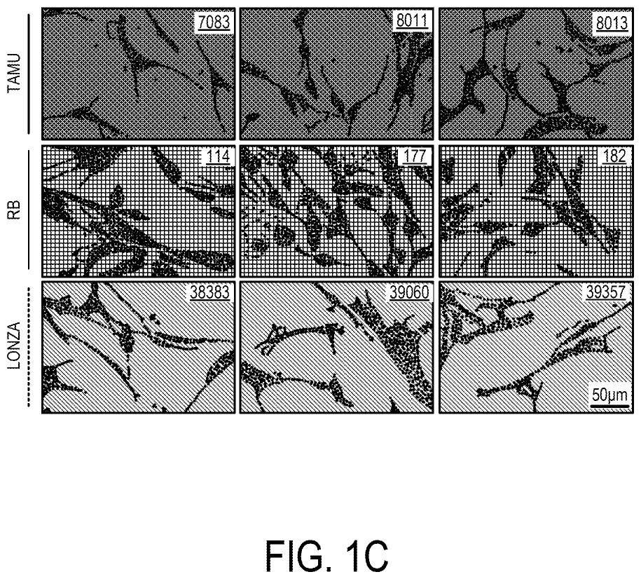

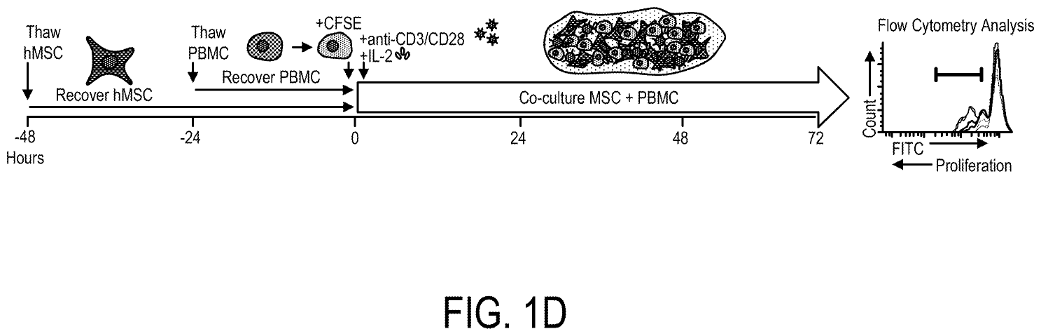

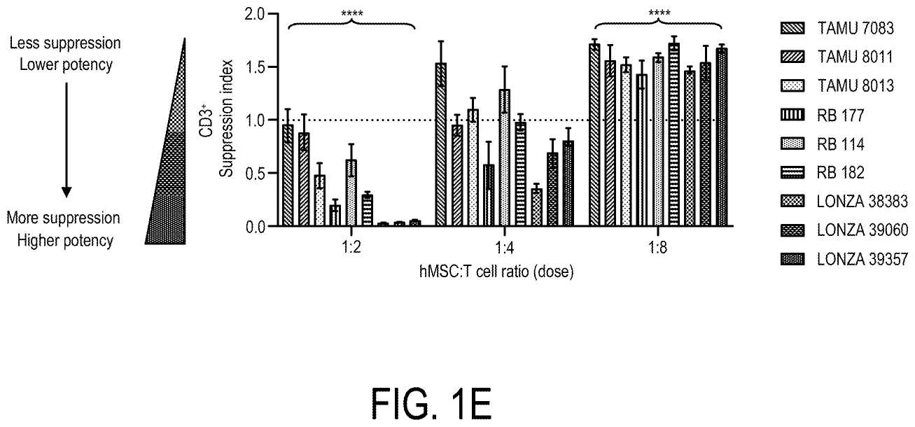

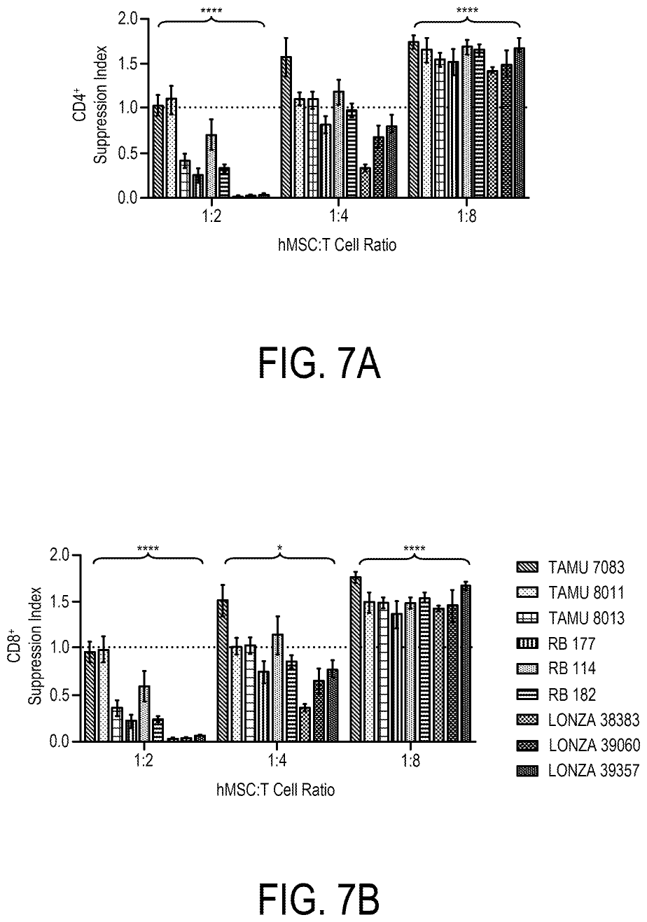

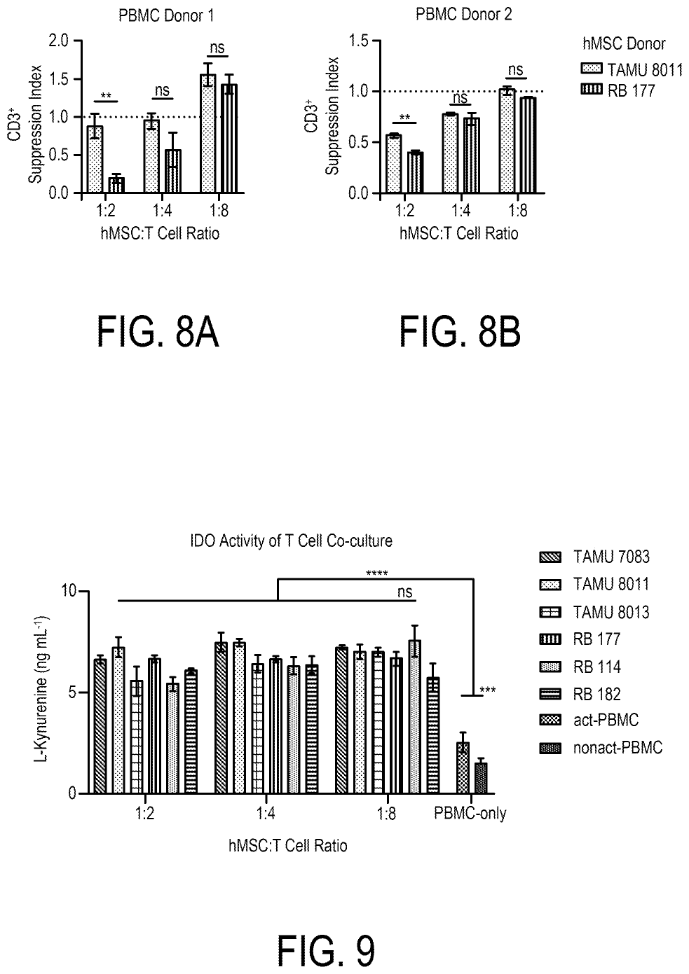

Differences in hMSC donor and manufacturing processes account for variability in product performance. Bone marrow-derived hMSCs were evaluated from nine healthy donors acquired from three manufacturers, and each cultured per the manufacturer's specifications (FIG. 1a). Donor age ranged from 19-37 years old with no clear relationship between donor age or sex and population doubling level (PDL) (FIG. 1b). Distinct morphological features were found among cell lines from different manufacturers but indistinguishable morphologies of donors from the same manufacturer (FIG. 1c). Variability among manufacturers is a consistent trend observed throughout subsequent analyses. Functional hMSC donor potency was assessed by evaluation of CD3⁺ T cell suppression following hMSC:PBMC co-culture, as per recommendations of the International Society for Cellular Therapy (ISCT) (Galipeau, 2016) (FIG. 1d). In this assay, a lower suppression index value corresponds to greater T cell suppression and a more potent cell product. hMSCs suppressed T cell proliferation in a dose-dependent manner with significant suppression for co-cultures at high hMSC dose (1:2 hMSC:T cell) (FIG. 1e). The LONZA cell lines demonstrated the greatest suppression, indicating these cell lines as the most potent. Consistent with other reports, lower hMSC dose (1:8 hMSC:T cell) resulted in increased T cell proliferation compared to activated PBMC-only control (Najar 2016; Le Blanc 2003). Results were consistent across helper CD4⁺ T cell and killer CD8⁺ T cell subpopulations (FIG. 7). Additionally, suppression levels measured with a different PBMC donor showed consistent relative hMSC donor suppression, but disparate absolute suppression levels (FIG. 8), supportive of the metrics use for relative potency correlation (using single PBMC donor) and highlighting limitations of assay scalability.

### IDO and PD-L1 as Potency Metrics in 2D Culture

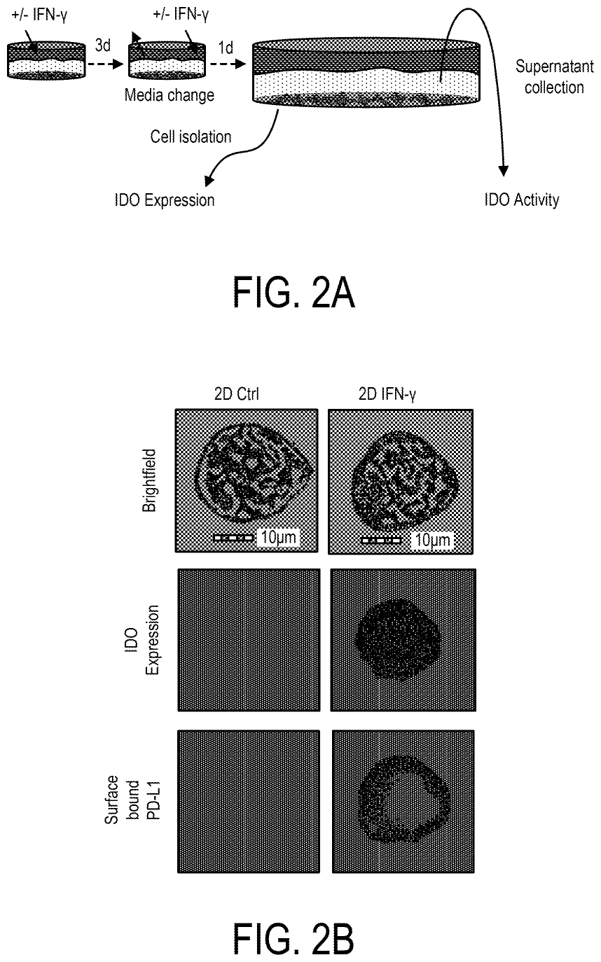

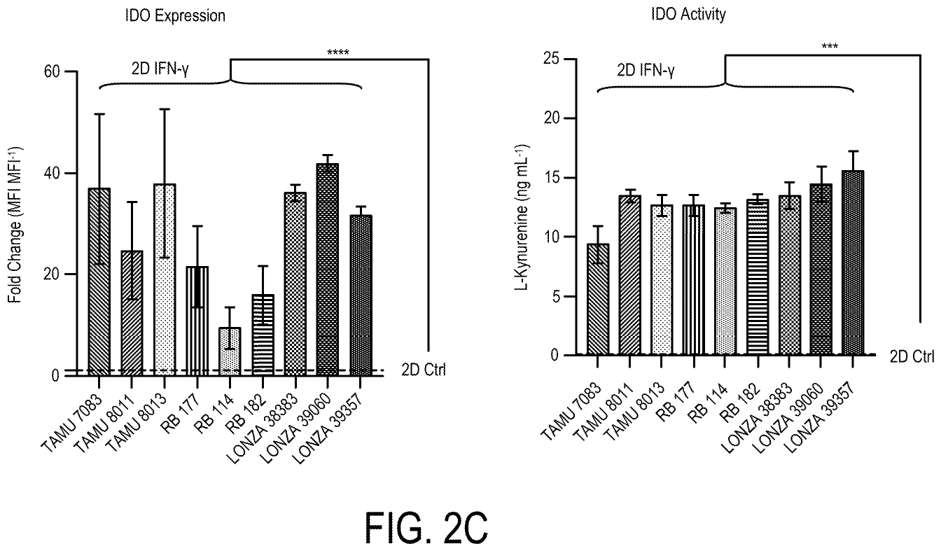

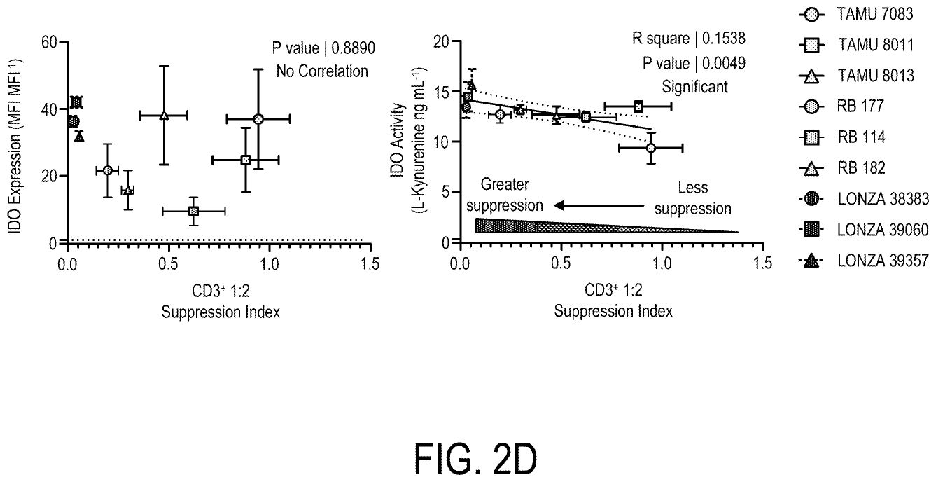

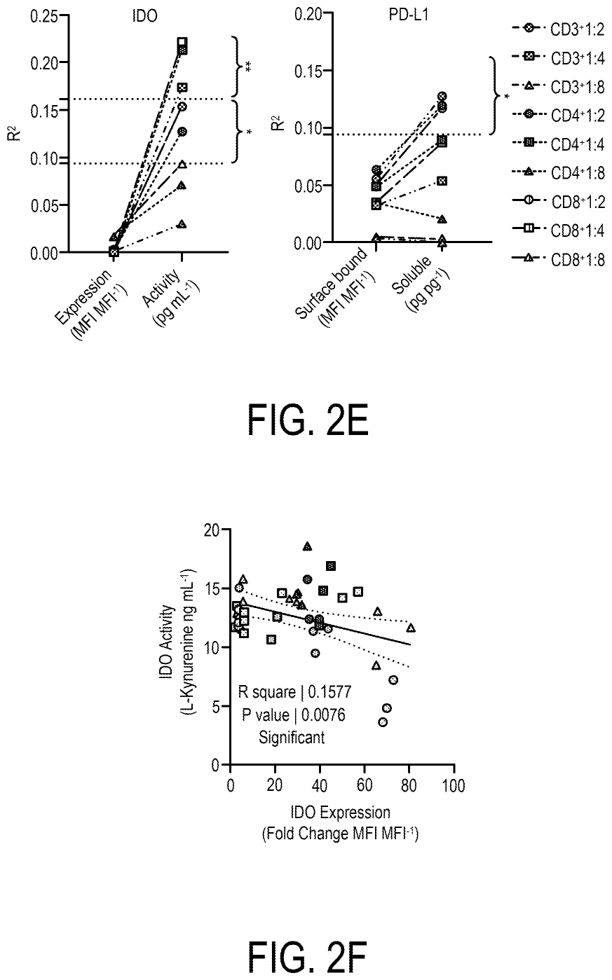

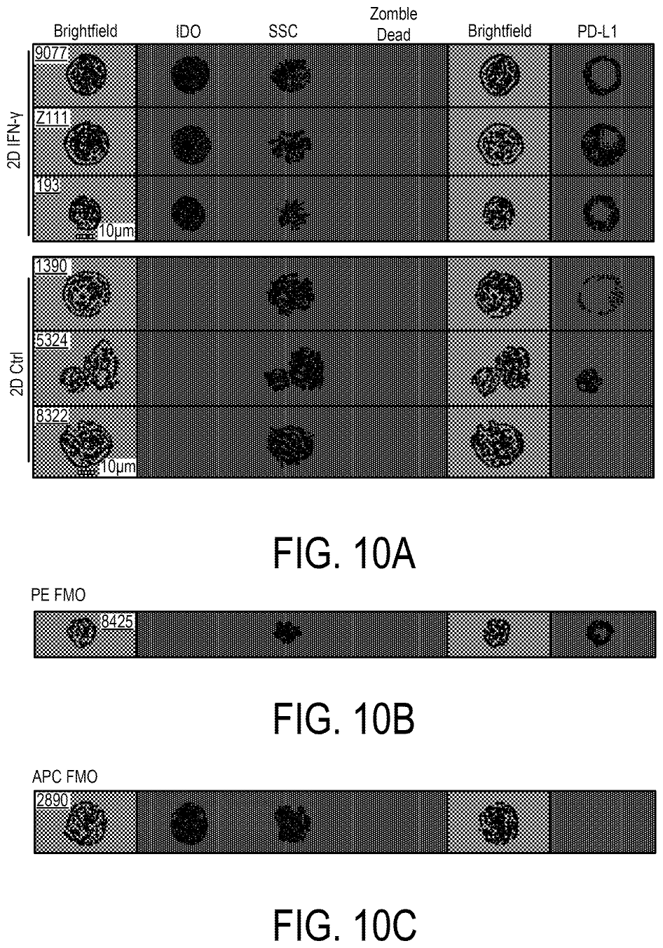

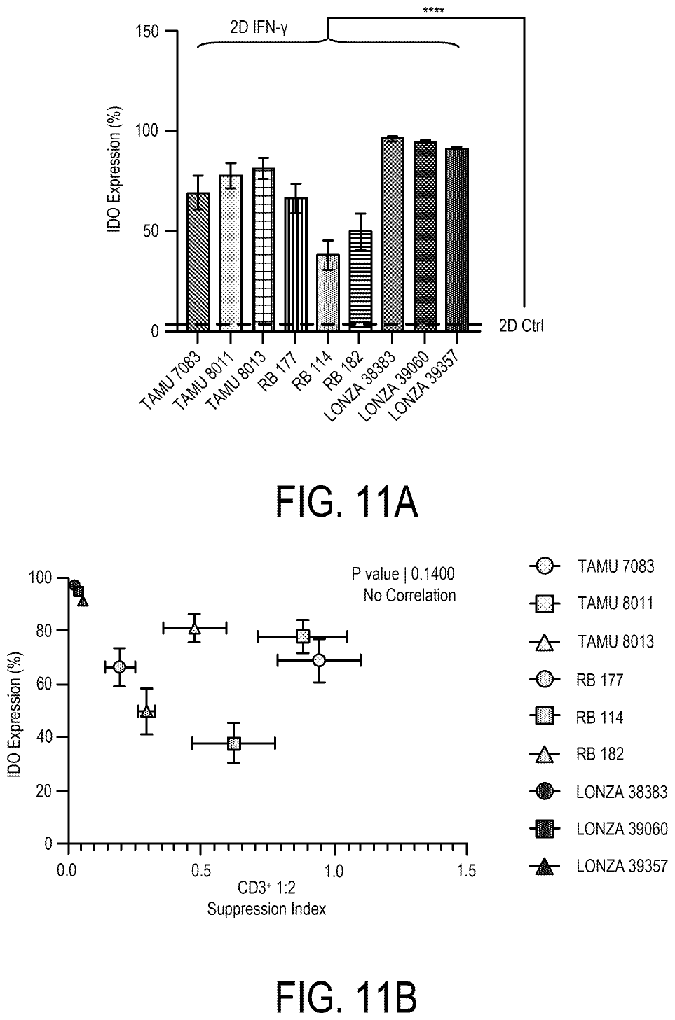

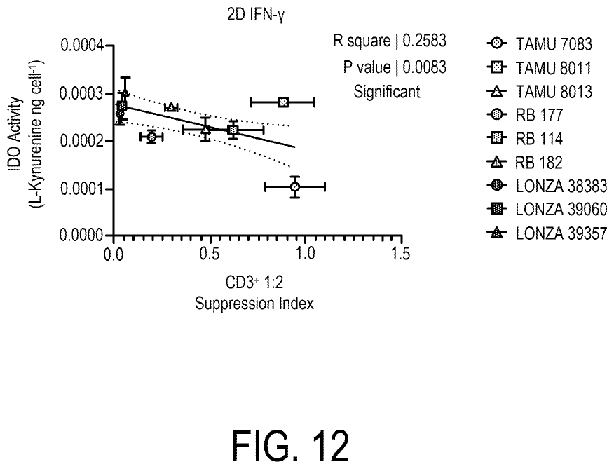

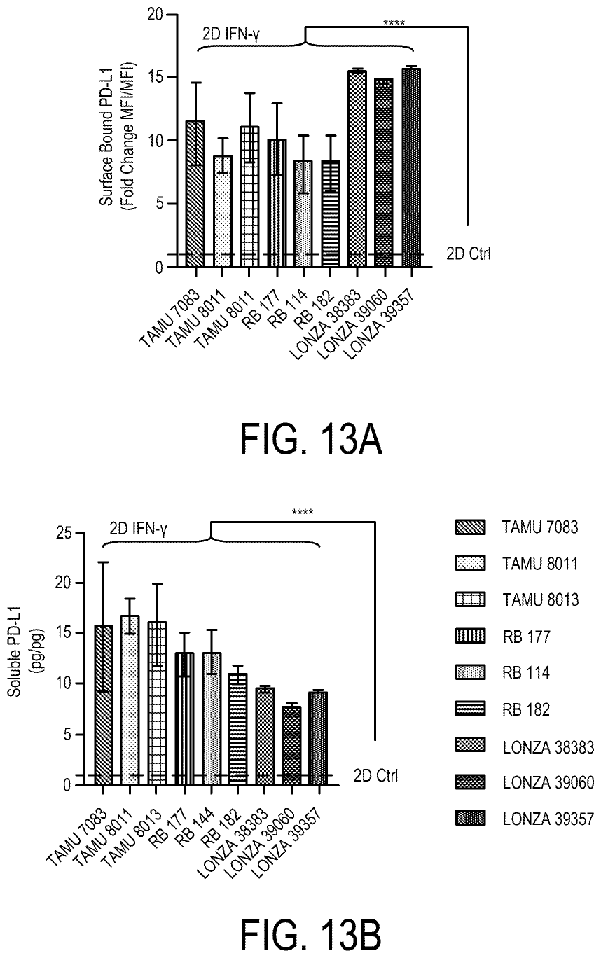

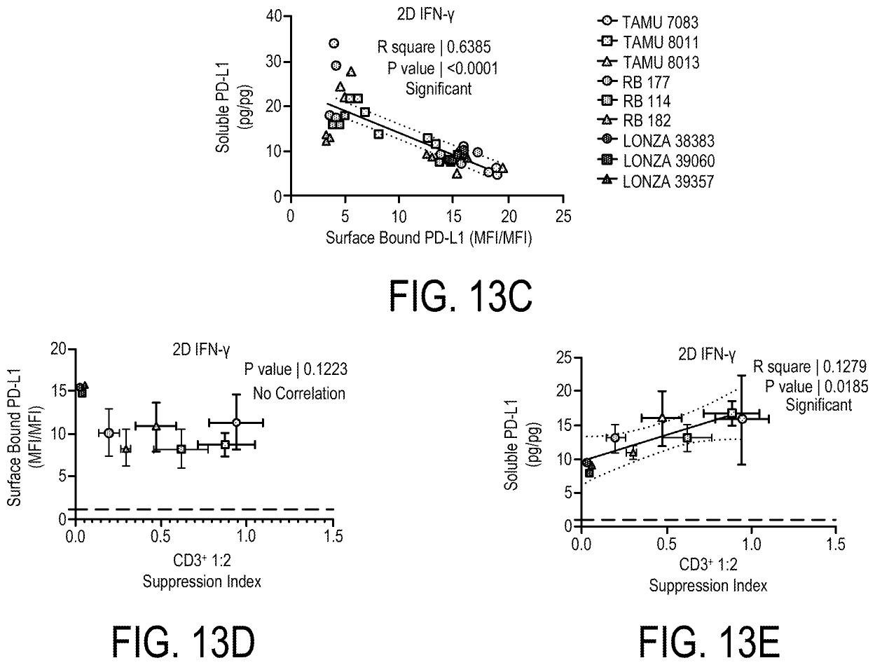

Common surrogate potency markers IDO and PD-L1 (also known as B7-H1) were compared to suppression index for hMSC donors. IDO is an intracellular enzyme that inhibits T cell proliferation by enzymatic catabolism of essential amino acid tryptophan to kynurenine metabolites (Pallotta 2011). The potency utility of IDO was assessed by correlation of both intracellular IDO expression and IDO enzymatic activity (measured as secreted byproduct L-kynurenine) (Agaugue 2006; Braun 2005) to functional donor-matched T cell suppression levels. In accordance with the established role of IDO activity in hMSC-mediated T cell suppression, (Francois 2012) and found elevated IDO activity in hMSC:T cell co-culture supernatant compared to PBMC-only controls, but no difference across hMSC dose (FIG. 9). Although IDO is a widely used surrogate marker of hMSC potency (Levy 2020), intracellular IDO expression and enzymatic IDO activity are often, without merit, used interchangeably as potency metrics. Here, intracellular IDO expression by flow cytometry and secreted IDO activity byproduct were evaluated in 2D culture with IFN-γ stimulation (2D IFN-γ) and 2D culture without IFN-γ stimulation (2D Ctrl) for 9 hMSC donors (FIG. 2a). The intracellular localization of IDO was confirmed using imaging flow cytometry (FIG. 2b, FIG. 10). 2D IFN-γ IDO expression and IDO activity were upregulated compared to 2D Ctrl for all hMSC donors (FIG. 2c). High intra-donor variability of IDO expression (normalized 2D Ctrl, MFI MFI-1) was observed across repeated experimentation, in agreement with prior reports (Francois 2012: Guan 2018) and lower variability for % IDO positive metric (FIG. 11). The potency utility of IDO expression and activity were evaluated by linear regression of donor suppression index and IDO levels (FIG. 2d, % IDO positive: FIG. 11). There was correlation of suppression index and IDO activity; this was maintained for IDO activity normalized to cell count, as often reported (ng cell⁻¹, FIG. 12). In summary, IDO activity but not IDO expression had donor-matched correlation to suppression index for various T cell subsets and hMSC doses (FIG. 2e, Table 1). In further analysis of the relation between 2D IFN-γ IDO expression and activity levels, linear regression analysis revealed a negative relationship between the two metrics (FIG. 2f). This can be attributed to alternative intracellular processes of IDO, specifically its role in intracellular signal transducing for immune regulatory TGF-β pathways (Pallotta 2011). Results were similar for the alternative potency marker PD-L1, where surface bound PD-L1 measurements had no correlation to suppression index, but soluble PD-L1 measured in the supernatant had limited correlation (FIG. 2e, FIG. 13, Table 2). Taken together, these results support a lack of robustness and consistency of 2D IFN-γ IDO and PD-L1 potency metrics, in part due to complex and multifaceted signaling mechanisms.

### Engineered On-Chip Microfluidic Potency Assay

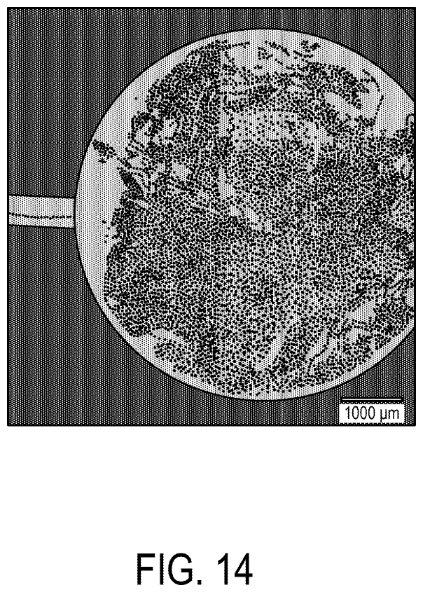

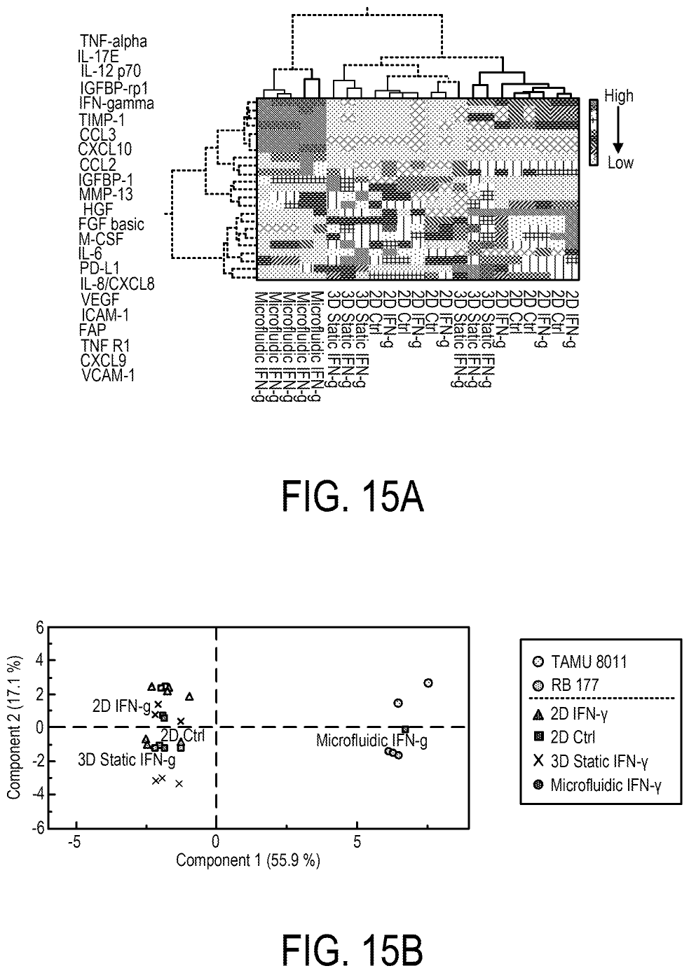

Mechanical, physical, and chemical cues play essential roles in directing cell behavior (Vining, 2017). To address the need for a predictive hMSC potency assay (Levy, 2020, Robb, 2019), a system was engineered with physiologically-relevant mechanical cues and 3D structural support (Low, 2017). It was previously established that hydrogel encapsulation with media-perfusion significantly alters hMSC secretory performance (Williams, 2019). The engineered on-chip 3D microfluidic system encapsulates hMSCs in a synthetic 4-arm maleimide-functionalized poly(ethylene-glycol) (PEG-4MAL) hydrogel cross-linked with dithiolated protease-degradable VPM (GCRDVPMSMRGGDRCG, SEQ ID NO: 1) and non-degradable dithiothreitol (DTT) with presentation of cell-adhesive peptide RGD (GRGDSPC, SEQ ID NO 2) (FIG. 3a). The cell-laden hydrogel is incorporated into a poly(dimethylsiloxane) (PDMS) cast chip and perfused with IFN-γ supplemented media (FIG. 3b). IFN-γ infiltration of the hydrogel was modeled using surrogate FITC-dextran (20 kDa), showing rapid distribution throughout the hydrogel (FIG. 14). Secretory comparison of static and perfused hydrogel cultures as well as 2D controls demonstrates a unique response of perfused microfluidic 3D cultures not captured in static 3D cultures, which had secretory profiles similar to static 2D controls (FIG. 15). It is noted that the driving design goal of the microfluidic design focuses on providing minimal criteria to achieve a predictive hMSC response while maintaining a reproducible and scalable system, rather than striving to accomplish tissue-mimicry (Low, 2017) (Chou, 2020). The microfluidic system was scaled such that up to 40 devices could be run in parallel and maintained a low-cost. The media effluent is easily accessible from the collection syringe, and cell secretome analysis can be nondestructively and temporally performed. This technology was envisioned to be readily incorporated as an in-line assay of current cell therapy manufacturing practices for stepwise potency evaluation.

In pursuit of a predictive system, the microfluidic chip design and analysis was optimized via iterative in vitro-in vivo experimentation to identify analytes secreted following hMSC in vivo delivery and incrementally designed the in vitro system to best capture this in vivo secretory profile (FIG. 3c). For in vivo studies, hMSC-laden hydrogels were polymerized in situ in the subcutaneous space of immunocompromised NSG mice, and 3 days post-injection, the hMSC-laden hydrogel and local tissue were collected and digested for human analyte analysis by multiplex Luminex.

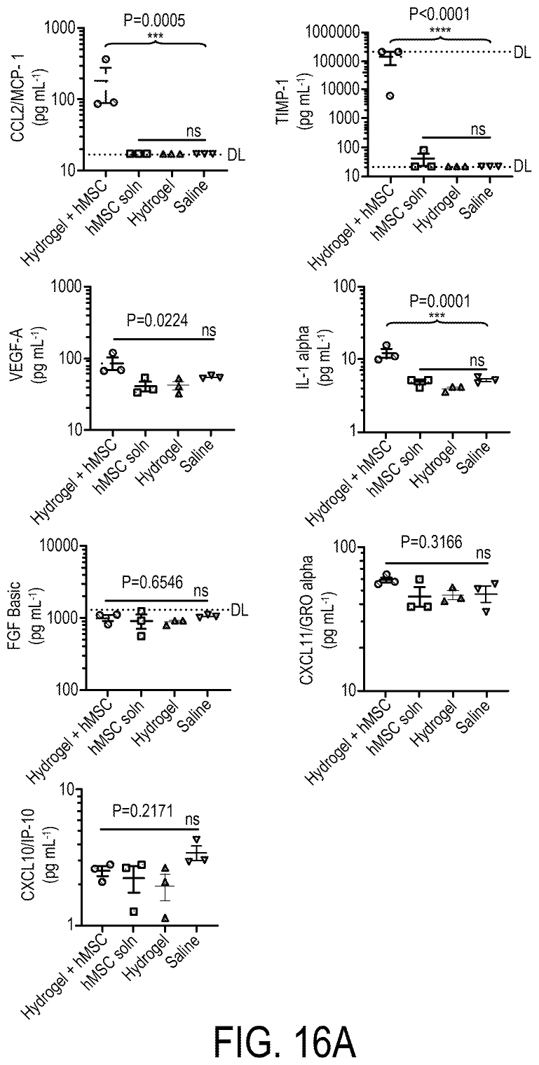

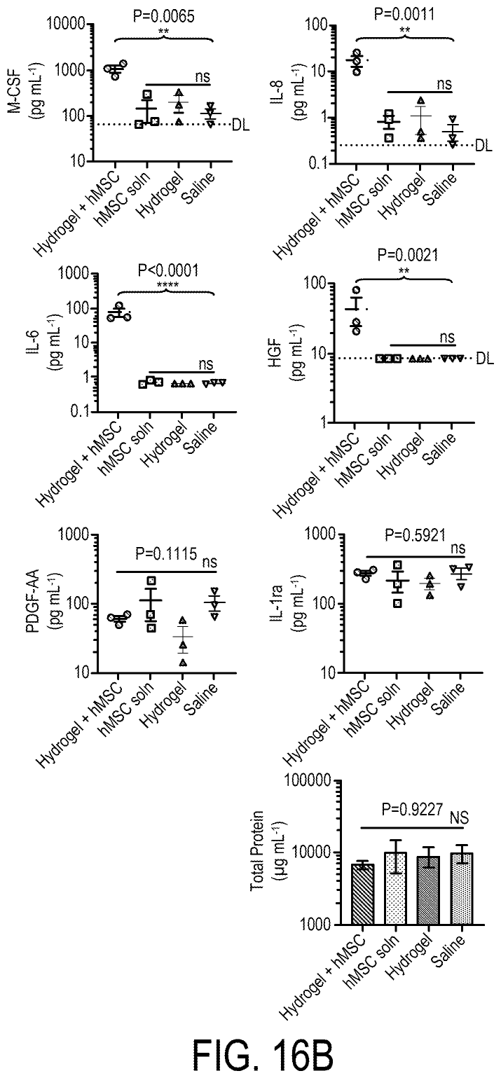

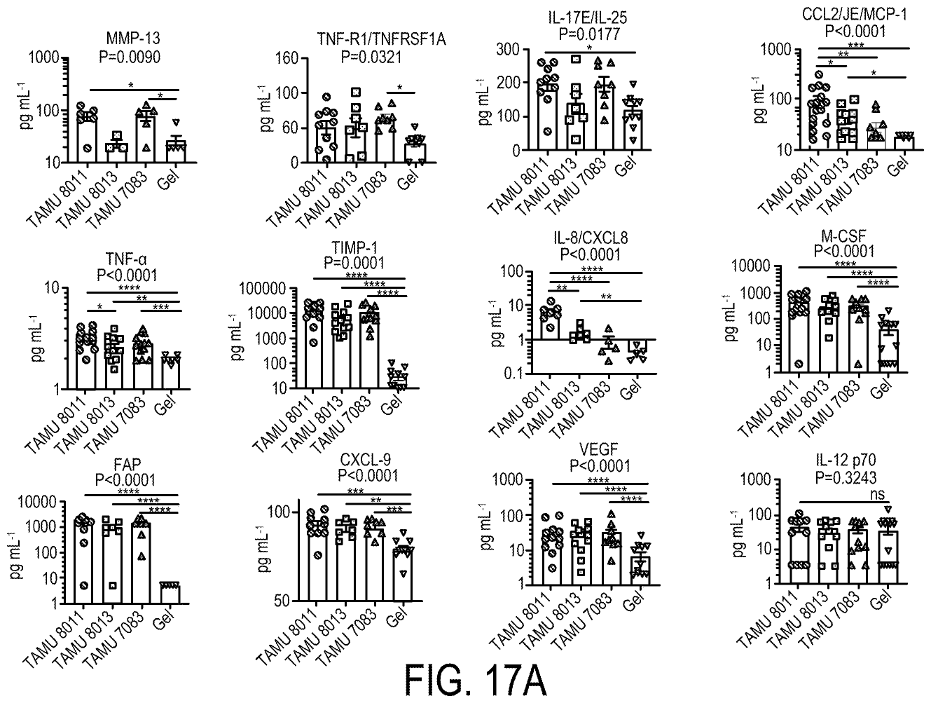

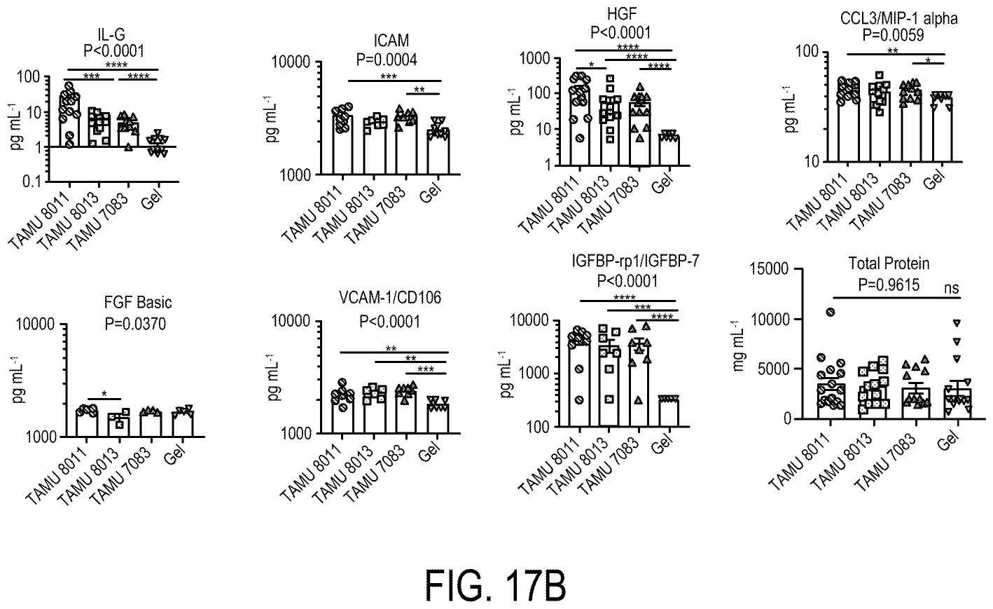

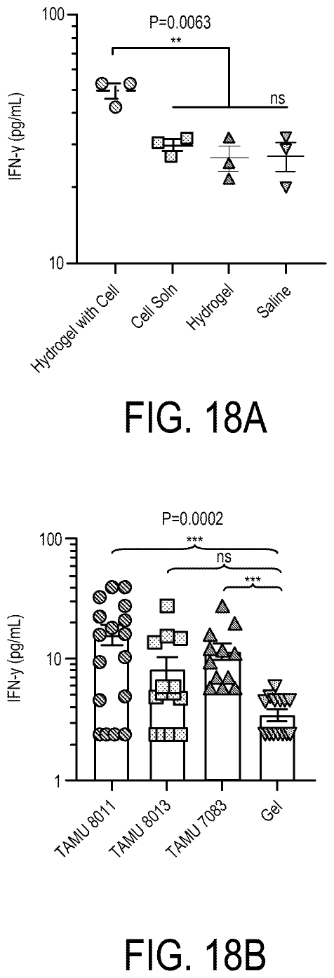

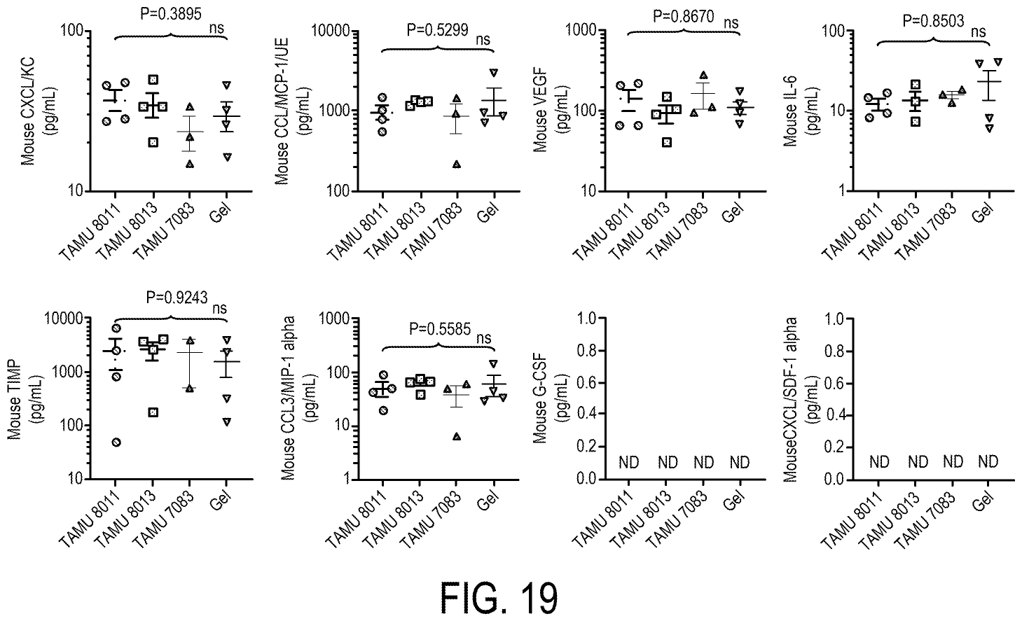

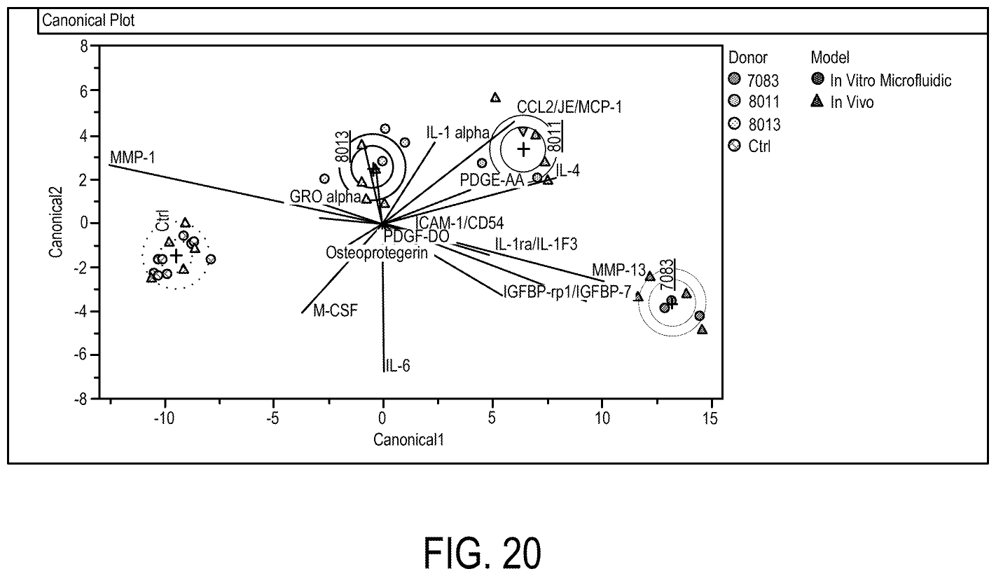

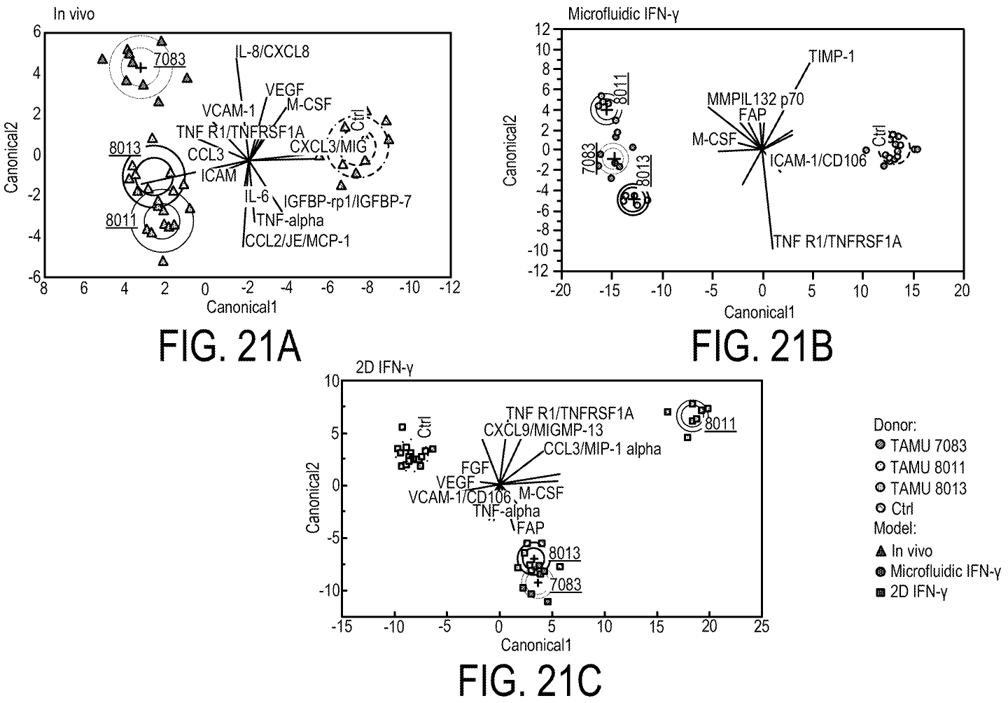

Importantly, prior conclusions drawn from murine pre-clinical models of hMSC have failed to translate to human clinical outcomes (Galipeau, 2018). The goal of the in vivo model used (subcutaneous implantation into immunocompromised mice) is to obtain measurements of hMSC cytokine secretion in an in vivo environment. It is noted that this model is not intended to represent a pathophysiological site and/or disease model nor to evaluate hMSC and immune cell interactions. Initial in vivo studies for microfluidic design optimization showed increased analyte levels of hMSC-laden hydrogels compared to cell-free hydrogel, hMSC suspension (no hydrogel), and saline (FIG. 16), demonstrating hydrogel delivery is necessary to capture local hMSC in vivo responses. Subsequent experimentation incorporated additional donors (TAMU 8011, 8013, 7083) for in vivo secretory analysis (FIG. 17). Notably, an increase of local IFN-γ was observed in hMSC-laden hydrogels compared to controls (FIG. 18), demonstrating the presence of this potent signaling molecule for both in vitro (exogenously supplied) and in vivo (hMSC produced) models. There was no increase in host (mouse) cytokine levels compared to controls, indicative of nominal innate inflammatory response at the hydrogel site and supportive of hMSC (human) analyte origin (FIG. 19). Analytes were selected for further analysis based on either in vivo upregulation or in vivo differences among hMSC donors. Over 75 analytes were screened; analytes of interest were chosen using single and multivariate approaches (FIG. 17, 20). In response to in vivo outcomes, in vitro microfluidic design parameters (flow rate, hydrogel size, stimulation treatment) were optimized for matched in vivo-in vitro secretory responses. This iteration enabled a microfluidic system design and accompanying 20-plex analyte panel with similar in vitro microfluidic and in vivo secretory performance compared to traditional 2D IFN-γ culture (FIG. 21, panel list: Table 3). This targeted approach to develop an hMSC potency assay addresses translational limitations of current 2D potency assays by improving on the physiological relevance of hMSC stimulation and marker analysis.

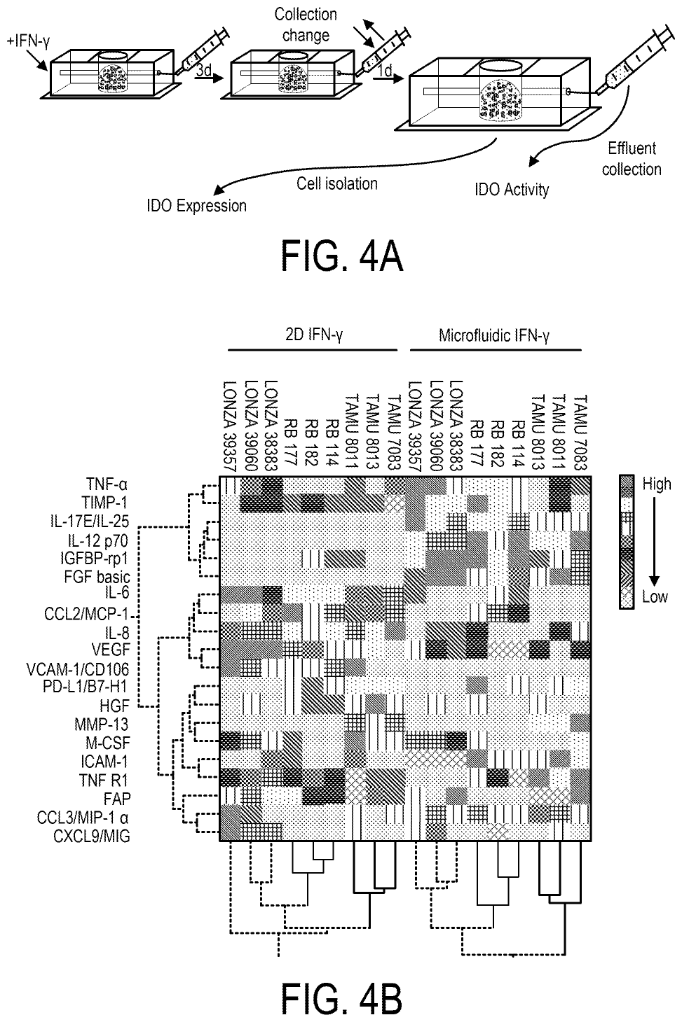

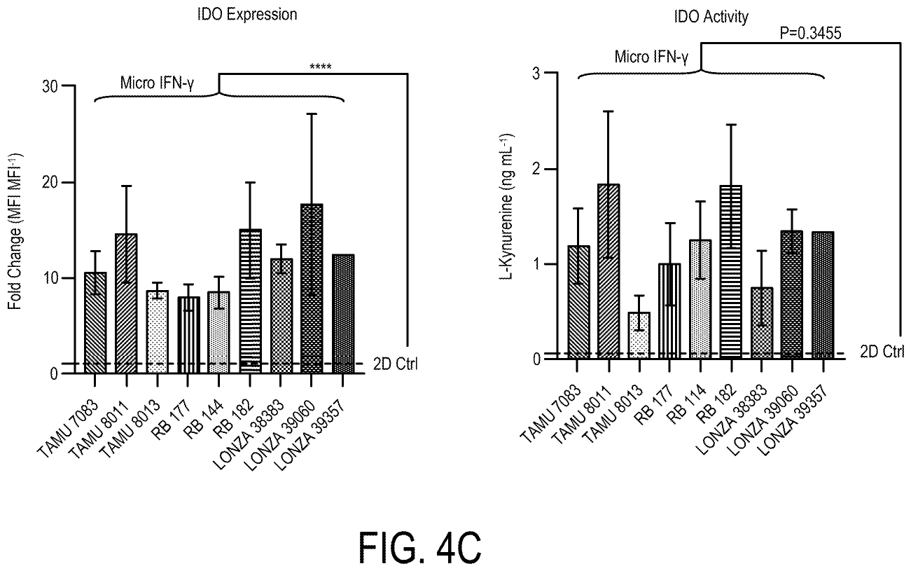

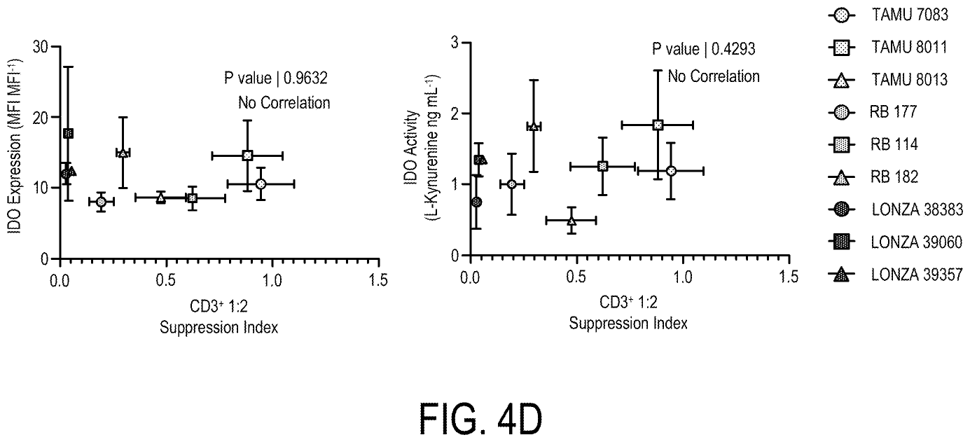

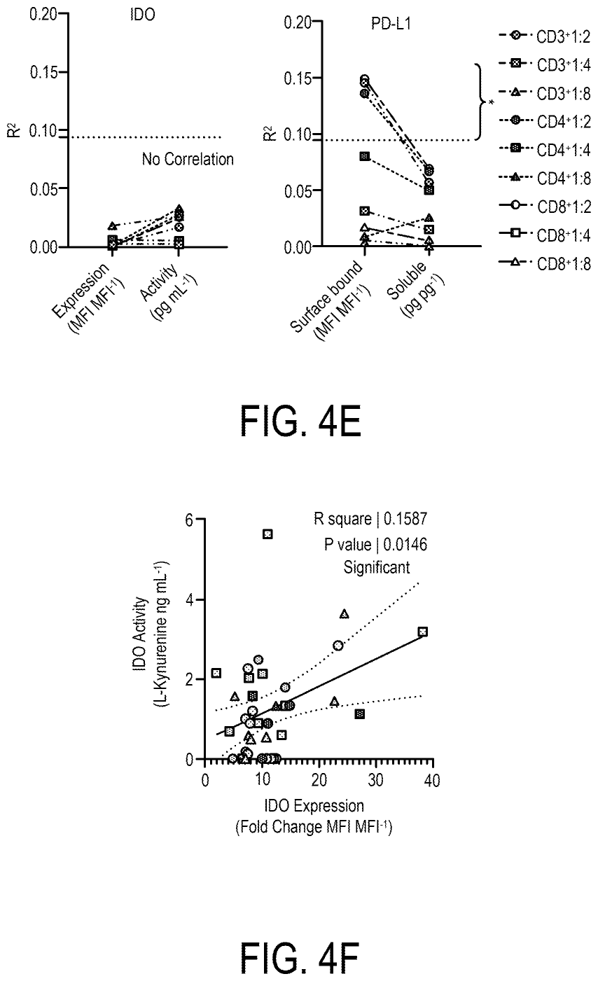

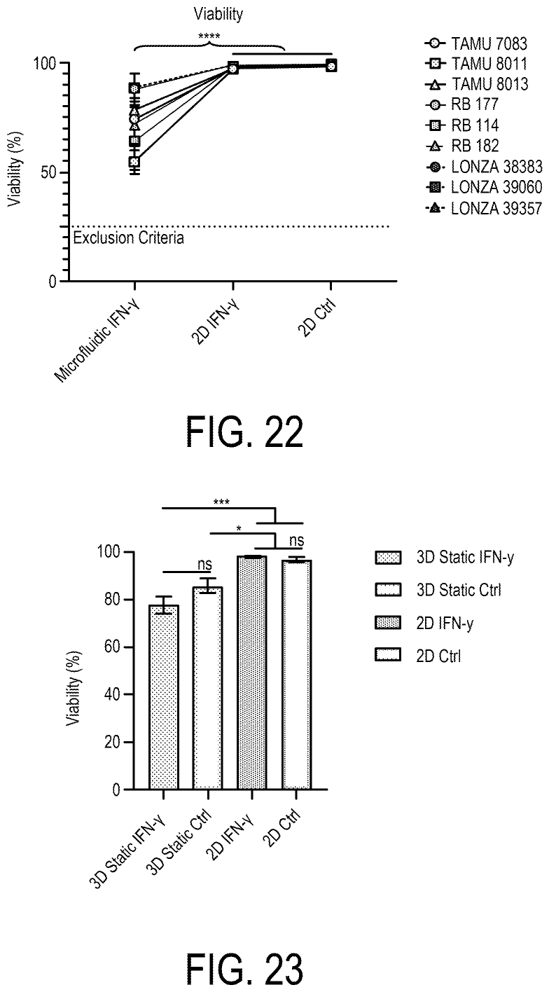

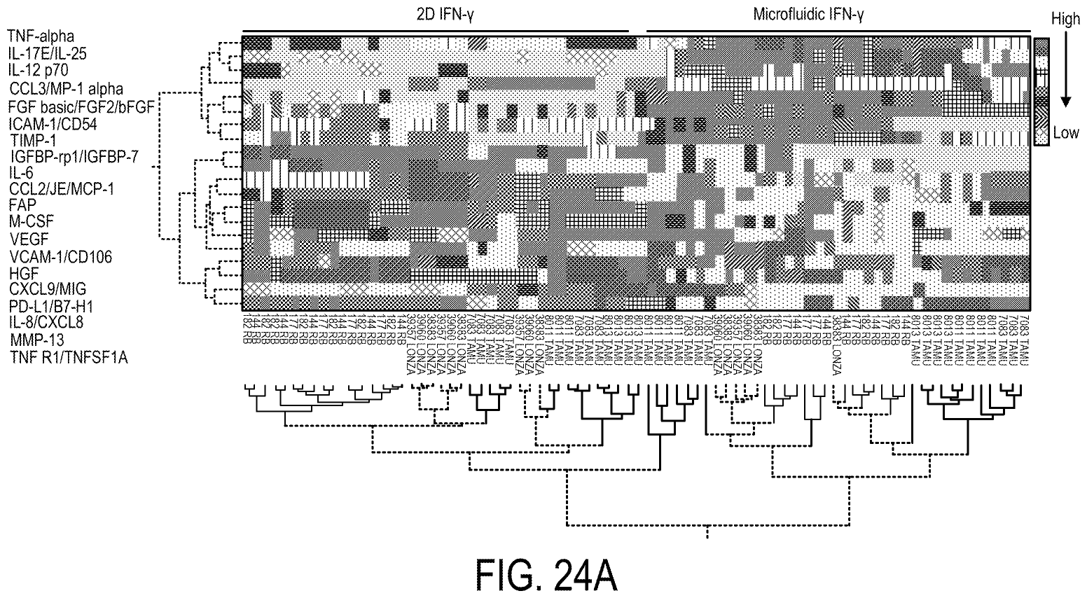

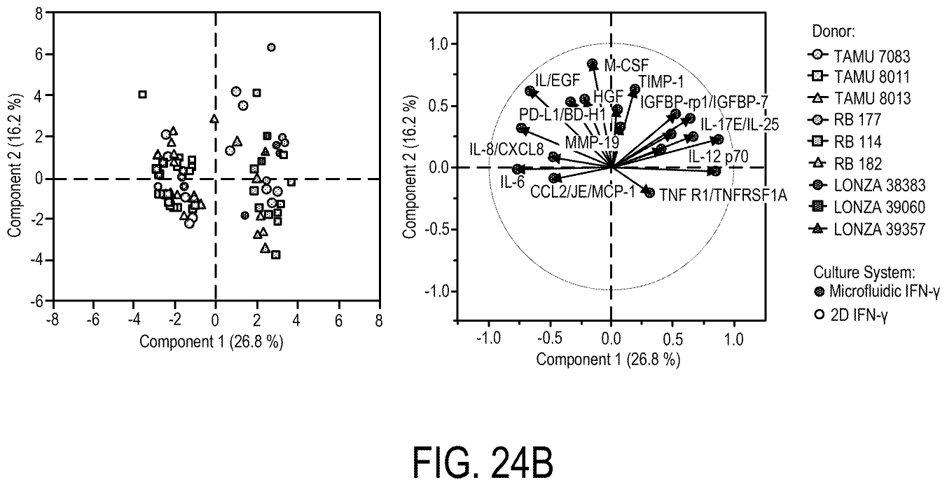

On-chip microfluidic culture was run alongside 2D experiments with continuous perfusion of IFN-γ supplemented media (microfluidic IFN-γ). At study completion, secretome analysis was performed on collected media effluent via Luminex assay and cells were recovered following hydrogel degradation and analyzed by flow cytometry (FIG. 4a). Cell viability was reduced in the microfluidic IFN-γ culture compared to 2D IFN-γ and 2D Ctrl (microfluidic IFN-γ=70.9±4.2%, 2D IFN-γ=98.0±0.24%, 2D Ctrl=98.7±0.10%; FIG. 22). Viability was partially restored when hMSC-laden hydrogels were cultured without perfusion and in a media bath (3D Static IFN-γ=77.5±3.6%, 3D Static Ctrl=85.8±2.9%; FIG. 23). It was posited that this difference in viability between 3D-encapsulated and 2D cultures is biased by the 2D culture removal of non-adhered dead cells in the cell supernatant. Alternatively, the partial improvement in viability of static 3D controls over perfused 3D systems may be a consequence of shear forces, which have been reported to promote cell death (Tanzeglock, 2009). Secretome analysis between systems revealed clear separation between microfluidic IFN-γ and 2D IFN-γ cultures (FIG. 4b, FIG. 24), emphasizing the influence of culture system on hMSC secretory response and the need for precisely defined culture environments in potency assay development.

### Evaluation of Microfluidic IFN-γ IDO and PD-L1

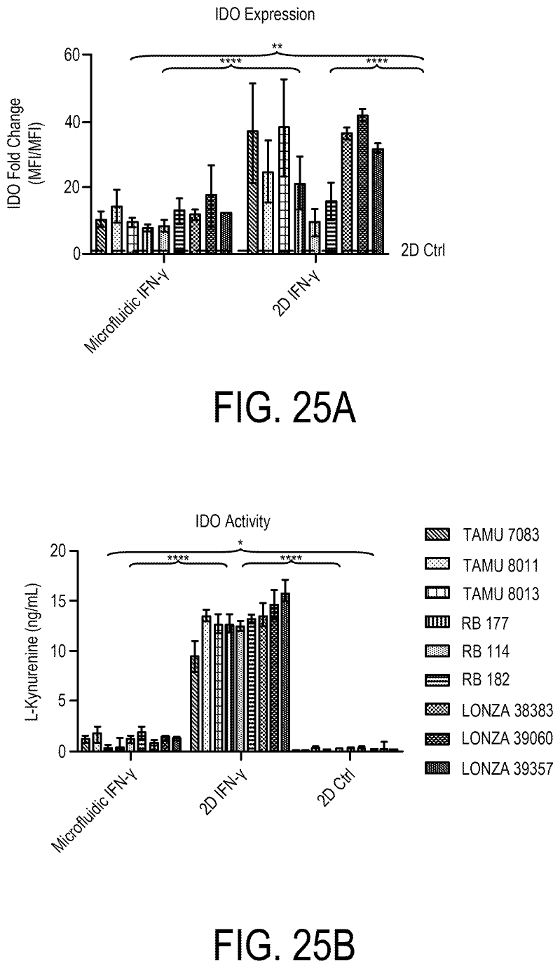

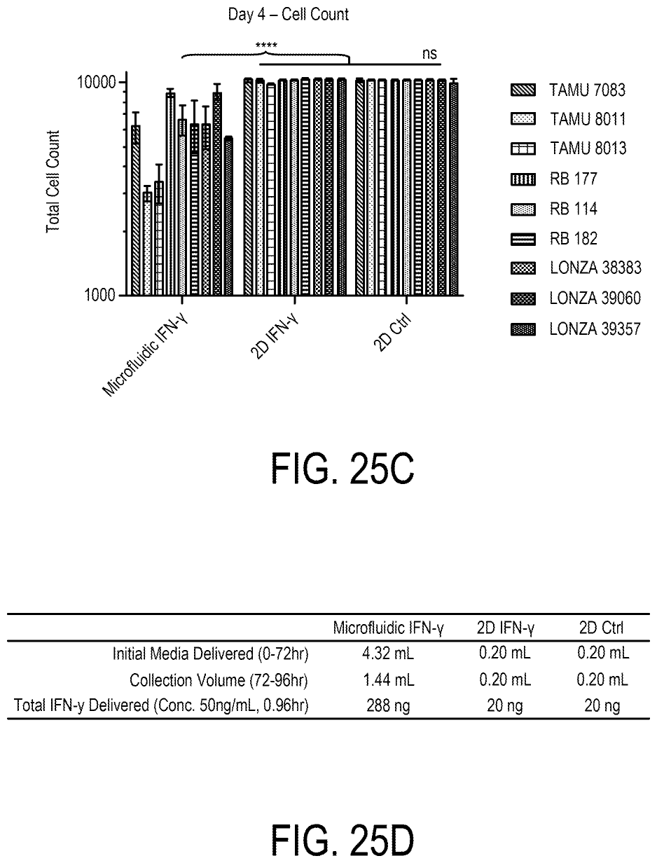

To evaluate hMSC IDO potency utility in microfluidic culture, paralleled IDO analysis was performed as previously presented for 2D culture. Microfluidic IFN-γ IDO expression but not IDO activity were upregulated compared to 2D Ctrl (FIG. 4c). Although IDO expression and IDO activity in 2D IFN-γ culture exceeded microfluidic IFN-γ levels (FIG. 25), the different culture systems introduce inherent experimental design variation, limiting the value of direct system comparison.

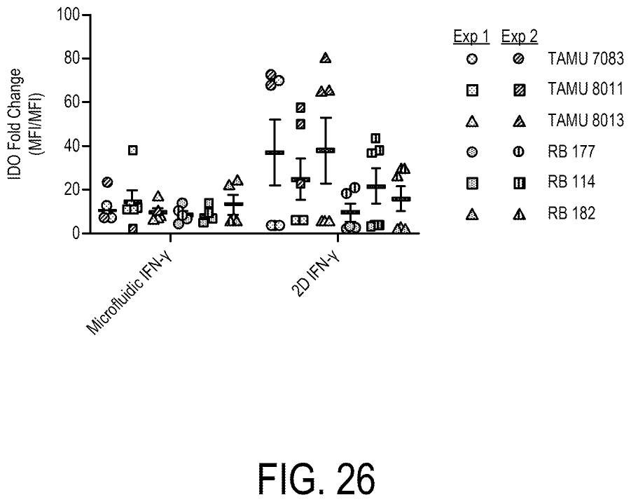

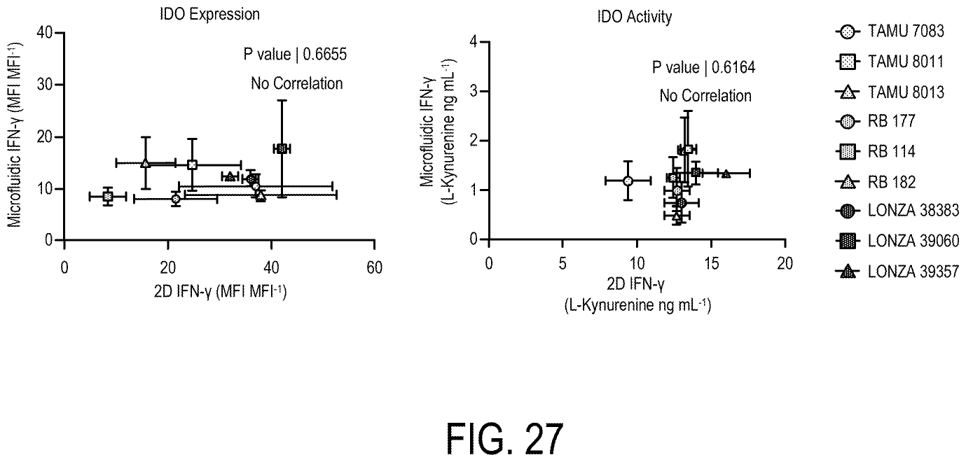

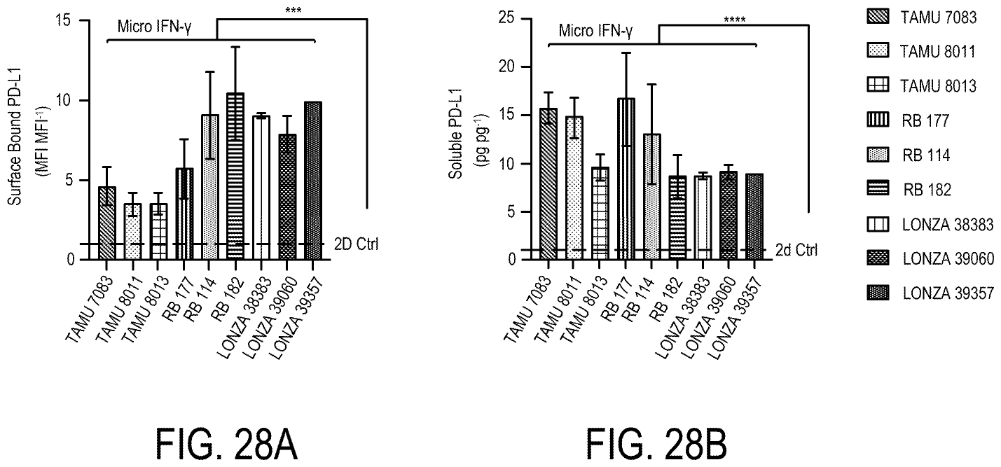

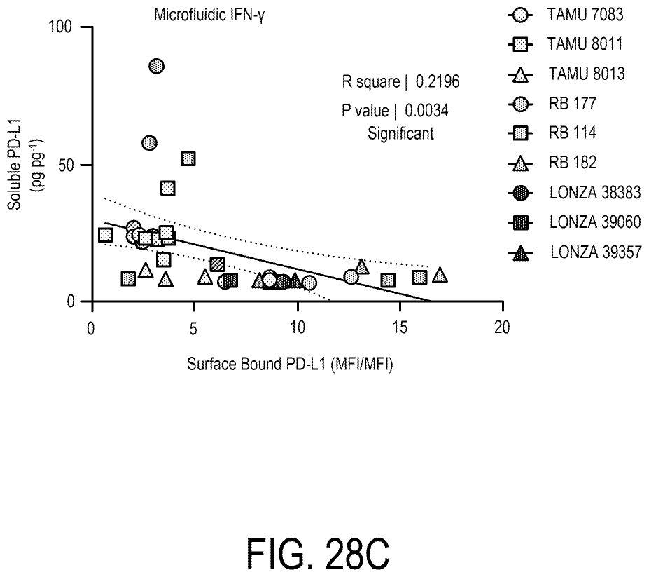

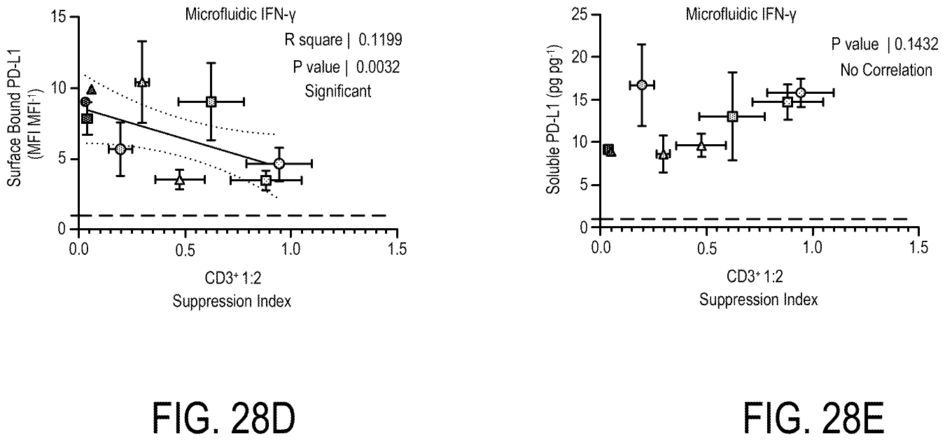

The experimental reproducibility of donor-specific IDO expression levels was improved in microfluidic IFN-γ cultures compared to 2D IFN-γ (FIG. 26). Linear regression analysis of donor-matched suppression index values and IDO expression and activity showed a lack of potency utility in microfluidic IFN-γ culture (FIG. 4d, e). Interestingly, in contrast to 2D IFN-γ culture, IDO expression and IDO activity were positively correlated in the microfluidic IFN-γ system (FIG. 4f), indicating differential IDO regulation across 2D and microfluidic systems. Comparison of donor-matched IDO between 2D IFN-γ and microfluidic IFN-γ cultures showed a lack of correlation, further supporting culture-dependent differences in IDO regulation (FIG. 27). Again, results were similar for additional potency marker PD-L1, where surface bound PD-L1 had limited correlation to suppression index and soluble PD-L1 was non-correlative for each hMSC dose and T cell subset (FIG. 4e, FIG. 28). This is in contrast to the 2D result where soluble PD-L1, but not surface bound, had suppression index correlation (FIG. 28). In general, potency analysis for IDO and PD-L1 metrics demonstrated inconsistent and generally poor correlative confidence across both culture systems (Table 1, 2). The inconsistent trends of IDO and PD-L1 metrics across both 2D IFN-γ and microfluidic IFN-γ culture systems highlight their limited utility as hMSC surrogate potency markers.

### Multiplex Secretome Analysis for Potency Evaluation

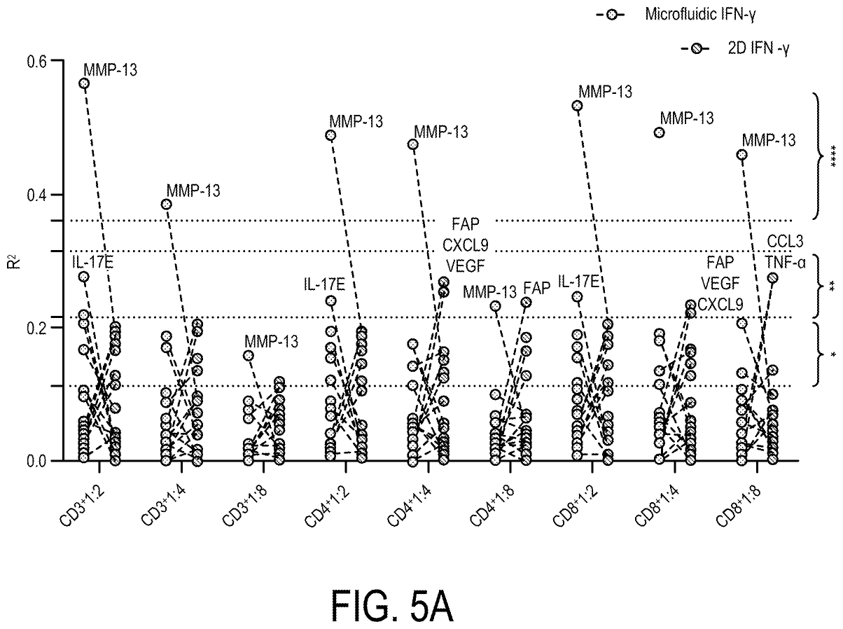

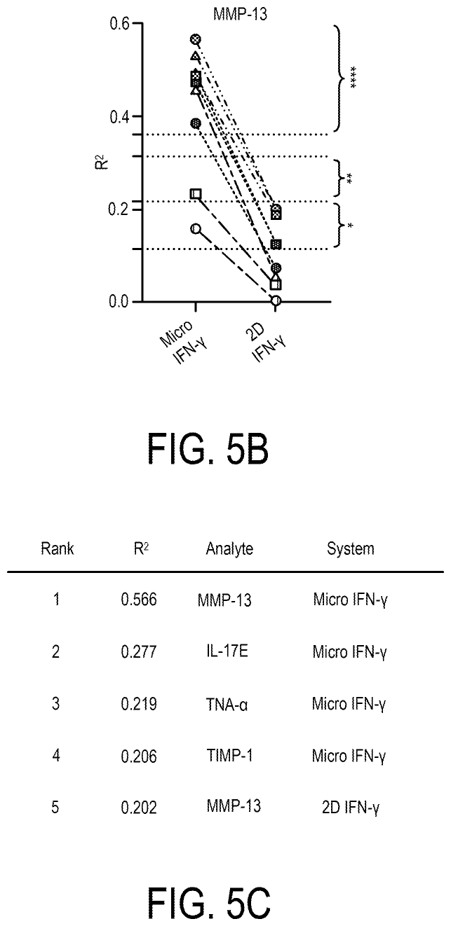

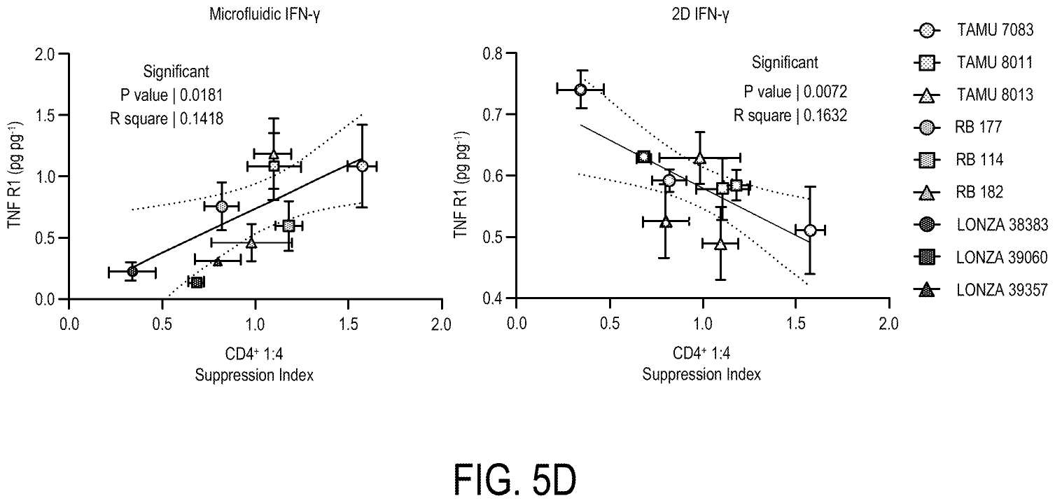

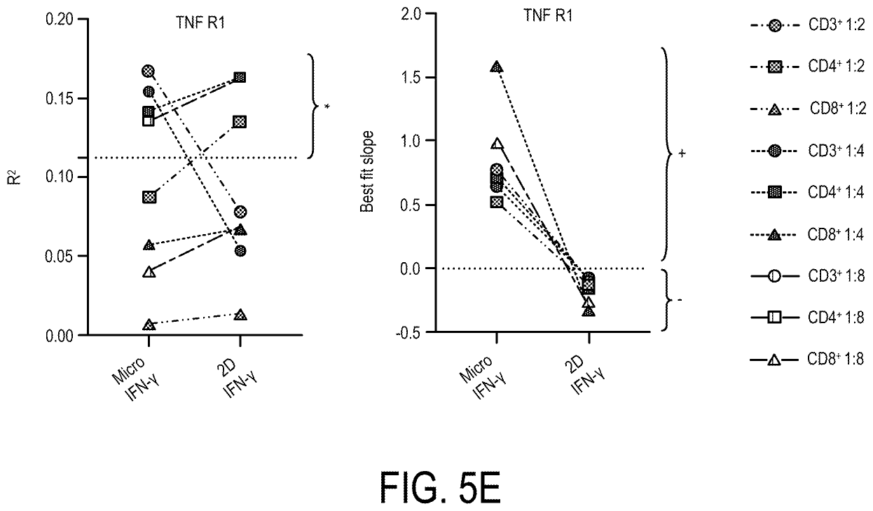

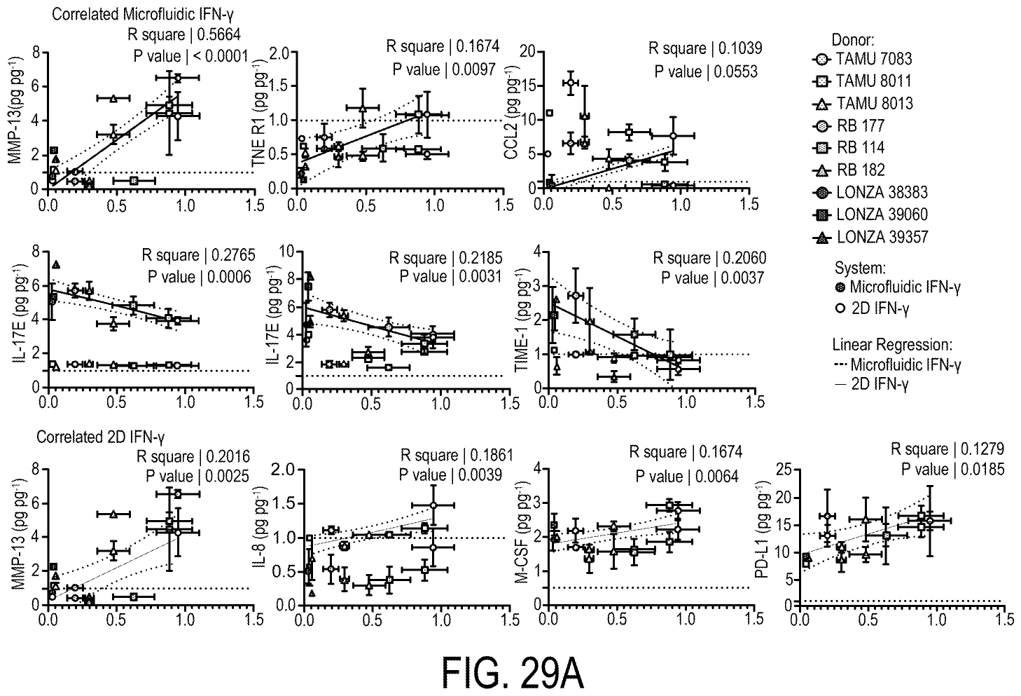

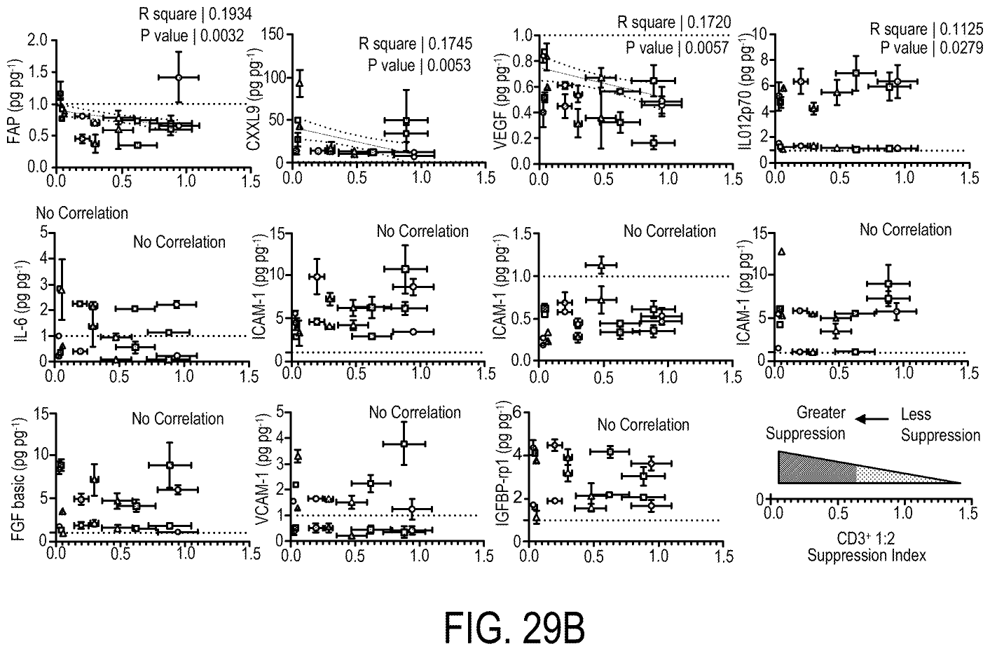

Various other hMSC secreted proteins have been proposed as surrogate potency markers (Galipeau, 2016). To assess the functional potency utility of the 20-plex analyte panel informed by the hMSC-delivered in vivo model, linear regression analysis was performed on donor-matched T cell suppression index against analyte secretion levels (normalized 2D Ctrl, pg pg⁻¹) for microfluidic IFN-γ and 2D IFN-γ stimulated cultures (FIG. 29, Table 4, 5). Of the 20 analytes assessed for functional potency correlation, matrix metalloproteinase 13 (MMP-13) in microfluidic IFN-γ culture system significantly outperformed all other metrics (FIG. 5a). Therapeutically, MMP-13 is an enzymatic driver of articular cartilage degradation and is often used as a marker of disease progression in osteoarthritis (Malemud, 2019) (Wang, 2013). Additionally, MMP-13 inhibitors are promising targets for arthritic therapeutic intervention (Hu, 2021). In both microfluidic IFN-γ and 2D IFN-γ systems, lower MMP-13 secretion correlated to more potent hMSC donor lines (FIG. 29). In the microfluidic IFN-γ system, this correlation was maintained for each hMSC dose and T cell subset (FIG. 5b).

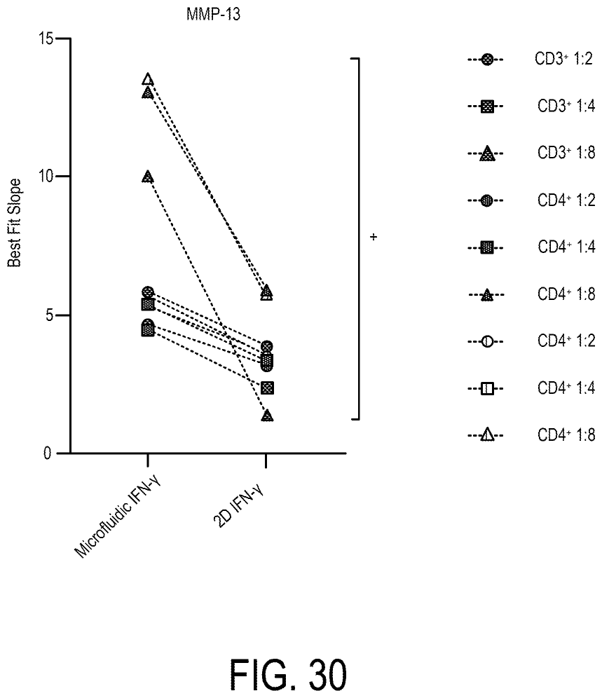

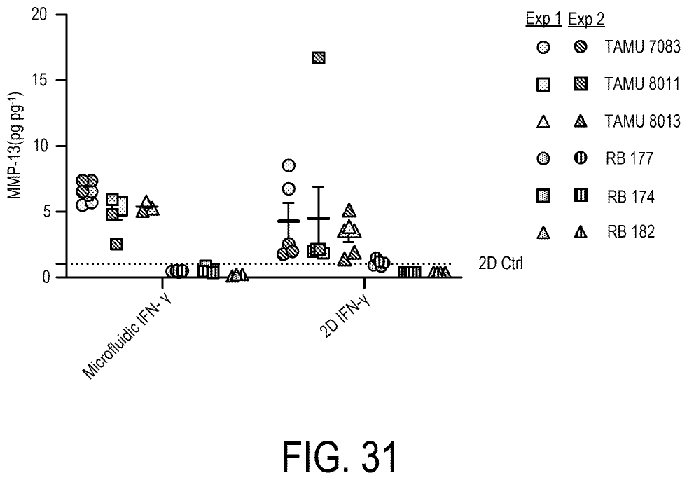

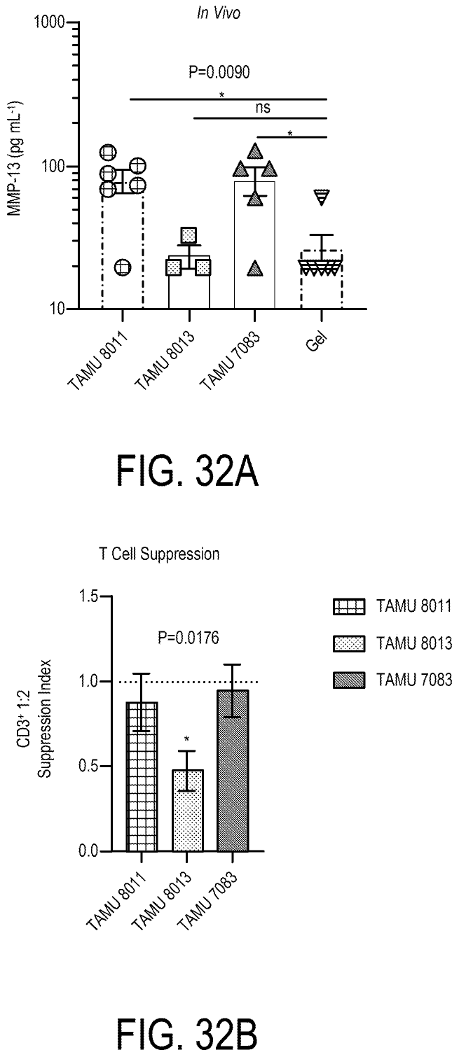

In comparison to 2D IFN-γ, MMP-13 secretion in the microfluidic IFN-γ system offered improved linear fit R² to functional potency metrics (FIG. 5b), increased best-fit slopes (FIG. 30), and high experimental reproducibility (FIG. 31), all important for development of a robust potency assay. Additionally, the in vitro functional suppression trends were in agreement with in vivo MMP-13 secretion levels (FIG. 32).

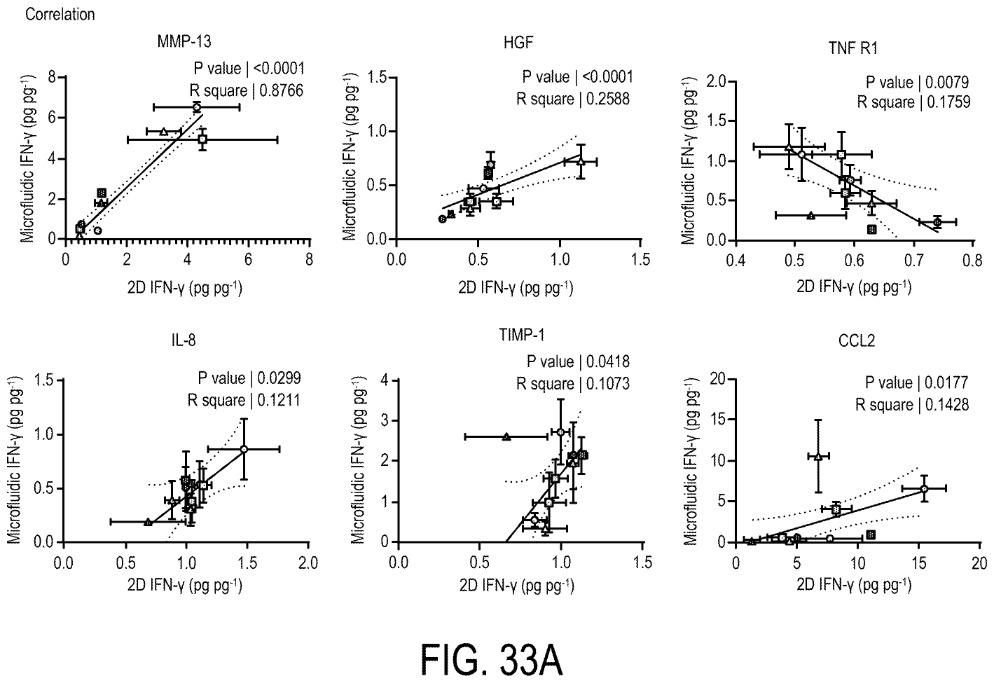

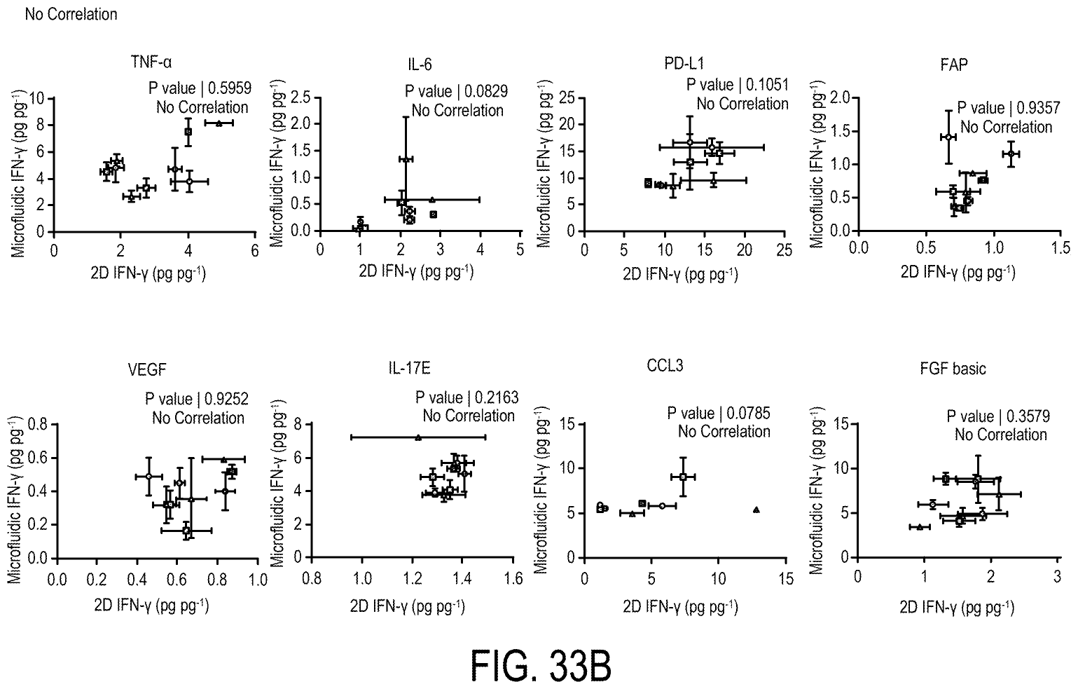

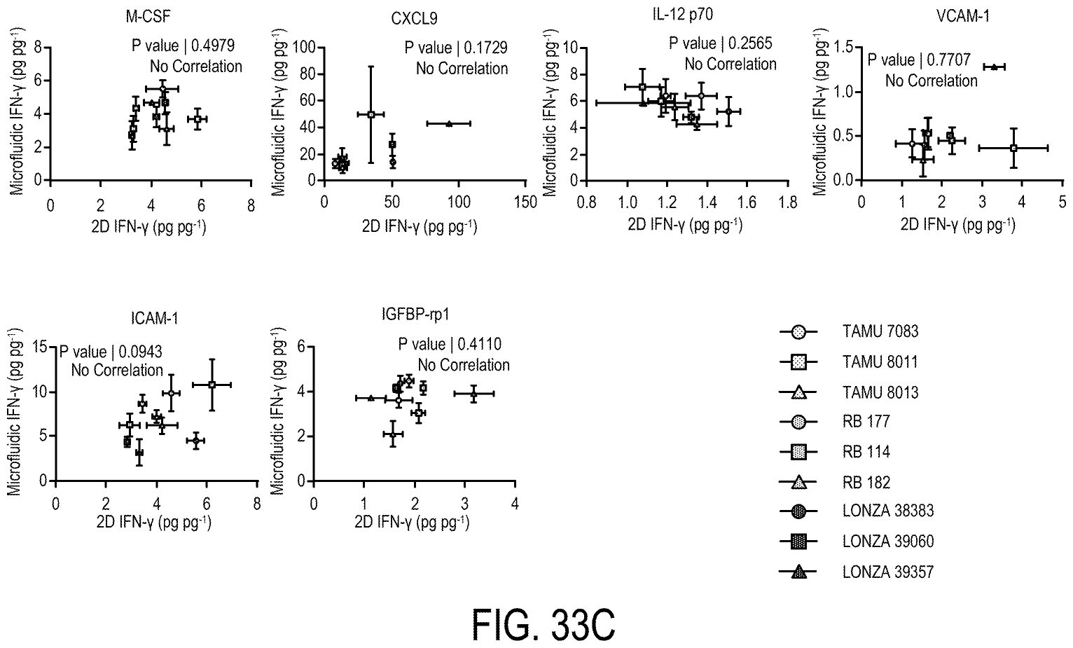

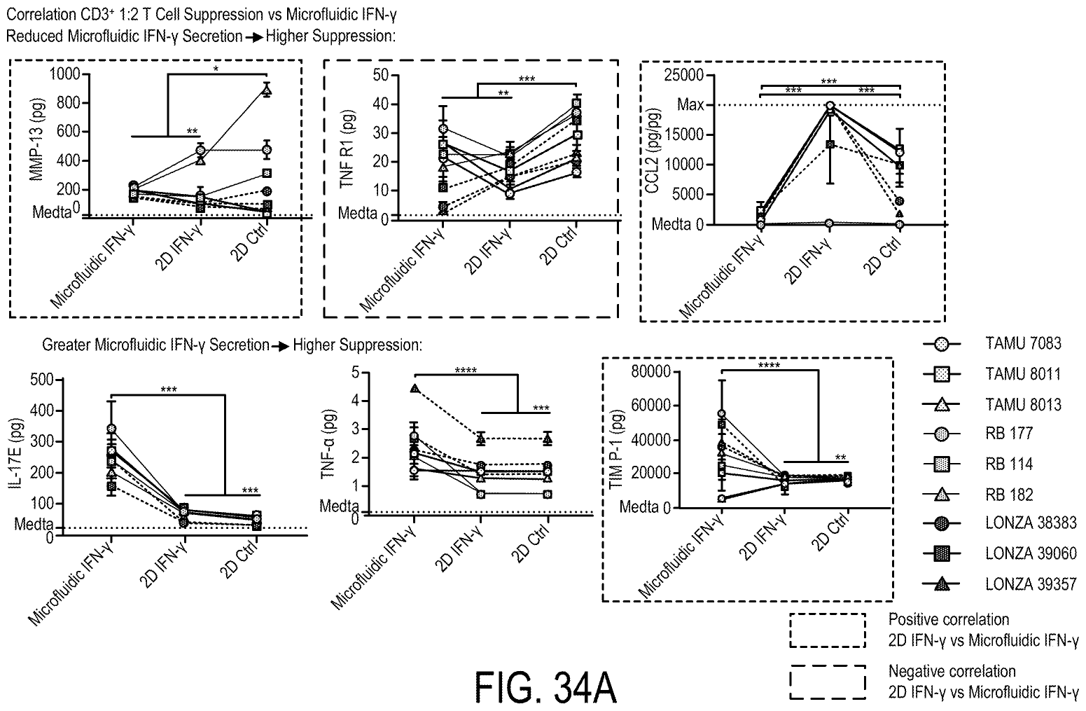

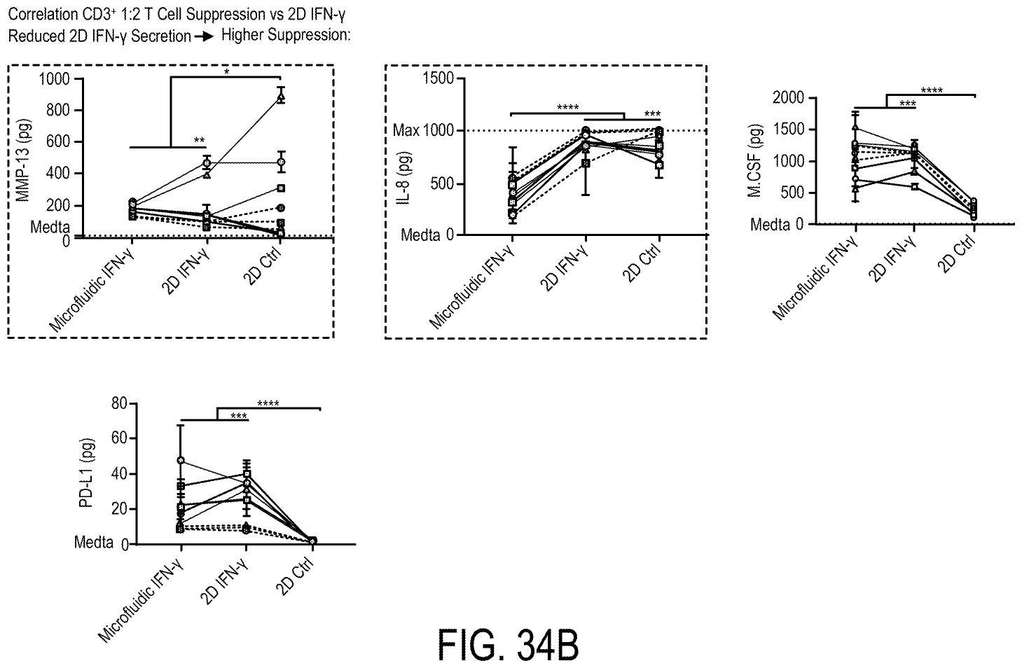

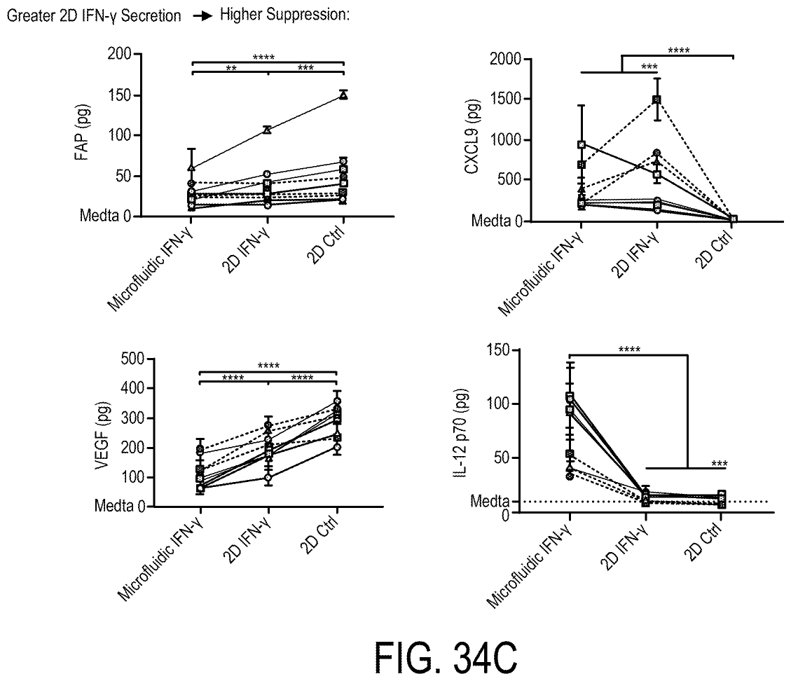

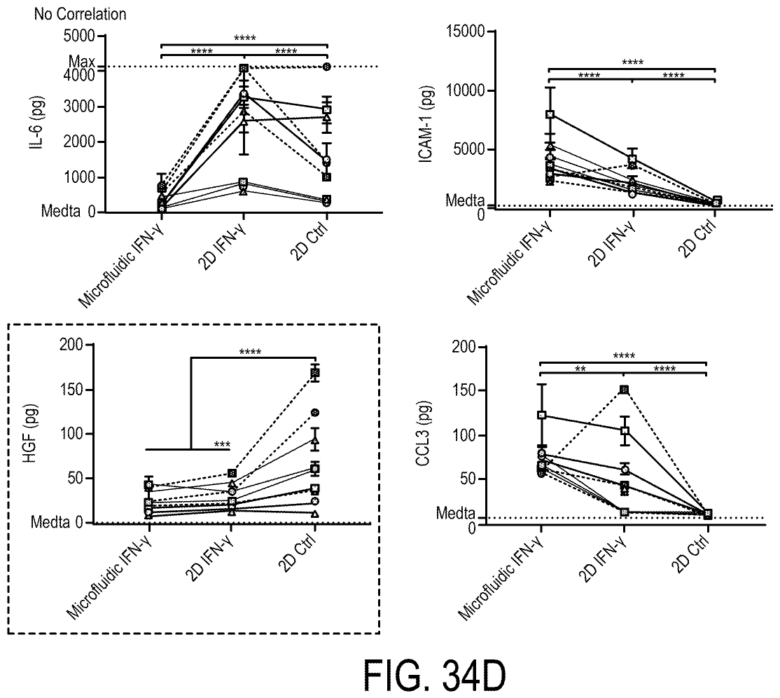

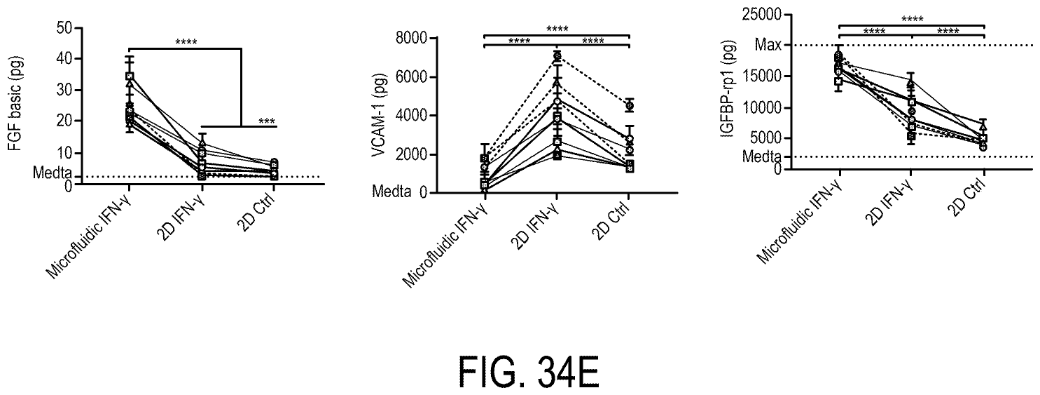

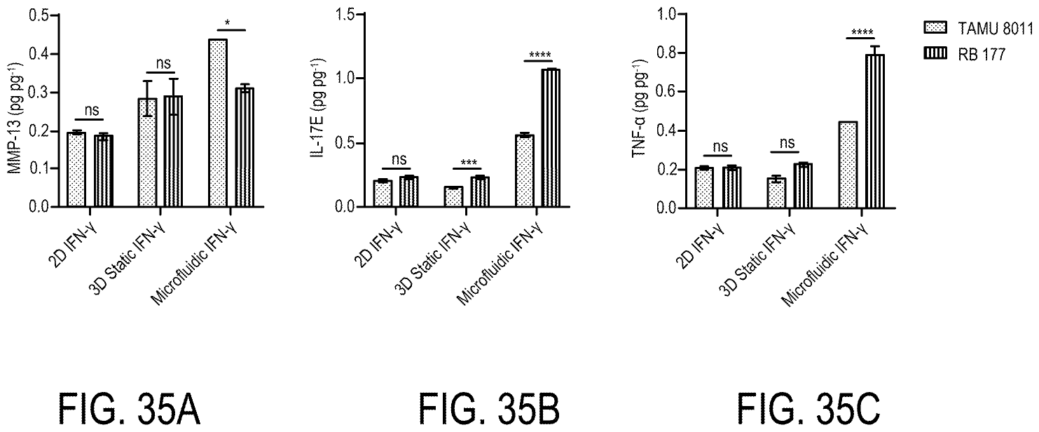

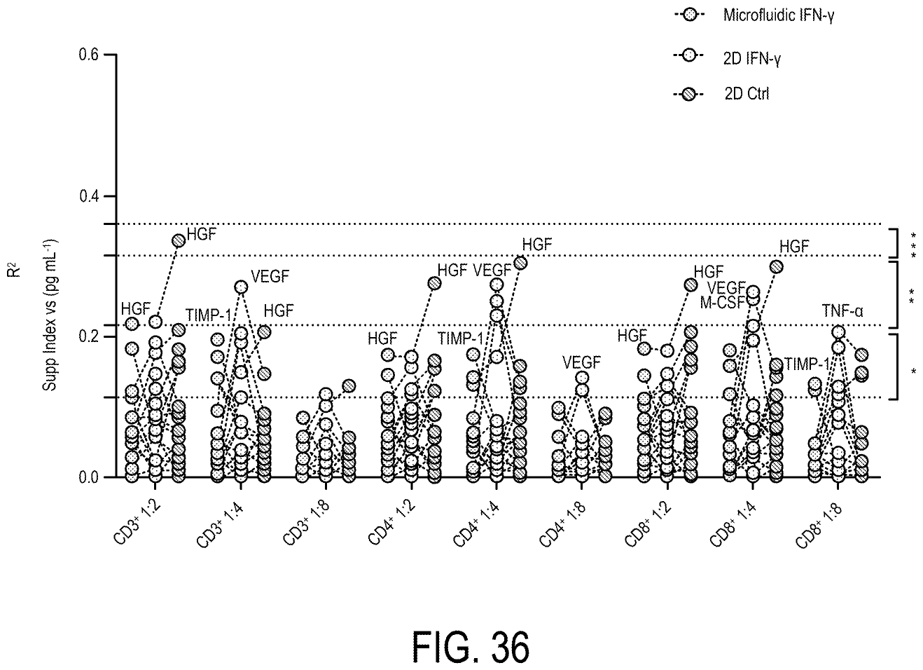

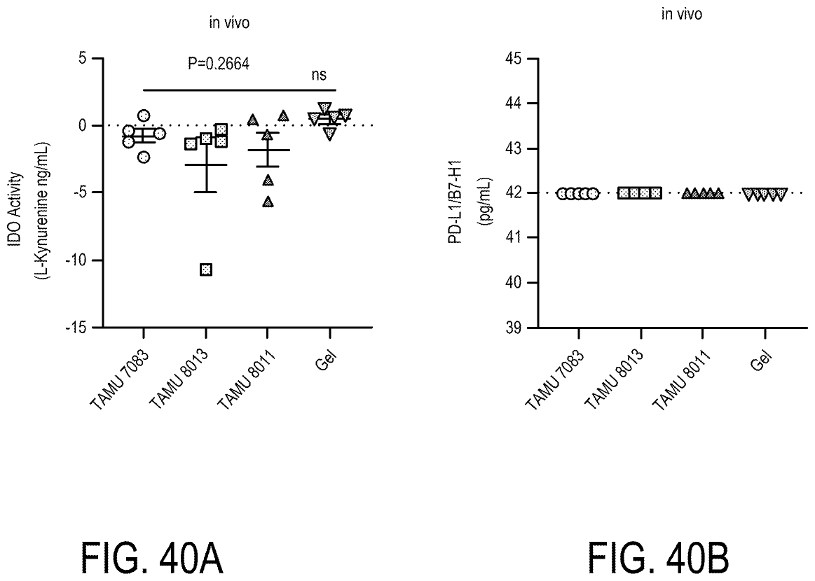

Apart from MMP-13, no other analyte maintained functional potency correlation for both microfluidic IFN-γ and 2D IFN-γ culture systems (FIG. 29, Table 4). Across all functional potency metrics, the overall highest R² observed was CD3⁺ 1:2 suppression index and MMP-13 secretion in microfluidic IFN-γ culture system, and further ranking of this suppression index identified the next three highest performing analytes from the microfluidic IFN-γ system: IL-17E, TNF-α, TIMP-1; the fifth being MMP-13 from the 2D IFN-γ culture system (FIG. 5c). There was a lack of agreement in analyte potency utility between the two systems indicating important culture-driven effects, and raising caution over conclusions informed from 2D culture alone. In support of this, of the 20 analytes measured, only 5 (25%) had positive linear correlation between donor-matched analyte secretion across microfluidic IFN-γ and 2D IFN-γ culture systems, whereas majority of the analytes had no correlation (FIG. 33). No clear patterns were observed from correlative events between the systems (FIG. 34). The secretory trends of the microfluidic IFN-γ system were not maintained in a 3D static IFN-γ system (FIG. 35), and non-normalized analyte secretion levels had overall little correlative success across the 20-plex analyte panel (FIG. 40). Interestingly, analytes that demonstrated potency utility in the microfluidic IFN-γ system are involved in TNF-α/TNF R1 signaling pathways, identifying this cascade as a potential driver of hMSC functional potency. More generally, this result highlights the drastic secretory differences of the same donor cell line in the two systems and emphasizes the need for well-defined culture conditions in potency assay development.

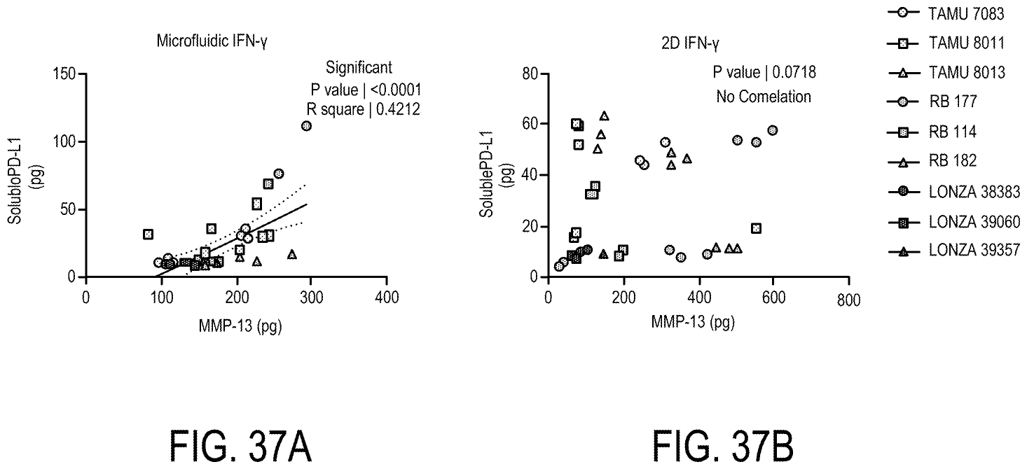

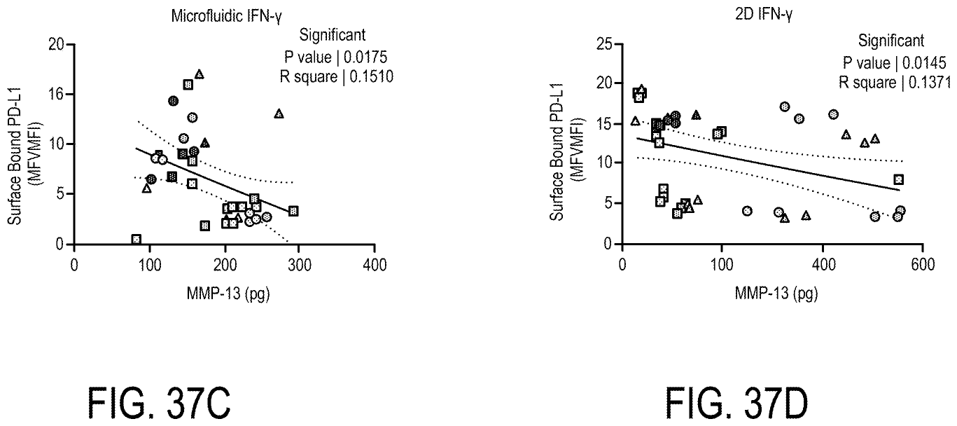

While MMP-13 currently has had limited investigation as an hMSC potency metric, it performs as a direct regulator of PD-L1 presentation and bioavailability (Kasper, 2007) (Dezutter-Dambuyant, 2016). PD-L1 and MMP-13 correlation in the microfluidic IFN-γ culture system uniquely support this anticipated proteolytic interaction (Dezutter-Dambuyant, 2016), and these correlative results further implicate MMP-13 regulation of PD-L1 as a potential controller of hMSC immunomodulatory potential (FIG. 37).

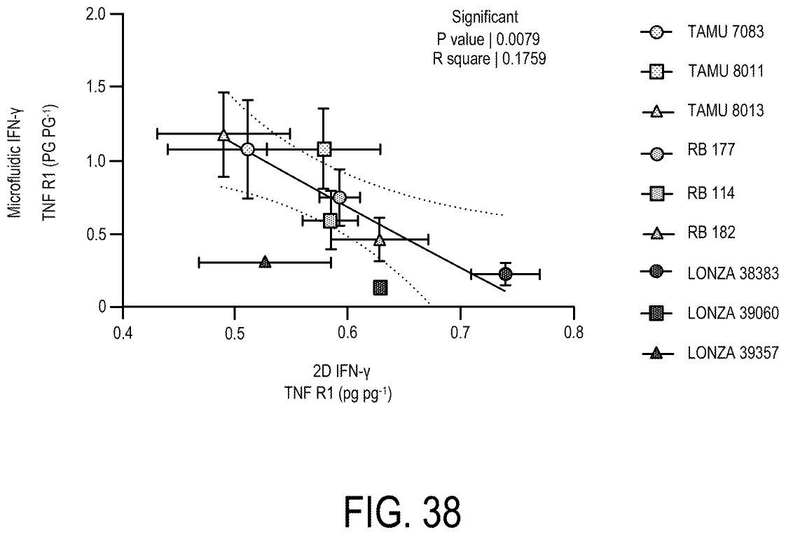

One analyte, TNF R1, a notable hMSC potency marker, showed contradictory potency correlation between microfluidic IFN-γ and 2D IFN-γ culture systems (FIG. 5d) as well as negative correlation between donor-matched microfluidic IFN-γ and 2D IFN-γ secretion levels (FIG. 38). The unsuccessful phase III Prochymal® clinical study (Osiris Therapeutics Inc., now Mesoblast Inc., NCT00366145) used TNF R1 (by ELISA) in 2D culture as a potency assay (Bravery, 2013) (Danilkovitch, 2006) (Kebriaei, 2009). More recently, Mesoblast's BLA submission (BLA 125706, August 2020) was rejected with the FDA citing a lack of demonstrated relevance of the proposed TNF R1 potency assay to in vivo response. In both situations, TNF R1 was suggested as a potency metric where greater secretion indicated greater product potency (FDA, 2020) (Bravery, 2013) (Danilkovitch, 2006) (Kebriaei, 2009) (Mesoblast, Inc., 2020). However, clinical studies of hMSC treatment for graft-versus-host disease reported post-treatment reduction of TNF R1 serum levels for complete response patient cohorts, while observing an increase of serum TNF R1 levels in non-responsive patients (FDA, 2020). The microfluidic IFN-γ system revealed lower TNF R1 secretion correlative of lower suppression index (greater potency) (FIG. 5d); this aligns with clinical reports where reduced levels of TNF R1 were associated with positive patient outcomes (Dander, 2012) (Yin, 2014). On the other hand, the 2D IFN-γ system exhibited lower TNF R1 secretion correlative of greater suppression index (lower potency) (FIG. 5d), consistent with Osiris' and Mesoblast's assay (Danilkovitch, 2006) (Mesoblast, 2020). This contradictory trend was consistent for multiple suppression index T cell subsets and hMSC:T cell ratios (FIG. 5e). The consistent correlation of TNF R1 and potency between clinical serum levels and the microfluidic IFN-γ system demonstrates that the microfluidic system prompts hMSC response with improved clinical relevance compared to conventional 2D IFN-γ culture.

Microfluidic IFN-γ Recapitulates In Vivo hMSC Signaling

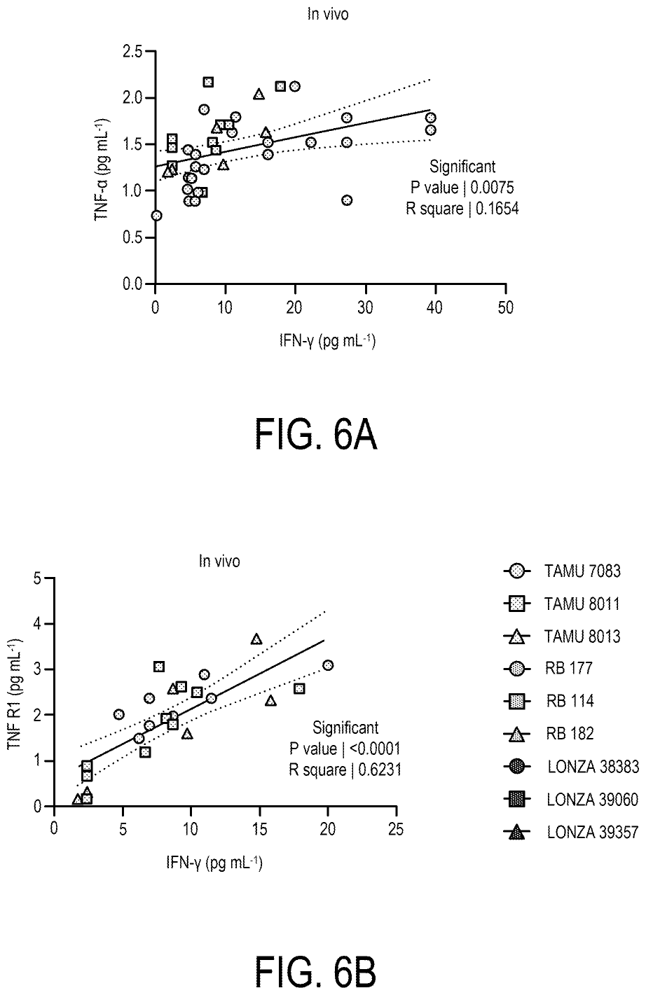

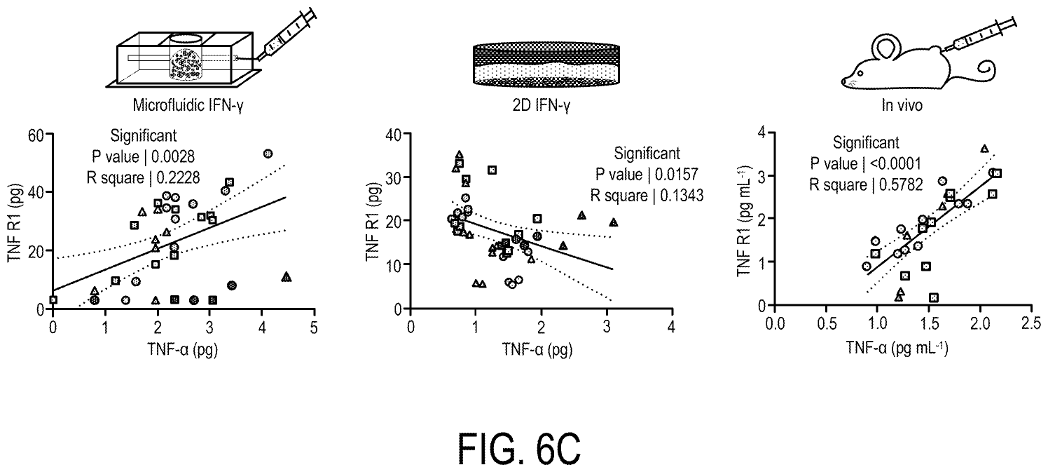

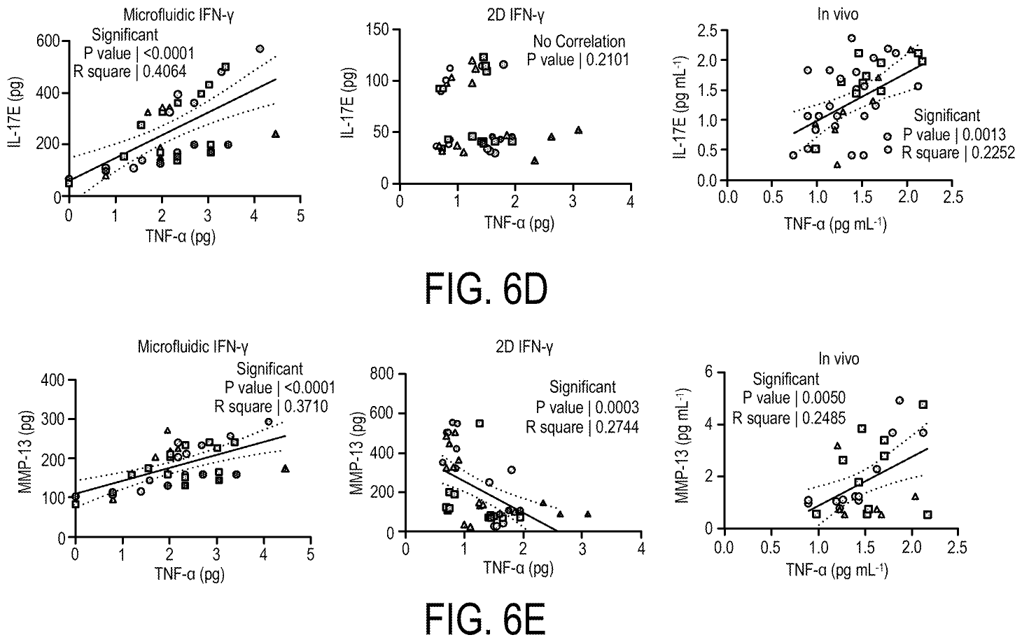

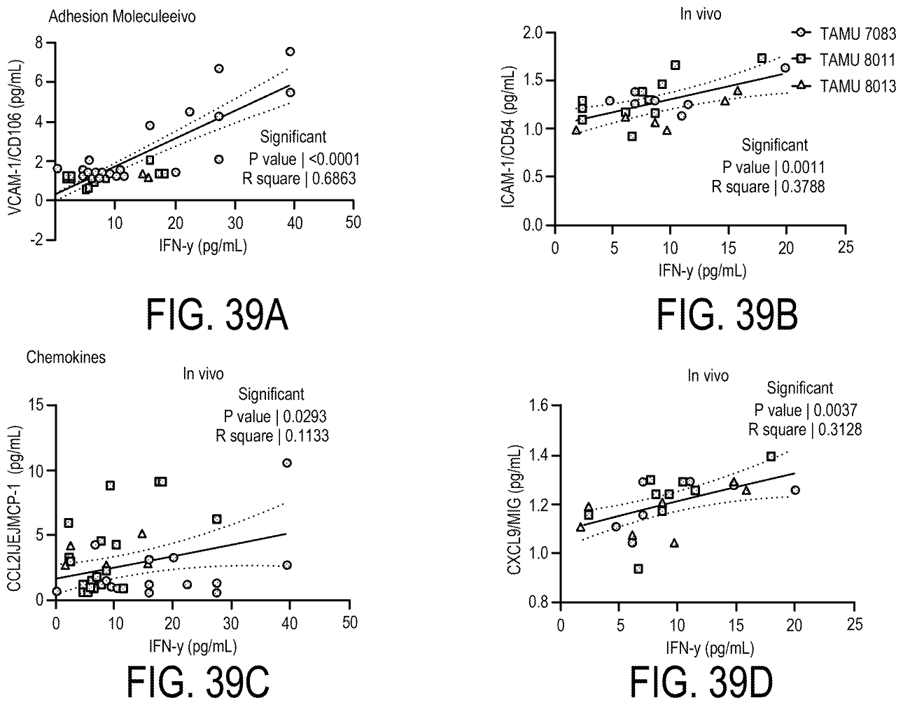

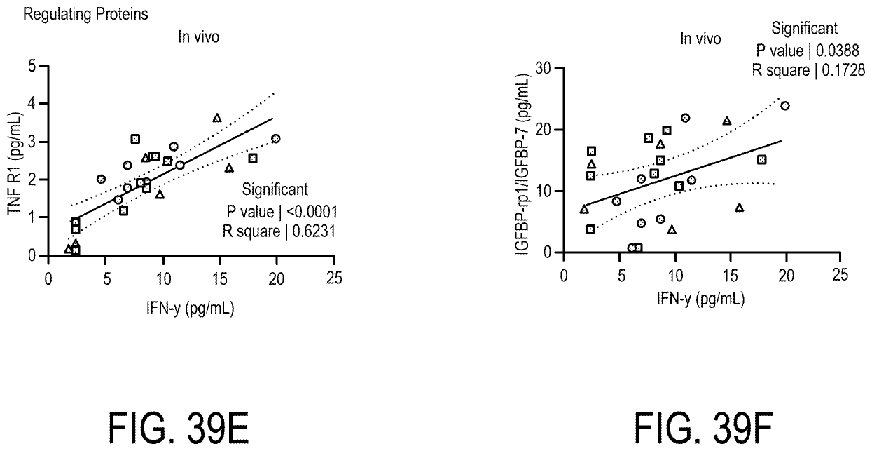

The unique protein signature of hMSCs delivered in vivo was analyzed, specifically focusing on inflammatory signaling of IFN-γ and TNF-α. It is noted that analytes measured from the in vivo model are products of both local secretion and cell lysate. A positive correlation was found between in vivo secreted IFN-γ and TNF-α, consistent with the established positive feedback of these potent inflammatory signaling molecules (FIG. 6a) (Liu, 2011). In vivo IFN-γ levels further correlated to upregulation of adhesion molecules (VCAM-1, ICAM-1), immune cell chemoattractants (CCL2, CXCL9), and other regulators of key metabolic pathways (IGFBP-rp1, TNF R1) (FIG. 39). hMSC expression of adhesion molecules ICAM-1 and VCAM-1 are necessary for T cell immunomodulation (Guangwen, 2010). Interestingly, both IDO activity and soluble PD-L1 were at/below the detection limit of in vivo samples (FIG. 40).

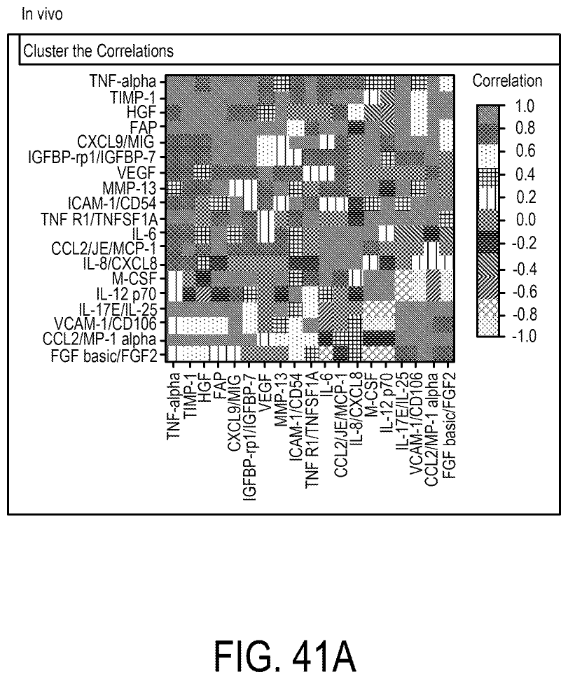

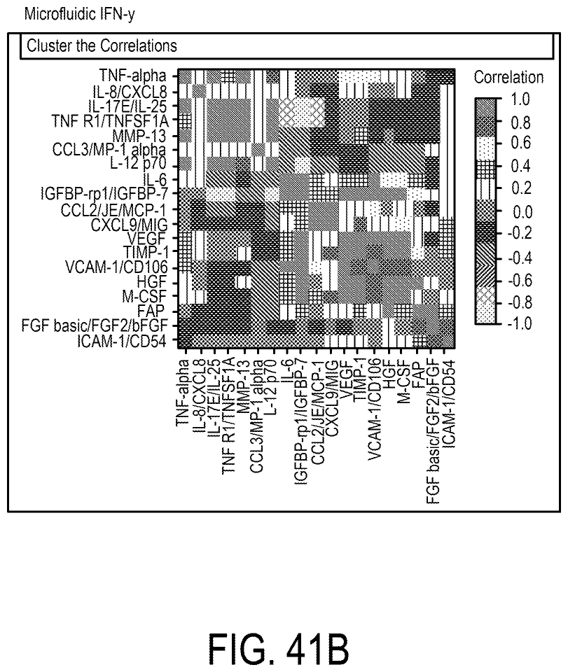

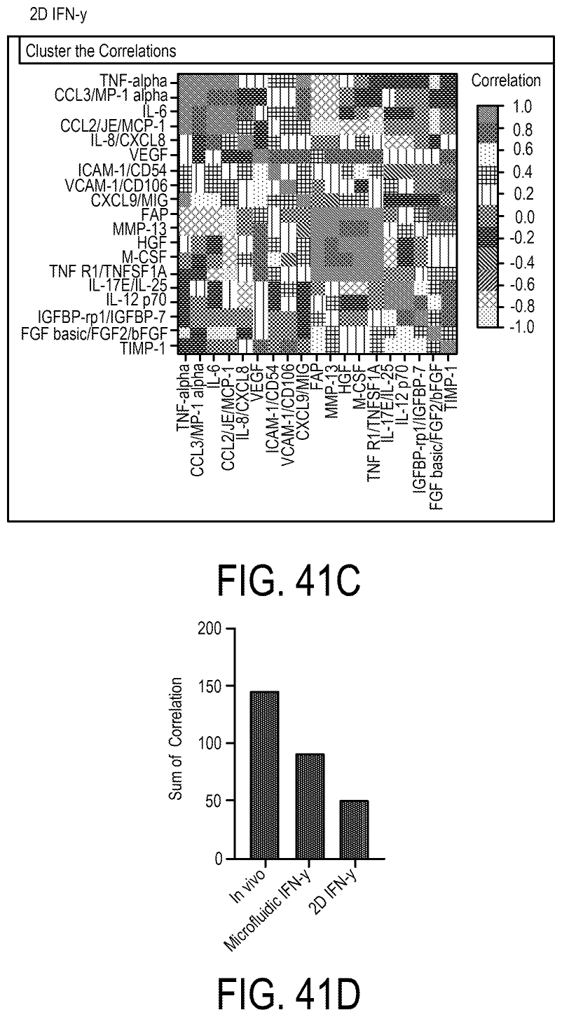

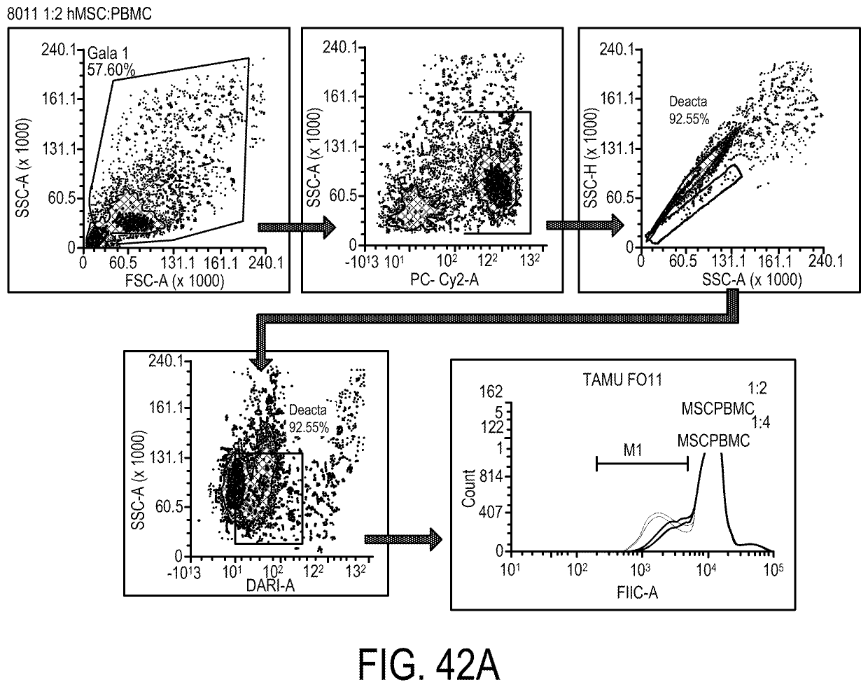

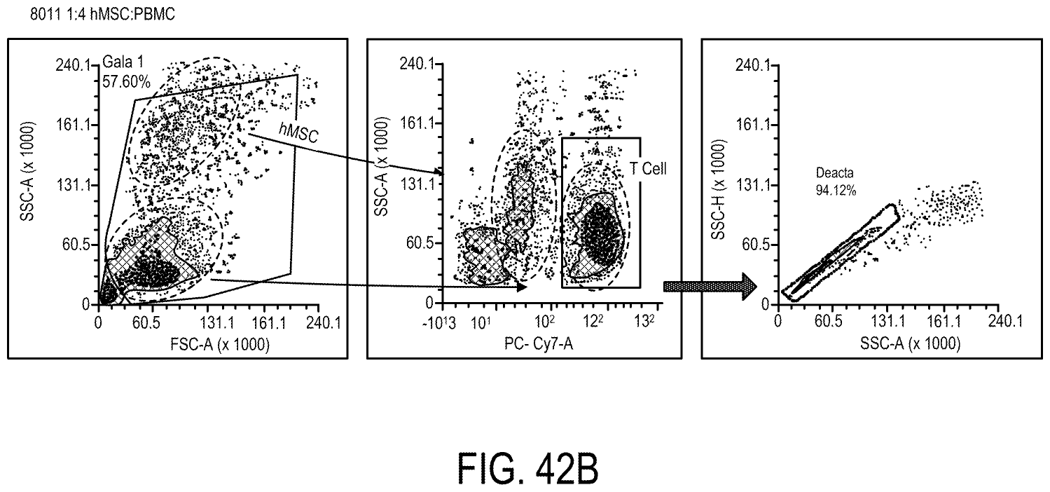

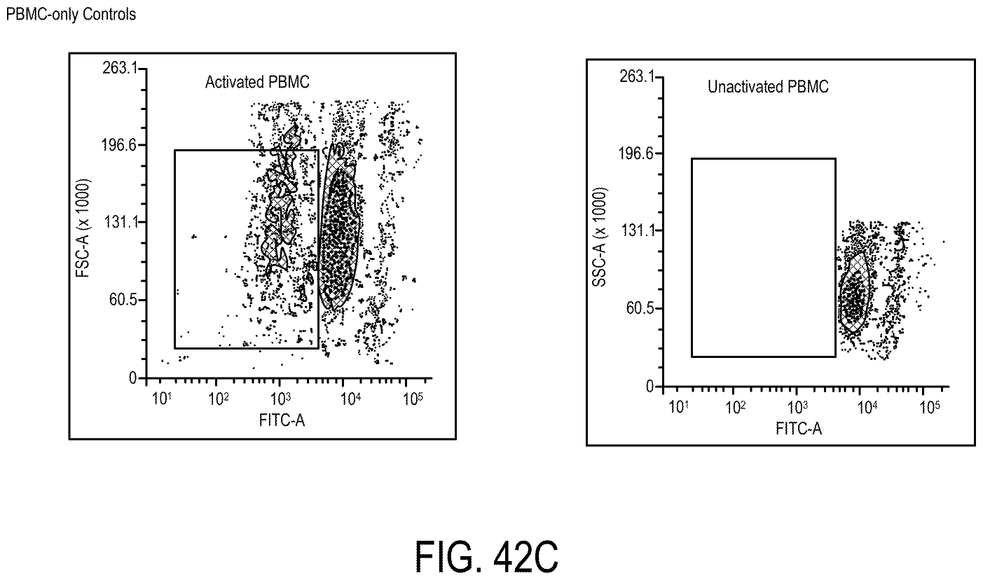

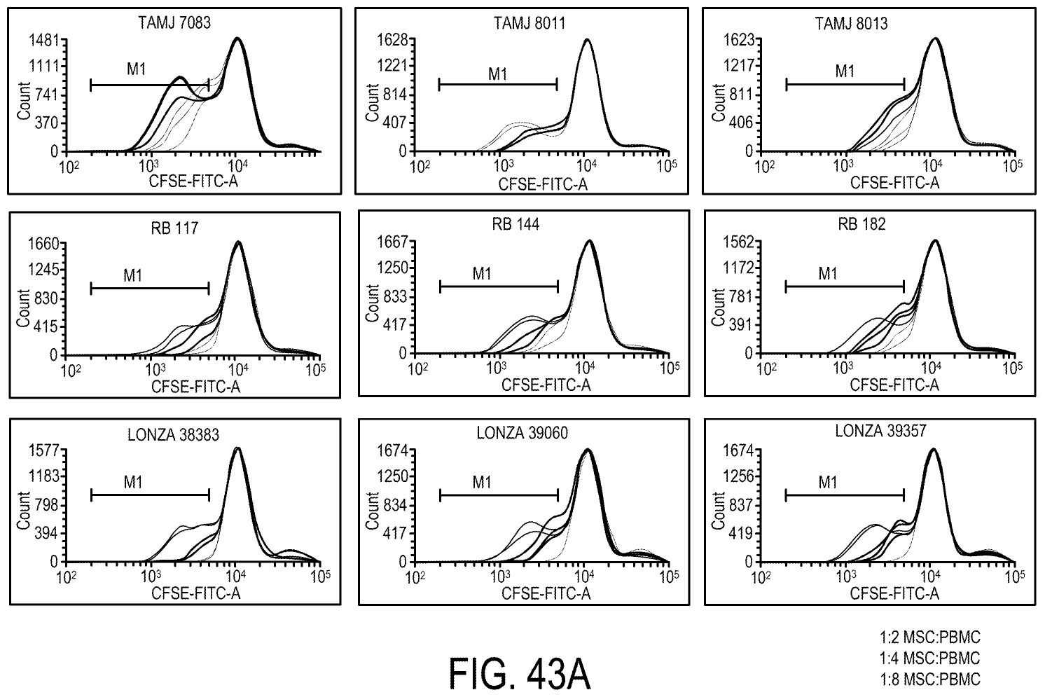

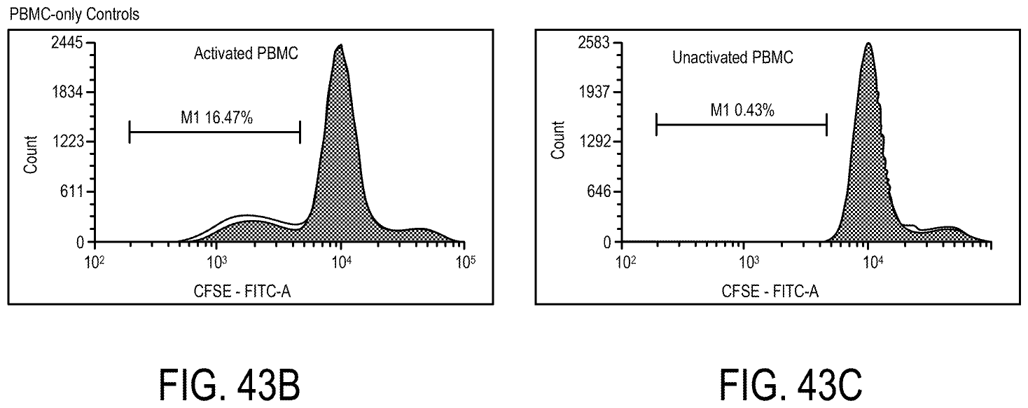

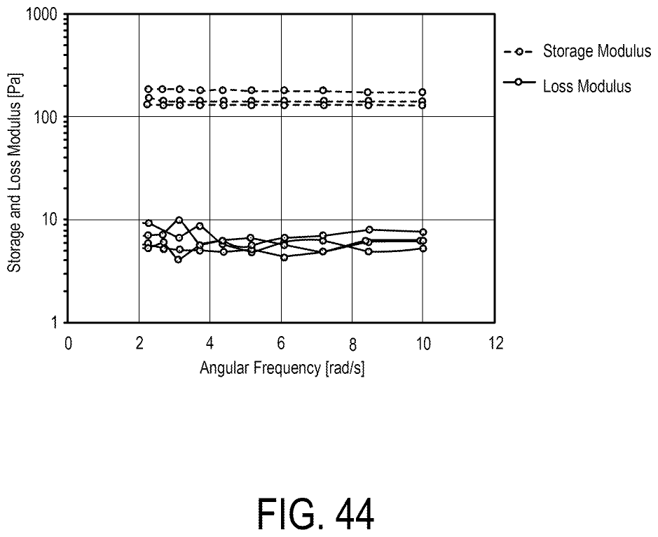

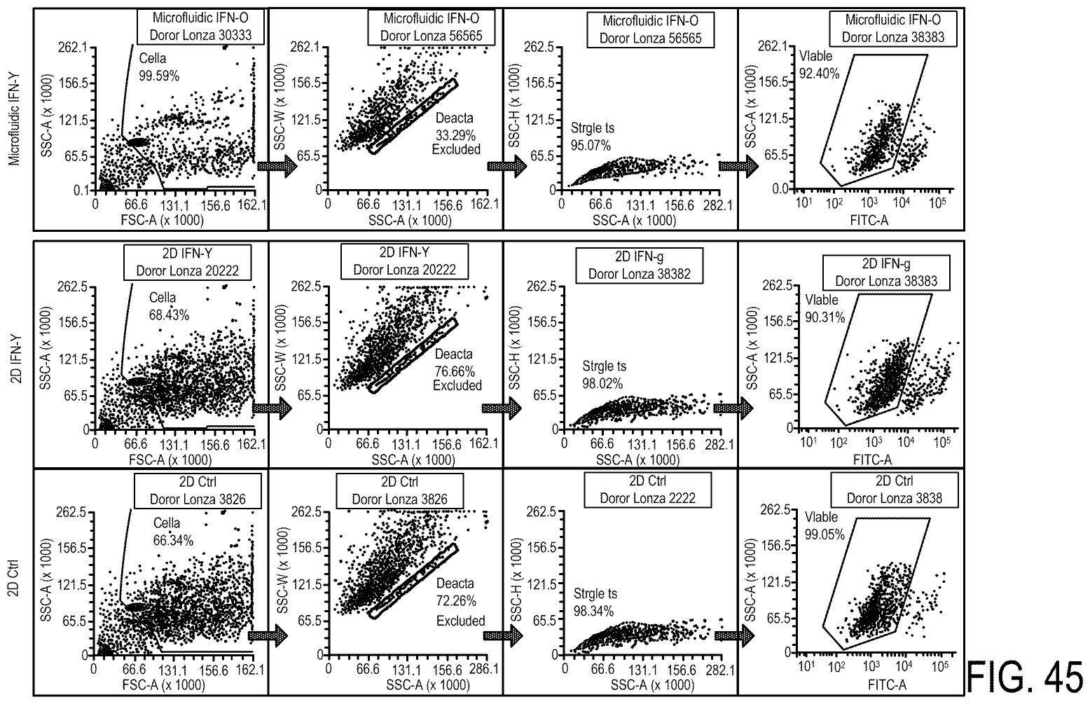

To further evaluate the ability of the microfluidic IFN-γ culture system to recapitulate secretion of hMSCs delivered in vivo, correlative analyte trends and clustering were compared across in vitro and in vivo models. Analysis of correlation clustering demonstrated significant positive correlation among in vivo analytes, where the microfluidic IFN-γ system more closely mimicked these correlative clusters compared to 2D IFN-γ culture (sum of correlation: in vivo 146.0, microfluidic IFN-γ 91.5, 2D IFN-γ 49.5; FIG. 41). To further probe this result, analyte correlations of TNF-α signaling pathway elements were specifically investigated, as motivated by the functional potency prediction observed for both TNF-α and downstream signals in the microfluidic IFN-γ system. TNF-α exists in two potent forms, membrane bound TNF-α and soluble TNF-α; TNF R1 has greater affinity for soluble TNF-α, while TNF R2 is selectively activated by membrane bound TNF-α (Van Hauwermeiren, 2011) (Williams, 2018). Here, the soluble TNF-α/TNR R1 axis was investigated. hMSC-secreted TNF R1 promotes inhibitory T cell responses (immunoregulatory and pro-apoptotic), either by reverse signaling or ligand neutralization (Van Hauwermeiren, 2011) (Martire, 2016). In vivo TNF R1 levels were positively correlated to IFN-γ secretion (FIG. 6b). Notably, there was a positive correlation between TNF-α and TNF R1 for both the in vitro microfluidic IFN-γ and in vivo models. In contrast, these analytes were negatively correlated in the 2D IFN-γ system (FIG. 6c).

Soluble TNF-α/TNF R1 signaling results in two opposing signaling pathways: either pro-inflammatory and anti-apoptotic survival genes or inhibitory pro-apoptotic shift and IL-17 secretion (Van Hauwermeiren, 2011) (Yang, 2018) (Wang, 2014) (Maezawa, 2006). Soluble TNF-α and IL-17E were positively correlated in both the microfluidic IFN-γ system and in vivo model, and no correlation was detected in the 2D IFN-γ system (FIG. 6d). Chronic articular inflammation, a state of high potential for hMSC intervention, is often characterized by tissue TNF-α overabundance and resulting MMP-13 overproduction (Pender, 1998) (Fiedorczyk, 2006). High levels of local MMP-13 contribute to tissue degradation in many inflammatory diseases. A positive correlation was observed between TNF-α and MMP-13 for both the in vitro microfluidic IFN-γ system and in vivo model. In contrast, the 2D IFN-γ culture had a negative correlation (FIG. 6e). Taken together, these results show regulatory agreement of certain TNF-α signaling products in microfluidic IFN-γ culture compared to in vivo environments, with notable disparate secretory responses in 2D IFN-γ culture. This result supports signaling pathways of driving inflammatory mechanisms are better conserved across microfluidic IFN-γ and in vivo systems, but not 2D IFN-γ, indicating improved in vivo secretory recapitulation these signals in the microfluidic IFN-γ system.

### Discussion

Disclosed herein is an on-chip, high-throughput, low-cost microfluidic system as a scalable and robust hMSC potency assay with improved functional predictive power compared to traditional 2D culture. The shortcomings of traditional 2D IFN-γ culture systems for potency assessment are shown, and the microfluidic IFN-γ system as an improved alternative that better mimics physiological hMSC responses is demonstrated. The microfluidic IFN-γ system recapitulates the secretome of in vivo delivered hMSCs with higher fidelity than 2D IFN-γ culture. hMSCs are a highly heterogeneous cell populations that mount potent responses to diverse stimuli. A major driver in the improvement of the microfluidic IFN-γ system is capturing local autocrine and paracrine signaling effects. For the in vivo model and microfluidic IFN-γ system, hMSCs showed strong correlation between inflammatory TNF-α secretion, and counterregulatory signals including anti-inflammatory TNF R1, tissue-degrading MMP-13, and pro-apoptotic IL-17E. This is the first evidence of an in vitro potency assay that supports the ‘medicinal toolbox’ theory behind hMSC treatment (Murphy 2013) the idea that hMSC secretome is locally responsive and personalized to the host in vivo environment. Generally, the results implicate complex auto- and paracrine signaling networks as regulators of hMSC performance, and serve to elucidate hMSC performance.

Several studies have shown that hMSC immunomodulation is regulated by complex immune cell crosstalk, with macrophage/monocyte populations orchestrating initial responses and downstream T cell immunomodulation (Goncalves 2018; Galleu 2017; de Witte 2018). In vitro, hMSCs potently suppress macrophage secreted TNF-α; thus, macrophage-hMSC co-cultures measuring hMSC-mediated TNF-α suppression have been adopted as a functional assay (Pradhan 2020; Robb 2019). Additionally, the role of TNF-α in inflammatory diseases pathology prompted early investigation of TNF R1 as an hMSC surrogate potency marker. The link between analytes in the microfluidic IFN-γ system with correlative power (TNF-α, TNF R1, TIMP-1, MMP-13, IL-17E) and their active role in the TNF-α/TNF R1 signaling pathways supports the role of immune cell crosstalk and implicates macrophages as controllers in hMSC immunomodulation via TNF signaling pathways.

Robust hMSC potency assay development is limited by a lack of relevant clinical outcome correlations and remains a major hurdle of hMSC therapeutic translation. Potency was evaluated using a hMSC:T cell suppression co-culture assay, a measure of hMSC immunomodulatory potential. Prior to this invention, no in vitro assay has been validated for clinical prediction. This additionally holds true for hMSC in vivo models (Galipeau 2018). Thus, disclosed herein is correlative signaling comparisons following hMSC in vivo exposure, rather than a disease outcome model, to support the improved physiological relevance of the system. On-chip assay clinical validation by evaluation of the on-chip system for prediction of patient outcomes is contemplated.

### Experimental Methods

hMSC cell culture: hMSC cell lines were acquired from the NIH Resource Center at Texas A&M University (TAMU; College Station, TX), Roosterbio Inc. (RB; Frederick, MD), or LONZA (provided by Osiris Therapeutics Inc, under U.S. Pat. No. 5,486,359 and others; Basel, CH). All hMSC lines were obtained from healthy willing participants via bone marrow aspirate under IRB-approved protocols and isolated by plastic adherence. Each cell product was certified as hMSCs in accordance with ISCT standards (Galipeau 2016) by surface marker and differentiation characterization by manufacturer. Flow cytometry analyses confirmed cells positive (>90%) for CD166, CD105, CD90, and CD73 and negative (<10%) for CD14, CD34, CD45. TAMU cell lines were cultured with specified media of αMEM (ThermoFisher) with 16.5% MSC qualified FBS (ThermoFisher), 2-4 mM L-glutamine, 100 U mL⁻¹ penicillin (ThermoFisher), 100 pg mL⁻¹ streptomycin (ThermoFisher). RoosterBio cell lines were cultured with RoosterNourish-MSC (KT-001, RoosterBio Inc.). LONZA cell lines were cultured with MSCGM BulletKit (PT-3001, LONZA). All cells were received frozen at P1 or P2. Each vial was expanded and cryopreserved prior to confluence (<80%; P2 or P3). Cells were cultured on sterile tissue culture dishes and lifted with 0.25% trypsin. Each thawed product underwent a 48 hour culture rescue (at P3 or P4) prior to experimentation. Population doubling level (PDL) was calculated:

PDL=3.32*log(Final cell count/initial cell count)+PDL₀

where PDL₀ represents the PDL from most recent harvest. PDL should be reported as cumulative metric beginning at cell isolation (P0); however, TAMU and LONZA could not provide PDL of cell products, thus, PDL calculations began with cell aliquots.

T cell suppression assay: PBMC (Zen-Bio Inc., Donor 1: PBMC-052219A, Donor 2: PBMC-102219B) were washed with anti-aggregate (Fisher Scientific) and then culture rescued for 24 hr in media containing RPMI 1640 HEPES (ThermoFisher), 9% MSC-qualified FBS (ThermoFisher), 100 U mL⁻¹ penicillin (ThermoFisher), 100 pg mL⁻¹ streptomycin (ThermoFisher). On Day 1 of the assay, PBMC were stained with carboxyfluorescein succinimidyl ester (CFSE, ThermoFisher) for generational tracking (Quah 2007). PBMC were counted and from % CD3⁺ T cell provided by manufacturer, approximately 100,000 T cells were seeded per well in a 96-well plate. hMSCs were lifted (0.25% trypsin) and seeded at corresponding ratios to T cells: 1 to 2, 1 to 4, and 1 to 8. Additional wells were maintained for activated and non-activated PBMC-only controls, fluorescent minus one (FMO) controls, and unstained controls. Activation was achieved with anti-CD3⁺/anti-CD28⁺ Dynabeads (ThermoFisher) at a 1:1 T cell to bead ratio. All groups received 12 U rIL-2 (PeproTech). Following 3 days of co-culture, cells were harvested and stained using Zombie UV Fixable Viability Kit, PE-Cy7 anti-human CD3 (UCHT1), PE anti-human CD4 (RPA-T4), APC anti-human CD8 (RPA-T8) all purchased from Biolegend. Gating scheme selected for CD3⁺ T cell population with FMO controls used to set population gates (FIG. 30, 31). Unless otherwise specified, data represents one PBMC donor (Donor 1) across three independent experiments.

Synthesis and set up of on-chip microfluidic system: Microfluidic chips (2.3 cm×2.3 cm; well d=5 mm) of polydimethylsiloxane (PDMS, Sylgard 184 Dow Chemical) were cast in an aluminum macro-mold with steel wire (d=0.02 in) and bonded with oxygen plasma to glass slides. The steel wires were removed to form the fluidic channel. The chip and a thin top PDMS layer were treated by oxygen plasma, the cross-linked cell-laden hydrogel was placed in the PDMS device, and the system was sealed. Fluidic tubing was primed and then connected to provide pressure-driven perfusion of inflammatory recombinant human IFN-γ (Biolegend) supplemented media. Hydrogel synthesis occurred by 20 kDa 4-arm maleimide-functionalized poly(ethylene-glycol) (PEG-4MAL, Laysan Bio) dissolved in 1×PBS (−/−) at 6.8 mM. Adhesive peptide RGD (GRGDSPC, SEQ ID NO: 2, Genscript) was dissolved in 25 mM HEPES buffer at 5 mM. Crosslinkers VPM (GCRDVPMSMRGGDRCG, SEQ ID NO: 1, Genscript) and dithiothreitol (DTT, Sigma-Aldrich) were dissolved in 25 mM HEPES buffer at 24.8 mM and mixed 80:20 ratio by vol. Components were sterilized using 0.22 μm pore filter centrifuge tubes (Costar®, Spin-X®). PEG-4MAL and RGD components were mixed 2:1 vol ratio and allowed to functionalize at room temperature (RT) for 30 minutes. Following 48 hour culture rescue, each donor cell line was harvested, counted (≥95% viability), and suspended in solution at 5×10⁶ cells mL⁻¹. Cells were added to PEG-4MAL+RGD solution just prior to encapsulation at 3:1 vol ratio. Hydrogel gelation occurred by mixing PEG-4MAL+RGD+cell solution and crosslinking solution at 4:1 vol ratio for a 20 μL hydrogel (pre-swelling, 20,000 cells per gel) onto a sterilized hydrophobic surface. Rheological testing of the hydrogel component revealed a loss modulus G″=5.81±0.47 Pa and a storage modulus G′=145±11 Pa (FIG. 32). Microfluidic set up occurred by microfluidic chips attached to tubing (PTFE #30, Cole Parmer) and fed through rubber stoppers of inverted Erlenmeyer flasks with vents (3 devices per flask). Flasks contained αMEM-based culture media with 50 ng mL⁻¹ IFN-γ. The outlets of the microfluidic chips were connected to syringes secured in PHD Ultra Pumps with attached 6/10 multi-racks (Harvard Apparatus) with PTFE tubing (#30 AWG, Cole Parmer), PE/PVC tubing adaptors (0.024×0.064 in, Instech Labs) and 20-gauge blunt tip needles (Industrial Dispensing Tips). Pumps were set to withdraw at 1.0 μL min⁻¹ for 3 days. Following 3 days of culture, syringes were replaced. On day 4, media effluent was collected for multiplex Luminex analysis, and cells were collected for flow cytometry analysis. Experiments were repeated, first with n=6 donors (TAMU, RB; n=4 biological replicates), and subsequently with n=9 donors (TAMU, RB, LONZA; n=3 biological replicates). Samples that clogged (no collection), had less than 25% viability, or less than 100 viable events collected by flow cytometry were excluded from analysis. Unless otherwise specified, analysis represents n=38 microfluidic IFN-γ independent samples.

2D culture system: 2D cultures were run in tandem to microfluidic cultures. Suspended cell solutions discussed prior (5×10⁶ cells mL⁻¹, ≥95% viability) were used for both microfluidic and 2D culture seeding. Cells were seeded onto a tissue culture treated 96-well plate (Costar®) for an initial cell count of 20,000 cells per well. Cells were either treated with control media (2D Ctrl) or media supplemented with 50 ng mL⁻¹ IFN-γ (2D IFN-γ). Following 3 days of culture, supernatant media was removed and replaced with respective control or IFN-γ stimulated media. On day 4, supernatant was collected for multiplex Luminex analysis. Cells were isolated and analyzed by flow cytometry. Each cell donor was run in triplet or quadruplet for each condition. Experiments were repeated, first with n=6 donors (TAMU, RB), and subsequently with n=9 donors (TAMU, RB, LONZA). Unless otherwise specified, analysis represents n=44 2D IFN-γ and n=45 2D Ctrl independent samples.

Flow cytometry: For cell isolation from microfluidic system, the hydrogels were removed and placed in solution of collagenase I (0.2-0.5%), bovine serum albumin (0.1-0.2%), CaCl₂) (10 mM) dissolved in DI water for 30 minutes of incubation or until complete degradation with light agitation. For flow cytometry, viability staining was performed using Zombie Viability. Human Fc-block (TruStain FcX, Biolegend) was added, followed by surface marker staining of APC anti-human B7-H1/PD-L1 (Biolegend, 29E.2A3). Fixation and permeabilization were performed using TrueNuclear Fix/Perm Set (Biolegend), followed by intracellular PE anti-human IDO (ThermoFisher Invitrogen, eyedio). Precision count beads (Biolegend) were added for cell counting. Control groups included FMOs, isotypes, and unstained controls. Gating scheme for one unique experiment is provided (FIG. 33). Data was collected using BD LSR Fortessa Flow cytometer (Becton Dickinson; Franklin Lakes, NJ) high through-put (HTS) collection mode and analyzed using FSC Express v7 (De Novo Software; Glendale, CA). As indicated, results show data compiled (normalized 2D Ctrl, MFI MFI-1; % positive) from two independent experiments with first n=6 donors (TAMU, RB) and subsequently with n=9 donors (TAMU, RB, LONZA).

Imaging flow cytometry: Samples were fixed and stained as described above. Samples (20×10⁶ cells mL⁻¹ in 2% FBS in PBS) were collected using the Amnis MKII Imaging Cytometer (Luminex Corp; Austin, TX) at 40× magnification and analyzed using IDEAS® Analysis Software (Amnis; Seattle, WA).

IDO activity assay: Microfluidic media effluent and 2D media supernatant were collected. Serial dilutions of L-kynurenine (Sigma-Aldrich K8625) were used for generation of standard curves. Briefly, protein precipitate was removed by treating 2:1 with 30% TCA solution (Sigma-Aldrich T6399) in diH₂O. Samples were spun (950 g, 5 min) to achieve precipitate pellet. Without disturbing the pellet, the supernatant was collected for further reaction steps. Ehrlich's reagent solution was prepared by diluting 100 mg Ehrlich's reagent (4-(dimethylamino)benzaldehyde, Sigma-Aldrich, 156477) in 5 mL glacial acetic acid (Sigma-Aldrich, ARK2183). Samples were reacted with Ehrlich's reagent solution at 1:1 and incubated for 5 minutes at 37° C. Sample absorbance was read at 490 nm with plate reader (BioTek; Winooski, VT). Background absorbance (media) was subtracted from each standard and sample absorbance measurement. The standard curve was used to quantify L-kynurenine concentration (ng mL⁻¹) in samples. Where necessary, samples below the detection limit (0.02 ng mL⁻¹) were set at this minimum level.

Multiplex Luminex: Custom made Luminex 20-plex panels were purchased from R&D Biotech. Analytes were screened using a variety of custom kits (R&D) and Human XL Discovery Kit (R&D, LKTM014), and 20 were chosen for analysis. PD-L1 was at/below the detection limit for in vivo samples, and was excluded from multivariate analysis that included in vivo multiplex Luminex data. The Microparticle Cocktail was incubated with samples at 4° C. overnight with light agitation for improved assay sensitivity. Kit analyte standards were run in tandem. Sample washing was performed using automated 405 LS Washer (BioTek; Winooski, VT) and analyzed with MAGPIX® System (Luminex Corp; Austin, TX). Analyte concentrations (pg mL⁻¹) were calculated from best fit curves using Milliplex Analyst Software (Luminex Corp; Austin, TX). Individual samples above/below the analyte detection limit were taken at the maximum/minimum detection for further analysis. Analytes with majority of samples out of range were excluded from analysis.

In vivo studies: All animal experiments were performed with approval of Georgia Tech Animal Care and Use Committee under GARCIA-A100318 with veterinary supervision and within the guidelines of the Guide for the Care and Use of Laboratory Animals. Immunocompromised NSG mice (Jackson Laboratory; Bar Harbor, ME) were anesthetized under isoflurane and dorsal hair was removed for hydrogel delivery. Hydrogel solution with cells (50 μL with 200,000 cells) was injected into the subcutaneous dorsal region of the mouse. Four samples were injected per mouse, where each mouse received one of the four groups (randomized) in each dorsal quadrant. Where hMSC donors were compared, in vivo studies were blinded. Hydrogels were retrieved for secretory analysis at 3 days following injection. Tissue extraction buffer was made fresh with 100 mM Tris pH 7.4, 150 mM NaCl, 1 mM EDTA, 1% Triton X-100, 0.5% sodium deoxycholate, protease and phosphatase inhibitors (leupeptin, pepstatin A). Following euthanasia, an incision was made the length of the mouse spine, followed by perpendicular incisions, to expose hydrogel samples. A biopsy punch (10 mm) was made around each hydrogel sample, excising the complete hydrogel and local skin tissue. In groups without hydrogels (cell solution, saline) the injection point was marked, and the same processes were followed. Samples were weighed and placed in buffer at 250 mg mL⁻¹ and agitated gently for 60 minutes on ice. Each sample was further homogenized by sonication in 5 second intervals at 70% amplitude for a minimum of 5 repetitions. Sonication was repeated until samples were well digested. Samples were centrifuged at 14,000×g for 30 minutes at 4° C. prior to performing the Luminex assay. Total protein was measured from using Pierce™ Rapid Gold BCA Protein Assay Kit (ThermoFisher, A53227). Samples without intact hydrogels at explanation or with low beads counts for Luminex analysis were excluded. Three independent in vivo studies were performed (n=6, n=4, n=7; with TAMU 8011, TAMU 8013, and TAMU 7083).

Linear regression analysis: Data was analyzed using GraphPad Prism 8 (GraphPad Software Inc., La Jolla, CA). Linear regression analysis was performed where indicated with R² and corresponding P values shown. X values were input as means while Y replicates were treated as independent samples. Best fit lines are represented as solid lines and corresponding 95% confidence bands with dotted lines. P values represent confidence against a null hypothesis of slope deviation from zero. P<0.05 is significant. Where indicated, *P<0.0332, **P<0.0021, ***P<0.0002, ****P<0.0001 as to best approximation with P values provided (Table 1, 2, and 4). All data represented as means±SEM.

Clustering and multivariate analysis: Hierarchal clustering, multivariate discriminant analysis, and correlation clusters were performed using JMP Pro 15 (IMP Software from SAS; Cary, NC). Hierarchal clustering utilized Ward Method clustering method, with data standardized by analyte. Multivariate discriminant analysis used linear discriminant method with inner group ellipse representing 95% confidence that region contains true mean and outer ellipse representing estimated region to contain 50% of population.

Statistical analysis: Tests were analyzed by GraphPad Prism 8 (GraphPad Software Inc., La Jolla, CA). Statistical tests are specified and P values provided. For in vivo studies, unpaired one-way ANOVA tests are used with multiple comparison between means of each group. For in vitro assays with single comparison, two-tailed unpaired t test with Welch's correction was used. For in vitro assays with multiple comparison, two-way ANOVA tests were performed across group means of n=9 donors with indicated mean comparisons. *P<0.0332, **P<0.0021, ***P<0.0002, ****P<0.0001. All replicates indicated were performed as discrete samples or biological replicates. All data represented as means SEM.

### Additional Discussion

### PD-L1 as Potency Metric in 2D Culture

The potency utility of surface bound and soluble PD-L1 (also known as B7-H1), another putative hMSC potency marker, was assessed (Levy 2020; Chinnadurai 2014; Davies 2016). PD-L1 binding to PD-1 receptors is a well-established pathway of hMSC-mediated T cell suppression by cell-contact (Chinnadurai 2014) and secretory (Davies 2016) signaling. The impact of hMSC-produced soluble PD-L1 is likely multifaceted as various mechanisms of PD-L1 release have been identified (Dezutter-Dambuyant 2016; Cha 2019; Chen 2018) and soluble ligand activity is likely dependent on the mechanism of release. Similarly to IDO, different measurements of PD-L1 are often equated to hMSC potency, without a clear distinction of the specific form of product measured. To investigate hMSC PD-L1 potency utility, surface bound PD-L1 was measured by flow cytometry and soluble PD-L1 in the cell supernatant by Luminex assay. The surface bound localization of PD-L1 flow cytometry was confirmed with imaging flow cytometry (FIG. 2b, FIG. 10). Both surface bound and soluble levels of PD-L1 were upregulated upon IFN-γ stimulation for all hMSC donors, and surface bound PD-L1 levels were negatively correlated to soluble PD-L1 levels (FIG. S7). Linear regression analysis with T cell suppression indices found no potency utility of surface bound PD-L1, and a positive correlation with soluble PD-L1 (FIG. S7). Linear regression analysis was repeated for other surface expression PD-L1 metrics (Table 2). Generally, the results show a negative correlation between surface bound and soluble PD-L1 and inconsistent PD-L1 potency metric indications, suggesting regulatory mechanisms as controllers of PD-L1 presentation with impact on immunomodulatory potential. Taken together, these findings reveal limitations of PD-L1 as a potency marker.

### Evaluation of PD-L1 as Potency Metric in Microfluidic IFN-γ System

Potency analyses of surrogate potency marker PD-L1 was performed for hMSC in microfluidic IFN-γ culture system. Consistent with 2D IFN-γ, microfluidic IFN-γ had a negative correlation between surface bound PD-L1 expression and PD-L1 secretion (FIG. 28). A positive correlation was detected between suppression index and soluble PD-L1 in 2D IFN-γ culture and a negative correlation of suppression index and surface bound PD-L1 in microfluidic IFN-γ culture (FIGS. 13, 28). The more potent donors were most effective at repressing PD-L1 secretion in 2D IFN-γ culture and upregulating surface bound PD-L1 in microfluidic IFN-γ culture. Taken in conjunction with the negative correlation between PD-L1 products, the results support the hypothesized mechanism of PD-L1 cleavage to limit hMSC immunosuppressive potential (Dezutter-Dambuyant 2016). This analysis conducted for other metrics of surface bound PD-L1 expression (% PD-L1 positive) (Guan 2018) lacked correlation for suppression index (Table 2), further underscoring the limitations of PD-L1 as a potency metric.

PD-L1-MMP-13 Interactions and Role in hMSC Immunomodulatory Potential

MMP-13 plays a central role in protein regulation via proteolytic cleavage controlling protein presentation and bioavailability (Kasper 2007). Importantly, PD-L1, an immunoregulatory signaling ligand, is specifically cleaved by MMP-13 (Dezutter-Dambuyant 2016). As supported in the analysis, this enzymatic cleavage to soluble PD-L1 corresponds to reduced immunosuppressive potential Dezutter-Dambuyant 2016). A strong correlation was identified between T cell suppression and MMP-13 levels, where a more immunosuppressive hMSC product exhibited lower MMP-13 levels (FIG. 28). Further analysis revealed strong positive linear correlation of the microfluidic IFN-γ system between MMP-13 and soluble PD-L1 and the inverse relationship for surface bound PD-L1 (FIG. 37). The most potent LONZA donors maintained low MMP-13, low soluble PD-L1, and higher surface-bound PD-L1. This result shows that MMP-13-PD-L1 interaction influences hMSC immunomodulatory potential with this mediation uniquely captured in the microfluidic IFN-γ system.

### Tables

TABLE 1
P values of IDO metrics for 2D IFN-γ and microfluidic IFN-γ culture
systems. Raw MFI linear regression was performed using data from single experiment.

| T cell subset | | | | | | | | |
|---|---|---|---|---|---|---|---|---|
| CD3⁺ | | | CD4⁺ | | | CD8⁺ | | |
| hMSC:T cell ratio | | | | | | | | |
| 1:2 | 1:4 | 1:8 | 1:2 | 1:4 | 1:8 | 1:2 | 1:4 | 1:8 |

Microfluidic IFN-γ

| | | | | | | | | | |
|---|---|---|---|---|---|---|---|---|---|
| IDO Expression (MFI) | 0.3112 | 0.1565 | 0.3605 | 0.3310 | 0.1359 | 0.0933 | 0.3675 | 0.1403 | 0.3515 |
| IDO Expression Fold Inc (MFI MFI⁻¹) | 0.9632 | 0.7111 | 0.4166 | 0.9327 | 0.6482 | 0.9062 | 0.9159 | 0.5985 | 0.7726 |
| IDO Expression (%) | 0.0302 | 0.0075 | 0.1952 | 0.0414 | 0.0110 | 0.0165 | 0.0569 | 0.0107 | 0.2203 |
| IDO Activity (ng mL⁻¹) | 0.4293 | 0.7756 | 0.3266 | 0.3146 | 0.6816 | 0.2637 | 0.3391 | 0.7631 | 0.7371 |
| IDO Activity per cell (ng cell⁻¹) | 0.0210 | 0.3181 | 0.4087 | 0.0086 | 0.1602 | 0.0813 | 0.0103 | 0.2076 | 0.6799 |

2D IFN-γ

| | | | | | | | | | |
|---|---|---|---|---|---|---|---|---|---|
| IDO Expression (MFI) | 0.5996 | 0.6334 | 0.2446 | 0.3578 | 0.5149 | 0.0685 | 0.4950 | 0.6816 | 0.5227 |
| IDO Expression Fold Inc (MFI MFI⁻¹) | 0.8890 | 0.9203 | 0.7712 | 0.7179 | 0.918 | 0.3941 | 0.8616 | 0.9807 | 0.4017 |
| IDO Expression (%) | 0.1400 | 0.0175 | 0.0896 | 0.1190 | 0.0249 | 0.0086 | 0.2336 | 0.0429 | 0.9688 |
| IDO Activity (ng mL⁻¹) | 0.0053 | 0.0014 | 0.1421 | 0.0136 | 0.0005 | 0.0608 | 0.0142 | 0.0004 | 0.0183 |
| IDO Activity per cell (ng cell⁻¹) | 0.0083 | 0.0045 | 0.5618 | 0.0192 | 0.0016 | 0.1902 | 0.0172 | 0.0008 | 0.1004 |

TABLE 2
P values of PD-L1 metrics for microfluidic IFN-γ and 2D IFN-γ
systems. Raw MFI linear regression was performed using data from single experiment.

| T cell subset | | | | | | | | |
|---|---|---|---|---|---|---|---|---|
| CD3⁺ | | | CD4⁺ | | | CD8⁺ | | |
| hMSC:T cell ratio | | | | | | | | |
| 1:2 | 1:4 | 1:8 | 1:2 | 1:4 | 1:8 | 1:2 | 1:4 | 1:8 |

Microfluidic IFN-γ

| | | | | | | | | | |
|---|---|---|---|---|---|---|---|---|---|
| Surface Bound PD-L1 (MFI) | 0.8859 | 0.4897 | 0.7657 | 0.9495 | 0.6629 | 0.5952 | 0.9533 | 0.5672 | 0.5738 |
| Surface Bound PDL1 Fold Inc (MFI MFI⁻¹) | 0.0202 | 0.2942 | 0.6847 | 0.0248 | 0.0897 | 0.5684 | 0.0184 | 0.0904 | 0.4425 |
| Surface Bound PD−L1 (%) | 0.1658 | 0.7516 | 0.4526 | 0.1121 | 0.3400 | 0.4858 | 0.1476 | 0.3293 | 0.3119 |
| Soluble PD−L1 (pg pg⁻¹) | 0.1432 | 0.4568 | 0.8562 | 0.1128 | 0.1757 | 0.3262 | 0.1044 | 0.1743 | 0.6483 |

2D IFN-γ

| | | | | | | | | | |
|---|---|---|---|---|---|---|---|---|---|
| Surface Bound PD-L1 (MFI) | 0.0145 | 0.0503 | 0.6593 | 0.0469 | 0.0413 | 0.8267 | 0.0321 | 0.0263 | 0.2489 |
| Surface Bound PDL1 Fold Inc (MFI MFI⁻¹) | 0.1223 | 0.2427 | 0.6705 | 0.0987 | 0.1522 | 0.2278 | 0.1505 | 0.2199 | 0.6456 |
| Surface Bound PD-L1 (%) | 0.4693 | 0.2475 | 0.206 | 0.465 | 0.0079 | 0.0945 | 0.0469 | 0.0125 | 0.0818 |
| Soluble PD-L1 (pg pg⁻¹) | 0.0185 | 0.1331 | 0.8799 | 0.0235 | 0.0516 | 0.3552 | 0.0246 | 0.0545 | 0.7087 |

TABLE 3
20-plex analyte panel informed from in vivo
studies and associated protein acronyms.

| Protein Name | Associated acronym(s) |
|---|---|
| Tumour necrosis factor alpha | TNF-α |
| Interleukin 6 | IL-6 |
| Programmed death-ligand 1 | PD-L1, B7-H1 |
| Interleukin 8 | IL-8, CXCL8 |
| Fibroblast activation protein alpha | FAP |
| Chemokine ligand 2 (or monocyte | CCL2, JE, MCP-1 |
| chemoattractant protein 1) | |
| Vascular endothelial growth factor | VEGF |
| Interleukin 17E | IL-17E, IL-25 |
| Matrix metallopeptidase 13 | MMP-13 |
| Hepatocyte growth factor | HGF |
| Chemokine ligand 3 (or macrophage | CCL3, MIP-1 alpha |
| inflammatory protein 1 alpha) | |
| Basic fibroblast growth factor | FGF basic, FGF2, bFGF |
| Macrophage colony-stimulating factor | M-CSF |
| Chemokine ligand 9 (or monokine | CXCL9, MIG |
| induced by gamma interferon) | |
| Interleukin 12 p70 | IL-12 p70 |
| Vascular cell adhesion molecule 1 | VCAM-1, CD106 |
| Intracellular adhesion molecule 1 | ICAM-1, CD54 |
| Tumor necrosis factor receptor 1 | TNF R1, TNFRSF1A |
| TIMP metallopeptidase inhibitor 1 | TIMP-1 |
| Insulin-like growth factor binding | IGFBP-rp1, IGFBP-7 |
| protein-related | |

TABLE 4
P value of linear regression for 20-plex analytes of microfluidic IFN-γ and 2D IFN-γ systems.

| T cell subset | | | | | | | | |
|---|---|---|---|---|---|---|---|---|
| CD3⁺ | | | CD4⁺ | | | CD8⁺ | | |
| hMSC:T cell ratio | | | | | | | | |
| 1:2 | 1:4 | 1:8 | 1:2 | 1:4 | 1:8 | 1:2 | 1:4 | 1:8 |

Microfluidic IFN-γ

| | | | | | | | | | |
|---|---|---|---|---|---|---|---|---|---|
| TNF-alpha | 0.0031 | 0.0509 | 0.6351 | 0.0056 | 0.0394 | 0.2278 | 0.0063 | 0.0377 | 0.3105 |
| IL-6 | 0.1905 | 0.8899 | 0.1376 | 0.2121 | 0.6519 | 0.6075 | 0.1495 | 0.5199 | 0.9161 |
| PD-L1/B7-H1 | 0.1432 | 0.4568 | 0.8562 | 0.1128 | 0.1757 | 0.3262 | 0.1044 | 0.1743 | 0.6483 |
| IL-8/CXCL8 | 0.2144 | 0.3138 | 0.3903 | 0.2225 | 0.1994 | 0.3367 | 0.1888 | 0.1809 | 0.1378 |
| FAP | 0.168 | 0.1542 | 0.1217 | 0.2435 | 0.1492 | 0.2984 | 0.1667 | 0.1005 | 0.0037 |
| CCL2/JE/MCP-1 | 0.0453 | 0.2321 | 0.967 | 0.0745 | 0.2797 | 0.6906 | 0.0402 | 0.1658 | 0.0897 |
| VEGF | 0.4132 | 0.8792 | 0.7866 | 0.3129 | 0.9039 | 0.8429 | 0.3653 | 0.8324 | 0.3842 |
| IL-17E/IL-25 | 0.0006 | 0.009 | 0.3336 | 0.0016 | 0.0076 | 0.1151 | 0.0013 | 0.0056 | 0.0628 |
| MMP-13 | <0.0001 | <0.0001 | 0.0122 | <0.0001 | <0.0001 | 0.0019 | <0.0001 | <0.0001 | <0.0001 |
| HGF | 0.6669 | 0.8742 | 0.0654 | 0.5251 | 0.6503 | 0.2248 | 0.548 | 0.7097 | 0.3628 |
| CCL3/MIP-1 alpha | 0.1377 | 0.8091 | 0.7704 | 0.066 | 0.8266 | 0.582 | 0.0618 | 0.8777 | 0.7677 |
| FGF basic/FGF2/bFGF | 0.8847 | 0.3214 | 0.9525 | 0.7177 | 0.3637 | 0.5828 | 0.6627 | 0.3489 | 0.8804 |
| M-CSF | 0.2865 | 0.3282 | 0.4606 | 0.32 | 0.1632 | 0.3589 | 0.2314 | 0.1281 | 0.0426 |
| CXCL9/MIG | 0.5709 | 0.6824 | 0.9686 | 0.4287 | 0.8323 | 0.7856 | 0.4128 | 0.7923 | 0.8557 |
| IL-12 p70 | 0.3437 | 0.4435 | 0.8152 | 0.3386 | 0.3673 | 0.4722 | 0.3332 | 0.3242 | 0.8074 |
| VCAM-1/CD106 | 0.3177 | 0.5831 | 0.7113 | 0.3636 | 0.5686 | 0.869 | 0.3754 | 0.5584 | 0.8912 |
| ICAM-1/CD54 | 0.0518 | 0.608 | 0.788 | 0.0287 | 0.1682 | 0.393 | 0.031 | 0.2205 | 0.9313 |
| TNFR1/TNFRSF1A | 0.0097 | 0.0671 | 0.6043 | 0.0134 | 0.0181 | 0.141 | 0.0132 | 0.0212 | 0.2182 |
| TIMP-1 | 0.0037 | 0.0059 | 0.0868 | 0.0092 | 0.0081 | 0.0499 | 0.0088 | 0.0071 | 0.0229 |
| IGFBP-rp1/IGFBP-7 | 0.0508 | 0.1272 | 0.4982 | 0.0903 | 0.1243 | 0.4242 | 0.0924 | 0.1445 | 0.3131 |

2D IFN-γ

| | | | | | | | | | |
|---|---|---|---|---|---|---|---|---|---|
| TNF-alpha | 0.7276 | 0.9459 | 0.1354 | 0.6553 | 0.7639 | 0.5779 | 0.9749 | 0.9805 | 0.0003 |
| IL-6 | 0.0843 | 0.9608 | 0.0744 | 0.116 | 0.9991 | 0.1804 | 0.1235 | 0.977 | 0.1667 |
| PD-L1/B7-H1 | 0.0185 | 0.1331 | 0.8799 | 0.0235 | 0.0516 | 0.3552 | 0.0246 | 0.0545 | 0.7087 |
| IL-8/CXCL8 | 0.0039 | 0.0498 | 0.9875 | 0.0052 | 0.0096 | 0.3015 | 0.0038 | 0.0066 | 0.2537 |
| FAP | 0.0032 | 0.0024 | 0.0296 | 0.0032 | 0.0004 | 0.0009 | 0.0055 | 0.001 | 0.0888 |
| CCL2/JE/MCP-1 | 0.1875 | 0.1762 | 0.0237 | 0.2607 | 0.5408 | 0.1013 | 0.2381 | 0.4912 | 0.0148 |
| VEGF | 0.0057 | 0.0031 | 0.0747 | 0.0069 | 0.0006 | 0.0069 | 0.0124 | 0.0012 | 0.1608 |
| IL-17E/IL-25 | 0.5283 | 0.2059 | 0.3193 | 0.6005 | 0.291 | 0.1686 | 0.5841 | 0.2546 | 0.1798 |
| MMP-13 | 0.0025 | 0.0805 | 0.7462 | 0.0037 | 0.0203 | 0.2208 | 0.0023 | 0.0186 | 0.1334 |
| HGF | 0.2171 | 0.2133 | 0.1025 | 0.4735 | 0.1273 | 0.2466 | 0.5105 | 0.1599 | 0.3745 |
| CCL3/MIP-1 alpha | 0.1991 | 0.4181 | 0.0756 | 0.1838 | 0.3212 | 0.0188 | 0.0938 | 0.2491 | 0.0003 |
| FGF basic/FGF2/bFGF | 0.6789 | 0.2478 | 0.4913 | 0.8244 | 0.3643 | 0.3426 | 0.6923 | 0.2485 | 0.0533 |
| M-CSF | 0.0064 | 0.6624 | 0.5635 | 0.0055 | 0.3751 | 0.8558 | 0.0023 | 0.3239 | 0.4724 |
| CXCL9/MIG | 0.0053 | 0.0033 | 0.8661 | 0.0117 | 0.0006 | 0.335 | 0.029 | 0.0015 | 0.6411 |
| IL-12 p70 | 0.0279 | 0.0092 | 0.1643 | 0.0336 | 0.0162 | 0.0041 | 0.0336 | 0.0118 | 0.0758 |
| VCAM-1/CD106 | 0.3243 | 0.585 | 0.9488 | 0.1612 | 0.7387 | 0.2799 | 0.1365 | 0.7412 | 0.868 |
| ICAM-1/CD54 | 0.3625 | 0.0414 | 0.051 | 0.2063 | 0.2399 | 0.1661 | 0.1844 | 0.1981 | 0.0743 |
| TNFR1/TNFRSF1A | 0.0691 | 0.0152 | 0.051 | 0.1355 | 0.0072 | 0.0922 | 0.1293 | 0.0073 | 0.088 |
| TIMP-1 | 0.2638 | 0.1751 | 0.3618 | 0.3274 | 0.1435 | 0.0881 | 0.2707 | 0.1133 | 0.0381 |
| IGFBP-rp1/IGFBP-7 | 0.7765 | 0.6824 | 0.0483 | 0.5944 | 0.7384 | 0.2481 | 0.8589 | 0.9376 | 0.5456 |

TABLE 5
R² of linear regression for 20-plex analytes of microfluidic IFN-γ and 2D IFN-γ systems.

| T cell subset | | | | | | | | |
|---|---|---|---|---|---|---|---|---|
| CD3⁺ | | | CD4⁺ | | | CD8⁺ | | |
| hMSC:T cell ratio | | | | | | | | |
| 1:2 | 1:4 | 1:8 | 1:2 | 1:4 | 1:8 | 1:2 | 1:4 | 1:8 |

Microfluidic IFN-γ

| | | | | | | | | | |
|---|---|---|---|---|---|---|---|---|---|
| TNF-alpha | 0.2185 | 0.1018 | 0.006323 | 0.1945 | 0.1126 | 0.04014 | 0.1893 | 0.1145 | 0.02855 |
| IL-6 | 0.04585 | 0.000525 | 0.05859 | 0.04176 | 0.005558 | 0.007202 | 0.05532 | 0.01128 | 0.000304 |
| PD-L1/B7-H1 | 0.05703 | 0.01506 | 0.000899 | 0.06655 | 0.049 | 0.02606 | 0.06969 | 0.0493 | 0.005684 |
| IL-8/CXCL8 | 0.04135 | 0.02741 | 0.02001 | 0.03995 | 0.04411 | 0.02496 | 0.04621 | 0.04786 | 0.05856 |
| FAP | 0.05073 | 0.05409 | 0.06352 | 0.03657 | 0.05538 | 0.02918 | 0.05104 | 0.07124 | 0.2057 |
| CCL2/JE/MCP-1 | 0.1039 | 0.03836 | 4.68E−05 | 0.08347 | 0.0315 | 0.00433 | 0.1089 | 0.05124 | 0.07587 |
| VEGF | 0.01818 | 0.000633 | 0.002005 | 0.02751 | 0.0004 | 0.001076 | 0.0222 | 0.001226 | 0.02053 |
| IL-17E/IL-25 | 0.2765 | 0.1704 | 0.02528 | 0.2398 | 0.1773 | 0.06574 | 0.2463 | 0.1894 | 0.09045 |
| MMP-13 | 0.5664 | 0.3844 | 0.1581 | 0.4876 | 0.4747 | 0.2319 | 0.5326 | 0.4923 | 0.4596 |
| HGF | 0.005062 | 0.000686 | 0.08882 | 0.011 | 0.005616 | 0.03956 | 0.009839 | 0.00379 | 0.02243 |
| CCL3/MIP-1 alpha | 0.05858 | 0.001598 | 0.002331 | 0.0884 | 0.001314 | 0.008268 | 0.09112 | 0.000648 | 0.002388 |
| FGF basic/FGF2/bFGF | 0.000576 | 0.02658 | 9.74E−05 | 0.003575 | 0.02235 | 0.00823 | 0.005198 | 0.02375 | 0.00062 |
| M-CSF | 0.03064 | 0.02585 | 0.0148 | 0.02672 | 0.05188 | 0.0228 | 0.03847 | 0.06145 | 0.1065 |
| CXCL9/MIG | 0.008759 | 0.004578 | 4.25E−05 | 0.01702 | 0.001227 | 0.002026 | 0.01821 | 0.001898 | 0.000906 |
| IL-12 p70 | 0.02426 | 0.01596 | 0.001495 | 0.02477 | 0.02202 | 0.01406 | 0.02533 | 0.02627 | 0.001627 |
| VCAM-1/CD106 | 0.02698 | 0.008217 | 0.003744 | 0.02235 | 0.008862 | 0.000745 | 0.02129 | 0.009338 | 0.000513 |
| ICAM-1/CD54 | 0.09588 | 0.006992 | 0.001927 | 0.1197 | 0.04937 | 0.01927 | 0.1167 | 0.03923 | 0.000198 |
| TNFR1/TNFRSF1A | 0.1674 | 0.08774 | 0.007329 | 0.1541 | 0.1418 | 0.05763 | 0.1549 | 0.1354 | 0.04069 |
| TIMP-1 | 0.206 | 0.1872 | 0.07721 | 0.1697 | 0.1747 | 0.09996 | 0.1715 | 0.1802 | 0.1322 |
| IGFBP-rp1/IGFBP-7 | 0.0992 | 0.06174 | 0.01249 | 0.07559 | 0.06265 | 0.01734 | 0.07462 | 0.05666 | 0.02749 |

2D IFN-γ

| | | | | | | | | | |
|---|---|---|---|---|---|---|---|---|---|
| TNF-alpha | 0.00299 | 0.000114 | 0.05356 | 0.004907 | 0.002226 | 0.007615 | 0.00002453 | 0.00001473 | 0.2744 |
| IL-6 | 0.07094 | 5.97E−05 | 0.07558 | 0.05916 | 2.954E−08 | 0.04333 | 0.05689 | 0.00002059 | 0.04612 |
| PD-L1/B7-H1 | 0.1279 | 0.05418 | 0.000564 | 0.119 | 0.0893 | 0.02089 | 0.1173 | 0.08723 | 0.003439 |
| IL-8/CXCL8 | 0.1861 | 0.0906 | 6.01E−06 | 0.1753 | 0.1526 | 0.02601 | 0.1869 | 0.1663 | 0.03165 |
| FAP | 0.1934 | 0.2042 | 0.1103 | 0.1931 | 0.2681 | 0.2377 | 0.1734 | 0.2333 | 0.06898 |
| CCL2/JE/MCP-1 | 0.04198 | 0.04415 | 0.1186 | 0.03075 | 0.00919 | 0.06412 | 0.03378 | 0.01163 | 0.1364 |
| VEGF | 0.172 | 0.1937 | 0.07542 | 0.1649 | 0.2515 | 0.1649 | 0.1429 | 0.2271 | 0.0474 |
| IL-17E/IL-25 | 0.009768 | 0.03873 | 0.02419 | 0.006749 | 0.02716 | 0.04571 | 0.007369 | 0.03154 | 0.04344 |
| MMP-13 | 0.2016 | 0.07266 | 0.002583 | 0.1876 | 0.1245 | 0.03634 | 0.2048 | 0.1278 | 0.0541 |
| HGF | 0.03691 | 0.03752 | 0.06369 | 0.0126 | 0.05579 | 0.0326 | 0.01063 | 0.0476 | 0.01928 |
| CCL3/MIP-1 alpha | 0.0399 | 0.01606 | 0.075 | 0.04268 | 0.024 | 0.1274 | 0.06697 | 0.03226 | 0.2748 |
| FGF basic/FGF2/bFGF | 0.004223 | 0.03244 | 0.01162 | 0.001215 | 0.02011 | 0.02199 | 0.003859 | 0.03235 | 0.08809 |
| M-CSF | 0.1674 | 0.004695 | 0.008204 | 0.1729 | 0.01923 | 0.000815 | 0.2049 | 0.02374 | 0.01267 |
| CXCL9/MIG | 0.1745 | 0.1918 | 0.000702 | 0.1452 | 0.255 | 0.02269 | 0.1111 | 0.2209 | 0.005352 |
| IL-12 p70 | 0.1125 | 0.1544 | 0.04662 | 0.1054 | 0.1329 | 0.1843 | 0.1055 | 0.1447 | 0.07489 |
| VCAM-1/CD106 | 0.0237 | 0.007336 | 0.000102 | 0.04731 | 0.002743 | 0.02841 | 0.05327 | 0.00269 | 0.000681 |
| ICAM-1/CD54 | 0.02026 | 0.09755 | 0.08974 | 0.03866 | 0.03352 | 0.04624 | 0.04257 | 0.04007 | 0.07565 |
| TNFR1/TNFRSF1A | 0.07835 | 0.1353 | 0.0139 | 0.05352 | 0.1632 | 0.0676 | 0.05521 | 0.1627 | 0.06933 |
| TIMP-1 | 0.03035 | 0.04437 | 0.02033 | 0.0234 | 0.05146 | 0.0693 | 0.02951 | 0.06002 | 0.1007 |
| IGFBP-rp1/IGFBP-7 | 0.001988 | 0.004126 | 0.09177 | 0.006975 | 0.00275 | 0.0324 | 0.0007797 | 0.0001513 | 0.008976 |

### REFERENCES

[1] M. F. Pittenger, D. E. Discher, B. M. Péault, D. G. Phinney, J. M. Hare, A. I. Caplan, npj Regen. Med. 2019, 016960, DOI 10.1038/s41536-019-0083-6.
[2] O. Levy, R. Kuai, E. M. J. Siren, D. Bhere, Y. Milton, N. Nissar, M. De Biasio, M. Heinelt, B. Reeve, R. Abdi, M. Alturki, M. Fallatah, A. Almalik, A. H. Alhasan, K. Shah, J. M. Karp, Sci. Adv. 2020, 6, eaba6884.
[3] I. Martin, J. Galipeau, C. Kessler, K. Le Blanc, F. Dazzi, Sci. Transl. Med. 2019, 11, eaat2189.
[4] M. E. Wechsler, V. V. Rao, A. N. Borelli, K. S. Anseth, Adv. Healthc. Mater. 2021, 2001948, 1.
[5] J. Galipeau, Cytotherapy 2013, 15, 2.
[6] Food and Drug Administration (FDA), Oncologic Drugs Advisory Committee (ODAC). Meeting session on product characterization BLA 125706 applicant: Mesoblast, Inc, August, 2020. https://www.fda.gov/media/140988/download.
[7] C. A. Bravery, J. Carmen, T. Fong, W. Oprea, K. H. Hoogendoorn, J. Woda, S. R. Burger, J. A. Rowley, M. L. Bonyhadi, W. Van'T Hof, Cytotherapy 2013, 15, 9.
[8] Food and Drug Administration (FDA), Guidance for Industry: Potency Tests for Cellular and Gene Therapy Products. 2011, 27, 568-577.
[9] K. P. Robb, J. C. Fitzgerald, F. Barry, S. Viswanathan, Cytotherapy 2019, 21, 289.
[10] J. Galipeau, M. Krampera, J. Barrett, F. Dazzi, R. J. Deans, J. DeBruijn, M. Dominici, W. E. Fibbe, A. P. Gee, J. M. Gimble, P. Hematti, M. B. C. Koh, K. LeBlanc, I. Martin, I. K. McNiece, M. Mendicino, S. Oh, L. Ortiz, D. G. Phinney, V. Planat, Y. Shi, D. F. Stroncek, S. Viswanathan, D. J. Weiss, L. Sensebe, Cytotherapy 2016, 18, 151.
[11] R. Chinnadurai, D. Rajan, M. Qayed, D. Arafat, M. Garcia, Y. Liu, S. Kugathasan, L. J. Anderson, G. Gibson, J. Galipeau, Cell Rep. 2018, 22, 2455.
[12] R. Chinnadurai, I. B. Copland, S. R. Patel, J. Galipeau, J. Immunol. 2014, 192, 1491.
[13] D. C. Nguyen, S. Garimalla, H. Xiao, S. Kyu, I. Albizua, J. Galipeau, K. Y. Chiang, E. K. Waller, R. Wu, G. Gibson, J. Roberson, F. E. Lund, T. D. Randall, I. Sanz, F. E. H. Lee, Nat. Commun. 2018, 9, DOI 10.1038/s41467-018-05853-7.
[14] F. Nakahara, D. K. Borger, Q. Wei, S. Pinho, M. Maryanovich, A. H. Zahalka, M. Suzuki, C. D. Cruz, Z. Wang, C. Xu, P. E. Boulais, A. Ma'ayan, J. M. Greally, P. S. Frenette, Nat. Cell Biol. 2019, 21, 560.
[15] B. Trappmann, J. E. Gautrot, J. T. Connelly, D. G. T. Strange, Y. Li, M. L. Oyen, M. A. Cohen Stuart, H. Boehm, B. Li, V. Vogel, J. P. Spatz, F. M. Watt, W. T. S. Huck, Nat. Mater. 2012, 11, 642.
[16] J. Swift, I. L. Ivanovska, A. Buxboim, T. Harada, P. C. D. P. Dingal, J. Pinter, J. D. Pajerowski, K. R. Spinler, J. W. Shin, M. Tewari, F. Rehfeldt, D. W. Speicher, D. E. Discher, Science 2013, 341, DOI 10.1126/science.1240104.
[17] L. A. Low, D. A. Tagle, Lab Chip 2017, 17, 3026.
[18] J. Galipeau, L. Sensebe, Cell Stem Cell 2018, 22, 824.
[19] J. Q. Yin, J. Zhu, J. A. Ankrum, Nat. Biomed. Eng. 2019, 29.
[20] M. Najar, G. Raicevic, H. Fayyad-kazan, D. Bron, M. Toungouz, L. Lagneaux, Cytotherapy 2016, 18, 160.
[21] K. Le Blanc, L. Tammik, B. Sundberg, S. E. Haynesworth, O. Ringdén, Scand. J. Immunol. 2003, 57, 11.
[22] M. T. Pallotta, C. Orabona, C. Volpi, C. Vacca, M. L. Belladonna, R. Bianchi, G. Servillo, C. Brunacci, M. Calvitti, S. Bicciato, E. M. C. Mazza, L. Boon, F. Grassi, M. C. Fioretti, F. Fallarino, P. Puccetti, U. Grohmann, Nat. Immunol. 2011, 12, 870.
[23] S. Agaugué, L. Perrin-Cocon, F. Coutant, P. André, V. Lotteau, J. Immunol. 2006, 177, 2061.
[24] D. Braun, R. S. Longman, M. L. Albert, Blood 2005, 106, 2375.
[25] M. Frangois, R. Romieu-Mourez, M. Li, J. Galipeau, Mol. Ther. 2012, 20, 187.
[26] Q. Guan, Y. Li, T. Shpiruk, S. Bhagwat, D. A. Wall, Cytotherapy 2018, 20, 639.
[27] K. H. Vining, D. J. Mooney, Nat. Rev. Mol. Cell Biol. 2017, 18, 728.
[28] E. K. Williams, J. R. Garcia, R. G. Mannino, R. S. Schneider, W. A. Lam, A. J. Garcia, Integr. Biol. 2019, 11, 154.
[29] D. B. Chou, V. Frismantas, Y. Milton, R. David, P. Pop-Damkov, D. Ferguson, A. MacDonald, Ö. Vargel Bölükşι, C. E. Joyce, L. S. Moreira Teixeira, A. Rech, A. Jiang, E. Calamari, S. Jalili-Firoozinezhad, B. A. Furlong, L. R. O'Sullivan, C. F. Ng, Y. Choe, S. Marquez, K. C. Myers, O. K. Weinberg, R. P. Hasserjian, R. Novak, O. Levy, R. Prantil-Baun, C. D. Novina, A. Shimamura, L. Ewart, D. E. Ingber, Nat. Biomed. Eng. 2020, 4, 394.
[30] T. Tanzeglock, M. Soos, G. Stephanopoulos, M. Morbidelli, Biotechnol. Bioeng. 2009, 104, 360.
[31] C. J. Malemud, Biochem Pharmacol. 2019, 165, 33.
[32] M. Wang, E. R. Sampson, H. Jin, J. Li, Q. H. Ke, H. J. Im, D. Chen, Arthritis Res. Ther. 2013, 15, 1.
[33] Q. Hu, M. Ecker, Int. J. Mol. Sci. 2021, 22, 1.
[34] G. Kasper, J. D. Glaeser, S. Geissler, A. Ode, J. Tuischer, G. Matziolis, C. Perka, G. N. Duda, Stem Cells 2007, 25, 1985.
[35] C. Dezutter-Dambuyant, I. Durand, L. Alberti, N. Bendriss-Vermare, J. Valladeau-Guilemond, A. Duc, A. Magron, A. P. Morel, V. Sisirak, C. Rodriguez, D. Cox, D. Olive, C. Caux, Oncoimmunology 2016, 5, 1.
[36] A. Danilkovitch, Meet. Cell. Tissue Gene Ther. Advis. Committee; Feb. 9 2006.
[37] P. Kebriaei, L. Isola, E. Bahceci, K. Holland, S. Rowley, J. McGuirk, M. Devetten, J. Jansen, R. Herzig, M. Schuster, R. Monroy, J. Uberti, Biol. Blood Marrow Transplant. 2009, 15, 804.
[38] Mesoblast Inc., Remestemcel-L for treatment of steroid refractory acute graft versus host disease in pediatric patients. Meeting session with FDA Oncologic Drugs Advisory Committee (ODAC), August, 2020. https://www.fda.gov/media/140996/download.
[39] E. Dander, G. Lucchini, P. Vinci, M. Introna, F. Masciocchi, P. Perseghin, A. Balduzzi, S. Bonanomi, D. Longoni, G. Gaipa, D. Belotti, M. Parma, A. Algarotti, C. Capelli, J. Golay, A. Rovelli, A. Rambaldi, A. Biondi, E. Biagi, G. D'Amico, Leukemia 2012, 26, 1681.
[40] F. Yin, M. Battiwalla, S. Ito, X. Feng, F. Chinian, J. J. Melenhorst, E. Koklanaris, M. Sabatino, D. Stroncek, L. Samsel, J. Klotz, N. F. Hensel, P. G. Robey, A. J. Barrett, Stem Cells 2014, 32, 1278.
[41] Y. Liu, L. Wang, T. Kikuiri, K. Akiyama, C. Chen, X. Xu, R. Yang, W. J. Chen, S. Wang, S. Shi, Nat. Med. 2011, 17, 1594.
[42] A. L. Guangwen Ren, Xin Zhao, Liying Zhang, Jimin Zhang, W. Ling, J. Immunol. 2010, 184, 2321.
[43] F. Van Hauwermeiren, R. E. Vandenbroucke, C. Libert, Cytokine Growth Factor Rev. 2011, 22, 311.
[44] S. K. Williams, R. Fairless, O. Maier, P. C. Liermann, K. Pichi, R. Fischer, U. L. M. Eisel, R. Kontermann, A. Herrmann, B. Weksler, N. Romero, P. O. Couraud, K. Pfizenmaier, R. Diem, Sci. Rep. 2018, 8, 1.
[45] A. Martire, F. B. Bedada, S. Uchida, J. Poling, M. Kruger, H. Warnecke, M. Richter, T. Kubin, S. Herold, T. Braun, Basic Res. Cardiol. 2016, 111, DOI 10.1007/s00395-016-0573-2.
[46] S. Yang, J. Wang, D. D. Brand, S. G. Zheng, Front. Immunol. 2018, 9, DOI 10.3389/fimmu.2018.00784.
[47] A. J. Wang, A. Smith, Y. Li, J. F. Urban Jr, T. R. Ramalingam, T. A. Wynn, N. Lu, T. Shea-Donohue, Z. Yang, A. Zhao. Cell Biosci. 4, 1-10 (2014).
[48] Y. Maezawa, H. Nakajima, K. Suzuki, T. Tamachi, K. Ikeda, J. Inoue, Y. Saito, I. Iwamoto, J. Immunol. 176, 1013-1018 (2006).
[49] S. L. F. Pender, J. M. C. Fell, A. Ashkenazi, T. T. MacDonald, Gastroenterology 1998, 114, A1060.
[50] M. Fiedorczyk, P. A. Klimiuk, S. Sierakowski, E. Gindzienska-Sieskiewicz, J. Chwiecko, J. Rheumatol. 2006, 33, 1523.
[51] M. B. Murphy, K. Moncivais, A. I. Caplan, Exp. Mol. Med. 2013, 45, 1.
[52] F. D. C. Gonçalves, F. Luk, S. S. Korevaar, R. Bouzid, A. H. Paz, C. López-Iglesias, C. C. Baan, A. Merino, M. J. Hoogduijn, Sci. Rep. 2017, 7, 1.
[53] A. Galleu, Y. Riffo-vasquez, C. Trento, C. Lomas, L. Dolcetti, T. S. Cheung, M. Von Bonin, L. Barbieri, K. Halai, S. Ward, L. Weng, R. Chakraverty, G. Lombardi, F. M. Watt, K. Orchard, D. I. Marks, J. Apperley, M. Bornhauser, H. Walczak, C. Bennett, F. Dazzi, Sci. Transl. Med. 2017, 9, 1.
[54] S. F. H. de Witte, F. Luk, J. M. Sierra Parraga, M. Gargesha, A. Merino, S. S. Korevaar, A. S. Shankar, L. O'Flynn, S. J. Elliman, D. Roy, M. G. H. Betjes, P. N. Newsome, C. C. Baan, M. J. Hoogduijn, Stem Cells 2018, 36, 602.
[55] P. Pradhan, P. Chatterjee, H. Y. Stevens, C. Glen, C. Medrano-Trochez, A. Jimenez, L. Kippner, W. J. Seeto, Y. Li, G. Gibson, J. Kurtzberg, T. Kontanchek, C. Yeago, K. Roy, bioRxiv 2020.
[56] B. J. C. Quah, H. S. Warren, C. R. Parish, Nat. Protoc. 2007, 2, 2049.
[57] Pinho A G, Cibrão J R, Silva N A, Monteiro S, Salgado A J. Cell Secretome: Basic Insights and Therapeutic Opportunities for CNS Disorders. Pharmaceuticals (Basel). 2020; 13(2):31. Published 2020 Feb. 20. doi:10.3390/ph13020031

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A microfluidic potency assay comprising living cells, wherein the cells are encapsulated in a synthetic hydrogel, and further wherein the hydrogel is incorporated into a closed system which is perfused with liquid media and/or disease-relevant chemical stimuli.

2. The assay of claim 1, wherein the cells are differentiating cells.

3. The assay of claim 2, wherein the differentiating cells are human mesenchymal stromal cells.

4. The assay of claim 1, wherein the synthetic hydrogel comprises 4-arm maleimide-functionalized poly(ethylene-glycol) (PEG-4MAL).

5. The assay of claim 1, wherein the hydrogel is cross-linked with a protease-degradable peptide.

6. The assay of claim 5, wherein the peptide comprises VPM.

7. The assay of claim 6, wherein the peptide comprising VPM is GCRDVPMSMRGGDRCG (SEQ ID NO: 1).

8. The assay of claim 1, wherein the hydrogel is cross-linked with a dithiolated molecule.

9. The assay of claim 8, wherein the dithiolated molecule comprises dithiothreitol (DTT).

10. The assay of any one of claim 1, wherein the hydrogel comprises an adhesive peptide.

11. The assay of claim 10, wherein the adhesive peptide comprises RGD.

12. The assay of claim 11, wherein the adhesive peptide comprising RGD is GRGDSPC (SEQ ID NO: 2)

13. The assay of claim 1, wherein the liquid media comprises IFN-γ or other chemical stimuli relevant to disease indication.

14. The assay of claim 1, wherein the closed system comprised of silicone polydimethylsiloxane (PDMS).

15. A platform comprising the assay of claim 1.

16. The platform of claim 15, wherein the platform is high-throughput.

17. The platform of claim 15, wherein the platform is scalable.

18. A method of assessing viability, health, or in vivo secretion of a cell culture, the method comprising:
a. providing a microfluidic potency assay comprising living cells, wherein the cells are encapsulated in a polyethylene glycol (PEG) hydrogel, and further wherein the hydrogel is incorporated into a closed system which is perfused with liquid media and/or disease-relevant chemical stimuli;
b. obtaining one or more liquid samples from the cell culture; and
c. detecting one or more markers in the sample or samples obtained from the cell culture;
d. analyzing said markers to assess the viability, health, or in vivo secretion of the cell culture.

19. The method of claim 18, wherein the marker is an analyte produced by the cell.

20. The method of claim 19, wherein the analyte is part of the autocrine or paracrine system.

21-30. (canceled)
